SEQUENCE LISTING (36 sequences)

SEQ ID NO: 1 - DNA, 1692 bp, Mycobacterium B3683
atgactgaacaggactacagtgtctttgacgtagtggtggtagggagcggtgctgccggcatggtcgccgccctcaccgccgctcaccagggactctcgacagtagtcgttgagaaggctccgcactatggcggttccacggcgcgatccggcggcggcgtgtggattccgaacaacgaggttctgcagcgtgacggggtcaaggacacccccgccgaggcacgcaaatacctgcacgccatcatcggcgatgtggtgccggccgagaagatcgacacctacctggaccgcagtccggagatgttgtcgttcgtgctgaagaactcgccgctgaagctgtgctgggttcccggctactccgactactacccggagacgccgggcggtaaggccaccggccgctcggtcgagcccaagccgttcaatgccaagaagctcggtcccgacgagaagggcctcgaaccgccgtacggcaaggtgccgctgaacatggtggtgctgcaacaggactatgtccggctcaaccagctcaagcgtcacccgcgcggcgtgctgcgcagcatcaaggtgggtgtgcggtcggtgtgggccaacgccaccggcaagaacctggtcggtatgggccgggcgctgatcgcgccgctgcgcatcggcctgcagaaggccggggtgccggtgctgttgaacaccgcgctgaccgacctgtacctcgaggacggggtggtgcgcggaatctacgttcgcgaggccggcgcccccgagtctgccgagccgaagctgatccgagcccgcaagggcgtgatcctcggttccggtggcttcgagcacaaccaggagatgcgcaccaagtatcagcgccagcccatcaccaccgagtggaccgtcggcgcagtggccaacaccggtgacggcatcgtggcggccgaaaagctcggtgcggcattggagctcatggaggacgcgtggtggggaccgaccgtcccgctggtgggcgccccgtggttcgccctctccgagcggaactcccccgggtcgatcatcgtcaacatgaacggcaagcggttcatgaacgaatcgatgccctatgtggaggcctgccaccacatgtacggcggtcagtacggccaaggtgccgggcctggcgagaacgtcccggcatggatggtcttcgaccagcagtaccgtgatcgctatatcttcgcgggattgcagcccggacaacgcatcccgaagaaatggatggaatcgggcgtcatcgtcaaggccgacagcgtggccgagctcgccgagaagaccggtcttgcccccgacgcgctgacggccaccatcgaacggttcaacggtttcgcacgttccggcgtggacgaggacttccaccgtggcgagagcgcctacgaccgctactacggtgatccgaccaacaagccgaacccgaacctcggcgagatcaagaacggtccgttctacgccgcgaagatggtacccggcgacctgggcaccaagggtggcatccgcaccgacgtgcacggccgtgcgttgcgcgacgacaactcggtgatcgaaggcctctatgcggcaggcaatgtcagctcaccggtgatggggcacacctatcccggcccgggtggcacaatcggccccgccatgacgttcggctacctcgccgcgttgcatctcgctggaaaggcctga

SEQ ID NO: 2 - protein, 563 aa, Mycobacterium B3683
MTEQDYSVFDVVVVGSGAAGMVAALTAAHQGLSTVVVEKAPHYGGSTARSGGGVWIPNNEVLQRDGVKDTPAEARKYLHAIIGDVVPAEKIDTYLDRSPEMLSFVLKNSPLKLCWVPGYSDYYPETPGGKATGRSVEPKPFNAKKLGPDEKGLEPPYGKVPLNMVVLQQDYVRLNQLKRHPRGVLRSIKVGVRSVWANATGKNLVGMGRALIAPLRIGLQKAGVPVLLNTALTDLYLEDGVVRGIYVREAGAPESAEPKLIRARKGVILGSGGFEHNQEMRTKYQRQPITTEWTVGAVANTGDGIVAAEKLGAALELMEDAWWGPTVPLVGAPWFALSERNSPGSIIVNMNGKRFMNESMPYVEACHHMYGGQYGQGAGPGENVPAWMVFDQQYRDRYIFAGLQPGQRIPKKWMESGVIVKADSVAELAEKTGLAPDALTATIERFNGFARSGVDEDFHRGESAYDRYYGDPTNKPNPNLGEIKNGPFYAAKMVPGDLGTKGGIRTDVHGRALRDDNSVIEGLYAAGNVSSPVMGHTYPGPGGTIGPAMTFGYLAALHLAGKA

SEQ ID NO: 3 - protein, 563 aa, Mycobacterium avium paratuberculosis
MFYMSAQEYDVVVVGSGGAGMVAALTAAHRGLSTIVIEKAPHFGGSTARSGGGVWIPNNEVLKRDGVKDTPEAARTYLHGIIGDVVEPERIDTYLERGPEMLSFVLKHTPLKMCWVPRYSDYYPESPGGRAEGRSIEPKPFNARKLGPDEAGLEPAYGKVPLNVVVMQQDYVRLNQLKRHPRGVLRSLKVGARTMWAKATGKNLVGMGRALIGPLRIGLQRAGVPVVLNTALTDLYLEDGVVRGVYVRDSQAAESAEPRLIRARRGVILASGGFEHNEQMRVKYQRAPITTEWTVGAKANTGDGILAAEKLGAALELMEDAWWGPTVPLVGAPWFALSERNSPGSIIVNMSGKRFMNESMPYVEACHHMYGGEFGQGPGPGENIPAWLVFDQQYRDRYIFAGLQPGQRIPRKWLESGVIIQADTLEELASRAGLPVDEFLATVQRFNGFARTGIDEDYHRGESAYDRYYGDPTNKPNPNLGEISHPPYYAAKMVPGDLGTKGGIRTDIHGRALRDDGSIIEGLYAAGNVSAPVMGHTYPGPGGTIGPAMTFGYLAALHIAGEN

SEQ ID NO: 4 - protein, 566 aa, Mycobacterium tuberculosis
MFYMTVQEFDVVVVGSGAAGMVAALVAAHRGLSTVVVEKAPHYGGSTARSGGGVWIPNNEVLKRRGVRDTPEAARTYLHGIVGEIVEPERIDAYLDRGPEMLSFVLKHTPLKMCWVPGYSDYYPEAPGGRPGGRSIEPKPFNARKLGADMAGLEPAYGKVPLNVVVMQQDYVRLNQLKRHPRGVLRSMKVGARTMWAKATGKNLVGMGRALIGPLRIGLQRAGVPVELNTAFTDLFVENGVVSGVYVRDSHEAESAEPQLIRARRGVILACGGFEHNEQMRIKYQRAPITTEWTVGASANTGDGILAAEKLGAALDLMDDAWWGPTVPLVGKPWFALSERNSPGSIIVNMSGKRFMNESMPYVEACHHMYGGEHGQGPGPGENIPAWLVFDQRYRDRYIFAGLQPGQRIPSRWLDSGVIVQADTLAELAGKAGLPADELTATVQRFNAFARSGVDEDYHRGESAYDRYYGDPSNKPNPNLGEVGHPPYYGAKMVPGDLGTKGGIRTDVNGRALRDDGSIIDGLYAAGNVSAPVMGHTYPGPGGTIGPAMTFGYLAALHIADQAGKR

SEQ ID NO: 5 - protein, 571 aa, Nocardia farcinica
MTDPVLDPHSYDVVVVGSGAAGMTAALTAAHHGLRVVVLEKAAHYGGSTARSGGGVWIPGNKALRASGRPDDREEARTYLHSIIGDVVPKERIDTYIDRGAEAFDFVLDHTPLQMKWVPGYSDYYPEAPGGRGEGRSCEPKPFDLKVLGPEKDKLEPAYAKAPLNVVVMQADFVRLNLIRRHPKGMLRAMRVGARTYWAKFTGKHIVGMGQAIIAAMRKGLMDANVPLLLNTPMTKLVVEDGRVTGVEALHEGEPVVFSARYGVVLGSGGFEHNAEMRAKYQRQPITTEWTTGAAANTGDGIRAGMEIGADVDFMEDAWWGPTIFKGGRPWFALAERNLPGCVIVNAQGKRFANESAPYVEAVHAMYGGEYGQGEGPGENIPAWLVFDQRYRNRYIFAGLQPGQRFPSRWMEDQNIVKADTLAELAELIGVPVGNLTATVERFNKFAETGKDEDFGRGDSHYDRYYGDPTVKPNPCLAALVQGPFYAAKIVPGDLGTKGGLVADESGRVLREDGSPIPGLYASGNCSTPVMGHTYAGPGATIGPAITFGYLSVLDILARKNEQSPAASGTA

SEQ ID NO: 6 - protein, 535 aa, Streptomyces avermitilis
MTAALTAAKQGLSCVVVEKAATFGGSAARSGAGIWIPNNPVILAAGVPDTPAKAAAYLAAVVGPDVSADRQRAFLGHGPAMISFVMANSPLRFRWMEGYSDYYPELSGGLPNGRSIEPDQLDGNILGAELAHLNPSYMAVPAGMVVFSADYKWLTLSAVSAKGLAVAAECLARGTKAALLGQKPLTMGQSLAAGLRAGLLAAQVPVWLNTPLTDLYRENGTVTGAVVAKGGSAGLVRARHGVVVGSGGFEHNAAMRDQYQRQPIGTAWTVGAKENTGDGIRAGERAGAALDLMDDAWWGPTIPLPDQPYFCLAERTLPGGLLVNAAGARFVNEAAPYSDVVHTMYERNPTAPDIPAWLIVDQNYRNRYLFKDVAPTLAFPGSWYDSGAAHKAWTLDALAGRIGMPAAALRATVNRFNSLALSGDDTDFQRGDSTYDHYYTDPAIVPNSCLAPLWLAPYYAFKIVPGDLGTKGGLRTDARARVLRADGSVIPGLYAAGNASAAVMGHSYAGAGSTIGPAMTFGYIAALDIAAAAGS

SEQ ID NO: 7 - protein, 565 aa, Rhodococcus erythropolis
MAKNQAPPATQAKDIVVDLLVIGSGTGMAAALTANELGLSTLIVEKTQYVGGSTARSGGAFWMPANPILAKAGAGDTVERAKTYVRSVVGDTAPAQRGEAFVDNGAATVDMLYRTTPMKFFWAKEYSDYHPELPGGSAAGRTCECLPFDASVLGAERGRLRPGLMEAGLPMPVTGADYKWMNLMVKKPSKAFPRIIRRLAQGVYGKYVLKREYIAGGQALAAGLFAGVVQAGIPVWTETSLVRLITEDGRVTGAVVVQDGREVTVTARRGVVLAAGGFDHNMEWRHKYQSESLGEHESLGAEGNTGEAIEAAQELGAGIGSMDQSWWFPAVASIKGRPPMVMLAERALPGSFIVDQTGRRFVNEATDYMSFGQRVLEREKAGDPAESMWFVFDQEYRNSYVFAGGIFPRQPLPQAFFESGIAHQASSPAELARKVGLPEDAFAESFQKFNEAAAAGSDAEFGRGGSAYDRYYGDPTVSPNPNLRQLDKSALYAVKMTLSDLGTCGGVQADENARVLREDGSVIDGLYAIGNTAANAFGHTYPGAGATIGQGLVYGYIAAHHAAEK

SEQ ID NO: 8 - protein, 576 aa, Comomonas testosteroni ksdA
MAEQEYDLIVVGSGAGAMLGAIRAQEQGLKTLVVEKTELFGGTSALSGGGIWIPLNYDQKTAGIKDDLETAFGYMKRCVRGMATDDRVLAYVETASKMAEYLRQIGIPYRAMAKYADYYPHIEGSRPGGRTMDPVDFNAARLGLAALETMRPGPPGNQLFGRMSISAFEAHSMLSRELKSRFTILGIMLKYFLDYPWRNKTRRDRRMTGGQALVAGLLTAANKVGVEMWHNSPLKELVQDASGRVTGVIVERNGQRQQINARRGVLLGAGGFERNQEMRDQYLNKPSKAEWTATPVGGNTGDAHRAGQAVGAQLALMDWSWGVPTMDVPKEPAFRGIFVERSLPGCMVVNSRGQRFLNESGPYPEFQQAMLAEHAKGNGGVPAWIVFDASFRAQNPMGPLMPGSAVPDSKVRKSWLNNVYWKGETLEDLARQIGVDATGLQDSARRMTEYARAGKDLDFDRGGNVFDRYYGDPRLKNPNLGPIEKGPFYAMRLWPGEIGTKGGLLTDREGRVLDTQGRIIEGLYCVGNNSASVMGPAYAGAGSTLGPAMTFAFRAVADMLGKPLPIENPHLLGKTV

SEQ ID NO: 9 - DNA, 1203 bp, Mycobacterium B3683
ttgggtttgcgtggtgacgcagcgatcgtcgggtttcacgagctacctgcgacgcggaagccgaccgggaccgcggagttcaccatcgaacagtgggcgcggttggcggccgcggcggtggccgacgcggggctgtcggtccagcaggtcgacgggctggtgacctgcggggtcatggagtcccagctgttcgtcccctccacagtcgccgagtatctgggtctggcggtcaatttcgccgagatcgtcgatctcggcggcgcctcgggcgcggccatggtgtggcgcgcggcggcggcgatcgaactggggctctgccaggcggtgctgtgcgccatcccagccaactacctgaccccgatgtcggcggagcgtccctacgatcccggcgacgcgctgtactacggggcgtccagcttccggtacggctcgccgcaggccgagttcgagattccctacggctacctcggacagaacggtccgtacgcgcaggtcgcccagatgtactcggccgcatacggatacgacgagaccgcgatggccaagatcgtcgtcgaccagcgggtgaacgccaaccacacacccggggcggtgttccgggacaaaccggtgaccatcgccgatgtcctggacagcccgatcatcgcgtctccgctgcacatgctggaaatcgtcatgccgtgcatggggggatcggcagtgctcgtcaccaatgccgaactggcccgcgccggccgccaccgaccggtctggatcaaggggttcggcgaacgggtgccctacaagtccccggtctatgccgccgatccgctccagacaccgatggtgaaggtcgccgaatccgccttcgggatggccggcctgaccccggccgacatggacatggtgtcgatctacgactgctacaccatcaccgccctgctgacgttggaggacgcgggtttctgtgccaagggcacgggaatgcggttcgtcaccgaccacgacctgaccttccgcggtgacttcccgatgaacaccgcaggcggacagctcggctacggccagcccggcaatgccggtggcatgcaccatgtgtgcgatgccacccggcagctgatgggacgcgccggggcaacccaggtcgcggactgtcaccgcgccttcgtctcgggcaacggtggcgtgctcagcgaacaagaagctctcgtcctggagggggat

SEQ ID NO: 10 - protein, 401 aa, Mycobacterium B3683
MGLRGDAAIVGFHELPATRKPTGTAEFTIEQWARLAAAAVADAGLSVQQVDGLVTCGVMESQLFVPSTVAEYLGLAVNFAEIVDLGGASGAAMVWRAAAAIELGLCQAVLCAIPANYLTPMSAERPYDPGDALYYGASSFRYGSPQAEFEIPYGYLGQNGPYAQVAQMYSAAYGYDETAMAKIVVDQRVNANHTPGAVFRDKPVTIADVLDSPIIASPLHMLEIVMPCMGGSAVLVTNAELARAGRHRPVWIKGFGERVPYKSPVYAADPLQTPMVKVAESAFGMAGLTPADMDMVSIYDCYTITALLTLEDAGFCAKGTGMRFVTDHDLTFRGDFPMNTAGGQLGYGQPGNAGGMHHVCDATRQLMGRAGATQVADCHRAFVSGNGGVLSEQEALVLEGD

SEQ ID NO: 11 - protein, 401 aa, Mycobacterium B3683
LGLRGDAAIVGFHELPATRKPTGTAEFTIEQWARLAAAAVADAGLSVQQVDGLVTCGVMESQLFVPSTVAEYLGLAVNFAEIVDLGGASGAAMVWRAAAAIELGLCQAVLCAIPANYLTPMSAERPYDPGDALYYGASSFRYGSPQAEFEIPYGYLGQNGPYAQVAQMYSAAYGYDETAMAKIVVDQRVNANHTPGAVFRDKPVTIADVLDSPIIASPLHMLEIVMPCMGGSAVLVTNAELARAGRHRPVWIKGFGERVPYKSPVYAADPLQTPMVKVAESAFGMAGLTPADMDMVSIYDCYTITALLTLEDAGFCAKGTGMRFVTDHDLTFRGDFPMNTAGGQLGYGQPGNAGGMHHVCDATRQLMGRAGATQVADCHRAFVSGNGGVLSEQEALVLEGD

SEQ ID NO: 12 - protein, 402 aa, Mycobacterium avium paratuberculosis
MGLRGEAAIVGYVELPPERLSKASPAPFVLEQWAEPGAAALQDAGLPGEVVNGIVASHLAESEIFVPSTIAEYLGVGARFAEHVVLGGASAAAMVWRAAAAIELGICDAVLCALPARYITPSSKKKPRPMVDAMFFGSSSNQYGSPQAEFEIPYGNLGQNGPYGQVAQRYAAVYGYDERAMAKIVVDQRVNANHTDGAIWRDTPLTVEDVLASPVIADPLHMLEIVMPCVGGAAVVVANADLAKRARHRPVWVKGFGEHVPFKTPTYAEDLLRTPIAAAADTAFAMTGLSRAQMDMVSIYDCYTITVLLSLEDAGFCEKGRGMEFVADHDLTFRGDFPLNTAGGQLGFGQAGLAGGMHHVCDATRQIMGRAGAAQVPDCNRAFVSGNGGILSEQTTLILEGD

SEQ ID NO: 13 - protein, 400 aa, Mycobacterium avium paratuberculosis
MTGLRGEAAIVGIAELPAERRPTGPPRFTLDQYALLAKLVIEDAGVDPGRVNGLLTHGVAESAMFAPATLCEYLGLACDFGERVDLGGASSAGMVWRAAAAVELGICEAALAVVPGSASVPHSARRPPPESNWYGASSNNYGSPQAEFEIPYGNVGQNAPYAQIAQRYAAEFGYDPAALAKIAVDQRTNACAHPGAVFFGTPITAADVLDSPMIADPIHMLETVMRVHGGAAVLIANADLARRGRHRPVWIKGFGEHIAFKTPTYAEDLLSTPIARAAERAFAMAGLDRPDVDVASIYDCYTITVLMSLEDAGFCAKGQGMQWIGDHDLTHRGDFPLNTAGGQLSFGQAGMAGGMHHVVDGARQIMGRAGDAQVPGCHTAFVTGNGGIMSEQVALLLQGE

SEQ ID NO: 14 - protein, 383 aa, Polaromonas sp.
MIVGVADLPLKDGKVLRPMSVLEAQALVARDALKDAGIPMSEVDGLLTAGLWGVPGPGQLPTVTLSEYLGITPRFIDSTNIGGSAFEAHVAHAAMAIEAGRCEVALITYGSLQKSEMSRNLAGRPAVLTMQYETPWGMPTPVGGYAMAAKRHMHEYGTTSEQLAEIAVATRQWAALNPAATMRDPLSIEDVLKSPMVCDPMHLLDICLVTDGGGAVVMTTAEHARALGRKAVHVRGYGESHTHWTIAAMPDLARLTAAEVAGRDAFAMAGIGHDAIDVVEVYDSFTITVLLTLEALGFCQRGESGAFVSNQRTAPGGAFPLNTNGGGLSYAHPGMYGIFLLIEAVRQLRGECGPRQIANAVTALVHGTGGTLSSGATCILSTR

SEQ ID NO: 15 - protein, 387 aa, Ralstonia eutropha
MTLNGSAYIVGAYEHPTRKADDLSVARLHADVARGALADAGLTAADVDGYFCAGDAPGLGTTTIVEYLGLKPRHVDSTECGGSAPILHVAHAAEAIAAGRCNVALITLAGRPRAAGAALALRAPDPDAPDVAFELPFGPATQNLYGMVAKRHMYEFGTTSEQLAWIKVAASHHAQHNPHAMLRNVVTVEDVVNSPMVADPLHRLDCCVMSDGGGALIVARPEIARQLRRPLVKVRGTGEAPKHAMGGNIDLTWSAAAWSGPAAFAEAGVTPADIKYASLYDSFTITVLMQLEDLGFCKKGEGGKFVADGGLISGVGRLPFNTDGGGLCNNHPANRGGVTKVIEAVRQLRGEAHPAVQVSNCDLALASGIGGALASRHTAATLILERE

SEQ ID NO: 16 - protein, 404 aa, Rhodopseudomonas palustris
MDSGLAPRGAPRNDERDGVCNRQAAIMSYITGVGLTRFGKIDGSTTLSLMREAAEAAIADAGLKRGDIDGLLCGYSTTMPHIMLATVFAEHFGILPSHCHAVQVGGATGMAMAMLAYQLVESGAAKNILVVGGENRLTGQSRDASVQALAQVGHPIYEVPLGPTIPAYYGLVASRYMHDHGVTEEDLAEFAVLMRSHAITHPGAQFHEPISVAEVMASKPIASPLKLLDCCPVSDGGAALVISREPTTAHQIKVRGCGQAHTHQHVTAMPAAGPSGAELSIARAWATSGVEIADVKYAAVYDSFTITLLMLLEDLGLAARGEAAARARDGYFSRTGAMPLNTHGGLLSYGHCGVGGAMAHLVETHLQMTGRAGDRQVRDASLALLHGDGGVLSSHVSMILERVR

SEQ ID NO: 17 - DNA, 414 bp, Mycobacterium B3683
atgaccgagtcgtcggcccggccagtgccactgcccacgccgacctcggcaccgttctgggatggcctgcgccggcacgaggtgtgggtgcaattctcaccgtcatcggatgcctacgtgttctatccgcgcatcctggcgcccggcaccctggccgatgatctgtcctggcgccagatctccggtgatgccaccctggtcagcttcgccgtcgcacagcgaccggtcgcccctcagttcgccgatgccgttccgcatctgctcggcgtggtgcagtggaccgaggggccgcggctggccaccgagatcgtcggcgtcgatccggctcgactgcgcatcggtatggccatgacgccggtgttcaccgaacccgacggcgccgatatcaccctgttgcactacaccgccgccgaa

SEQ ID NO: 18 - protein, 137 aa, mycobacterium B3683
MTESSARPVPLPTPTSAPFWDGLRRHEVWVQFSPSSDAYVFYPRILAPGTLADDLSWRQISGDATLVSFAVAQRPVAPQFADAVPHLLGVVQWTEGPRLATEIVGVDPARLRIGMAMTPVFTEPDGADITLLHYTAA

SEQ ID NO: 19 - protein, 137 aa, Mycobacterium avium paratuberculosis
MTTFERPMPVKTPTTAPFWDALAQHRIVIQYSPSLQSYVFYPRVRAPRTLADDLEWREISGMGSLYSYTVAHRPVSPHFADAVPQLLAIVEWDEGPRFSTEMVNVDPAQLRVGMRVQPVFCDYPEHDVTLLRYQPAD

SEQ ID NO: 20 - protein, 138 aa, Ralstonia eutropha
MAIGHYMDTAAFWAATRERRLLVQFCTQTGRWQAYPRPGSVYTGRRRLAWREVSGDGVLASWTVDRMNTPAAADAPRMHAWIDLVEGARILSWLVDCDPARLRVGLAVRVAWISLPDGWQWPAFTIAAHSGGPNGKAP

SEQ ID NO: 21 - protein, 133 aa, Polaromonas sp.
MYDKPLPVIDGESRPYWDALKQHRLTLKRCQDCGKHHFYPRALCPHCHSDAVEWVDACGTGTIYSYTIARRPAGPAFKADTPYVVAVIDLDEGARMMTNIVTDDVEAVRIGQRVTVQYDDVTEEVTLPKFRLL

SEQ ID NO: 22 - protein, 135 aa, Streptomyces avermitilis
MSGRRFDEPETDAFTRPYWDAAAEGVLLLRRCAGCGRTHHYPREFCPHCWSDDVTWERASGRATLYTWSVVHRNDLPPFGERTPYVAAVVDLAEGPRMMTEVVECAAAELRVGMELEAAFRPAGEVTVPVFRPRG

SEQ ID NO: 23 - protein, 143 aa, Mycobacterium avium paratuberculosis
HCRQCLSDDIGWQQSGGRGEIYSWTVVHRPVTAEFIPPNAPAIITLDEMTAEPLRPQTGPVPHASSPLSVPFWEGCRSRQLRYQRCRACDLANFPPTEGYQMLTNVVGVPPGDLRVGLRVRVQFHTVAADVTLPYFTDETDGS

SEQ ID NO: 24 - DNA, 2211 bp, Mycobacterium B3683
atggcgctggcactcaccgatgaacaggtacagctgaccgaggcgatggcgggtttcgcccgcaggcacggcggactggaactgacccggtcgcagttcgacgccctcgcagccggggaacgcccggcgttctgggcggccttggtcgccaacggactgcacggggttcaattgcccgagcagggtgggggtttcgtcgatgccgcctgcgtcatcgacgccgcgggctacggtctgctgcccggcccgctgctgcccacgatgatcgccggtgccgtcattgcagacctgccggaacaaccggcggtgcgcgccgcgcgcgaggccctcgccgcgggtggcccgatggcggtgttgctgccgagcgatggcgtgctgcgggccgaacccgacggcgcagggtggcggctgaccggcgcggccggaccgcagctcggcgtggccgccgcggagcatgtgatcgttgccgccgataccgatgcggcgcaaagactctggtttctgatcaacgctgccgggccgggggtggtggtgcaggcggccgccccgaccgatctgacccgggatgtcggcaccctgtcgtgcgccgacgcacccgtcgcggccgatgccgtgctggccggtgtcgacccggtgcgggcgcggtgccatgcgatcggcctgatggcggccgaggcagcggggatcgcgcgctggtgtgtggacaatgtggtcgcctatctgaaggtgcgcgaacagttcggacgccgcatcggggcgttccaggccctgcagcacaaggcggccatgctgttcatcgacagtgaacttgccgccgccgccgcatgggatgcggtgcgcggcgccgaacaaccgatcgagcaacacgagatcgccgccgcaggcgctgccatcgcggcgatcggcaagctgccggatctggtggtcgatgcgctgacgatgttcggggccatcgggtacacctgggagcacgacctgcacctgtactggaagcggtcgatcagcctggccgccgccgcgggcggtgtcgccgaatgggccgagctgctcggggaacccgaccggcagccaagagatttcggcatcgagctggccggtgtggaagagcggttccgggggcagatcgccgcgctgatcgacgccgcggcgcagctggacaacgaggcgccgggccggcagaaccccgagtacgaggacttctggaccggtccgcgccggaccgcactggccgatgccggactcgtcgcgccatatctgcccgcgccgtgggggctggacgccacgccggcccaacagctcgtcatcgacgaggaattcgaccggcggccaacgcttacccggccatcgttgggaatcgcacagtggatactgccgacggttatcgccgaaggcaccgacggccaacgggagcgcttcgcggtgccgacgctgcgcggtgagatcgggtggtgtcagctgttctccgaacccggcgccggatcggatctggcgtccttgacgaccagggcgaccaaggtcgagggcggctggcggatcgacgggcagaaggtgtggacctcctcggcgcagcgcgccgactggggtgcgctgctggccaggacggatccgcaggccgccaagcaccggggcatcggctacttcctgatcgatatgacgagcccgggcatcaccatccggccgctgcgaaccgccagcggtgacgagcatttcaacgaggtgttcttcgacgatgtcttcgtgcccgatgacatgctggtcggtgagccgaccgcgggctggtcgcatgcgctggccacgatggccaacgaacgggtggccatcggtgcctacgccaaactggacaaggaacgtgaattgcgggcgctggcccgtcaggccggtccggcgggtgtcatggtgcggcacgcgttgggccgggtacgggccgccaccaacgccatcggcgcgctcgcggtgcgcgacaccctgcgccggctcgccggacacgggcccggcccggcgtccagcgtcggcaaggtcggcaccgcactgttggtgcgccgggtgaccgccgacgcgctggctttcagcggtcgggccgccatggtgggtggcgccgaccaccccgcagtggccgacacgttgatgatgcctgcggaggtcatcggcggtggcaccgtcgagatccagctcaatatcatcgccaccatgatcctcggactaccgcgcgca

SEQ ID NO: 25 - protein, 737 aa, Mycobacterium B3683
MALALTDEQVQLTEAMAGFARRHGGLELTRSQFDALAAGERPAFWAALVANGLHGVQLPEQGGGFVDAACVIDAAGYGLLPGPLLPTMIAGAVIADLPEQPAVRAAREALAAGGPMAVLLPSDGVLRAEPDGAGWRLTGAAGPQLGVAAAEHVIVAADTDAAQRLWFLINAAGPGVVVQAAAPTDLTRDVGTLSCADAPVAADAVLAGVDPVRARCHAIGLMAAEAAGIARWCVDNVVAYLKVREQFGRRIGAFQALQHKAAMLFIDSELAAAAAWDAVRGAEQPIEQHEIAAAGAAIAAIGKLPDLVVDALTMFGAIGYTWEHDLHLYWKRSISLAAAAGGVAEWAELLGEPDRQPRDFGIELAGVEERFRGQIAALIDAAAQLDNEAPGRQNPEYEDFWTGPRRTALADAGLVAPYLPAPWGLDATPAQQLVIDEEFDRRPTLTRPSLGIAQWILPTVIAEGTDGQRERFAVPTLRGEIGWCQLFSEPGAGSDLASLTTRATKVEGGWRIDGQKVWTSSAQRADWGALLARTDPQAAKHRGIGYFLIDMTSPGITIRPLRTASGDEHFNEVFFDDVFVPDDMLVGEPTAGWSHALATMANERVAIGAYAKLDKERELRALARQAGPAGVMVRHALGRVRAATNAIGALAVRDTLRRLAGHGPGPASSVGKVGTALLVRRVTADALAFSGRAAMVGGADHPAVADTLMMPAEVIGGGTVEIQLNIIATMILGLPRA

SEQ ID NO: 26 - protein, 678 aa, Mycobacterium avium paratuberculosis MAP4303C
MTLGLSPEQQELGDAVGQFAARNAPIAATRDSFAELAAGRLPRWWDGLVANGFHAVHLPEELGGQGGRLMDAACVLESAGKSLLPGPLLPTVAAGAVALLADPAPAARSVLRDLAAGIPAAVVLPGDGDLHAGAGDGHWLLSGASEVTAGVCAARIVLVGARTRDGELVWAAVDTEKPTATVEPISGTDLVADAGVLRLDNHRVLDSEVLTGIDPERARCVVLGLVAATTAGVIQWCVQAVTAHLRIREQFGKVIGTFQALQHSAAMLLVSSELATAAAWDAVRAGDESLEQHRMAAAGAAVMAISPAPDLVLDALTMFGAIGFTWEHDLHLYWRRAISLAASIGPANRWARRLGELTCTRQRDMAVNLGDAESELRAKVAETLDAALELRNDQPGRQGDYSEFETGPQRTLISDAGLIAPHWPKPWGLDAGPLRQLIIDDEFAKRPALVRPSLGIAEWILPSVIRAAPKDLQEKLIPPTLRGEIAWCQLFSEPGAGSDLAALSTRATKVDGGWTINGHKIWTSAAHRADYGALLARTDPQAGKHRGIGYFVVDMRSAGIEVQPIKTATGDAHFNEVFLTDVFVPDDMLLGEPTGGWNLAIATMAEERSAISGYVKFDRAAALRRLAAQPGPDRDDALRELGRLDAYTTRSRRWECARPSGCSTARRPGRRPASPRWR

SEQ ID NO: 27 - protein, 734 aa, Nocardia farcinica
MIVPVALTADQAALAESVGGFAARHATREYTRRNTEQLKRGERPAFWPELVATGLTGVHLPDEVGGQGGAVADIAVVVAEAGRALLPGPLLPSVVASAIVATAATGAGTEKALRHFAEGGTGAVLLPEHGVAVSGGEARLSGRSGLVLGAPGAELFVVAAGSRWFLVERSAPGVGVEIEDGADLGRDLGRVAFQDVTPAAELDGIDGDRAADIAVAFLAVEAAGVIRWCSDTATEYVQARKQFGRPIGAFQAVQHRTAQLLITSELATAAAWDAVRGLDDEPDQRAHAVAGAALITLGNAVHAAVECLALHGAIGFTWEHDLHLYWRRAITLAGLAGPGERWERRLGEVALRGPRTFTVPLPETDTTFRQWVSGILDTAAELTNPHPSTIGDHDSVNTGPRRTLLADHGLVSPPMPRPYGIEAGPLEQLILQDEYDRHGIAQPSMGIGQWVVPIVLQRGTPAQLERLAGPALRGEEIWCQLFSEPEAGSDVASLSLRATKVDGGWQLNGQKIWTTLAHRSDWGLLLARTDPEAERHRGLTMFLVDMHAPGVDVRPITQSSGDAEFNEVFFDDAFVPDDMVLGEPGQGWALTLETLAQERLFIGGVRDPGHNQRIREIIEREEYAGSRDEALRTLGRISARGAAISAMNLRETIRRLDGQGVGPGTSIAKAAAAMLHTDAAAAALELIGPAAALSEARSEVVHHELDIPTWVIGGGTLEIQLNTIATLVMGLPRK

SEQ ID NO: 28 - protein, 711 aa, Mycobacterium tuberculosis
MVATVTDEQSAARELVRGWARTAASGAAATAAVRDMEYGFEEGNADAWRPVFAGLAGLGLFGVAVPEDCGGAGGSIEDLCAMVDEAARALVPGPVATTAVATLVVSDPKLRSALASGERFAGVAIDGGVQVDPKTSTASGTVGRVLGGAPGGVVLLPADGNWLLVDTACDEVVVEPLRATDFSLPLARMVLTSAPVTVLEVSGERVEDLAATVLAAEAAGVARWTLDTAVAYAKVREQFGKPIGSFQAVKHLCAQMLCRAEQADVAAADAARAAADSDGTQLSIAAAVAASIGIDAAKANAKDCIQVLGGIGCTWEHDAHLYLRRAHGIGGFLGGSGRWLRRVTALTQAGVRRRLGVDLAEVAGLRPEIAAAVAEVAALPEEKRQVALADTGLLAPHWPAPYGRGASPAEQLLIDQELAAAKVERPDLVIGWWAAPTILEHGTPEQIERFVPATMRGEFLWCQLFSEPGAGSDLASLRTKAVRADGGWLLTGQKVWTSAAHKARWGVCLARTDPDAPKHKGITYFLVDMTTPGIEIRPLREITGDSLFNEVFLDNVFVPDEMVVGAVNDGWRLARTTLANERVAMATGTALGNPMEELLKVLGDMELDVAQQDRLGRLILLAQAGALLDRRIAELAVGGQDPGAQSSVRKLIGVRYRQALAEYLMEVSDGGGLVENRAVYDFLNTRCLTIAGGTEQILLTVAAERLLGLPR

SEQ ID NO: 29 - protein, 731 aa, Mycobacterium tuberculosis
MSIAITPEHYELADSVRSLVARVAPSEVLHAALESPVENPPPYWQAAAEQGLQGVHLAESVGGQGFGILELAVVLAEFGYGAVPGPFVPSAIASALIAAHDPQAKVLAELATGAAIAAYALDSGLTATRHGDVLVIRGEVRAVPAAAQASVLVLPVAIESRDEWVVLRNDQLEIEAVKSLDPLRPIAHVRANAVDVSDDALLSNLTMTTAHALMSTLLSAEAVGVARWATDTASAYAKIREQFGRPIGQFQAIKHKCAEMIADTERATAAVWDAARALDDAGESSSDVEFAAAVAATLAPATAQRCTQDCIQVHGGIGFTWEHDTNVYYRRALMLAACFGRGSEYPQRVVDTATTAGMRPVDIDLDPSTEKLRAQIRAEVAALKAMPREPRTVAIAEGGWVLPYLPKPWGRAASPVEQIIIAQEFTAGRVKRPQIAIATWIVPSIVAFGTDNQKQRLLPPTFRGDIFWCQLFSEPGAGSDLASLATKATRVDGGWRITGQKIWTTGAQYSQWGALLARTDPSAPKHNGITYFLLDMKSEGVQVKPLRELTGKEFFNTVYLDDVFVPDELVLGEVNRGWEVSRNTLTAERVSIGGSDSTFLPTLGEFVDFVRDYRFEGQFDQVARHRAGQLIAEGHATKLLNLRSTLLTLAGGDPMAPAAISKLLSMRTGQGYAEFAVSSFGTDAVIGDTERLPGKWGEYLLASRATTIYGGTSEVQLNIIAERLLGLPRDP

SEQ ID NO: 30 - protein, 721 aa, Mycobacterium tuberculosis
MGIALTDDHRELSGVARAFLTSQKVRWAARASLDAAGDARPPFWQNLAELGWLGLHIDERHGGSGYGLSELVVVIEELGRAVAPGLFVPTVIASAVVAKEGTDDQRARLLPALIDGTLTAGVGLDSQVQVTDGVADGEAGIVLGAGLAELLLVAAGDDVLVLERGRKGVSVDVPENFDPTRRSGRVRLDNVRVTTDDILLGAYESALARARTLLAAEAVGGAADCVDSAVAYAKVRQQFGRTIATFQAVKHHCANMLVAAESAIAAVWDAARAAAEDEEQFRLAAAVAAALAFPAYARNAELNIQVHGGIGFTWEHDAHLHLRRALVTVGLFGGDAPVRDVFERTAAGVTRAISLDLPAQAEELRARIRSDAAEIAALEKDAQRDKLIETGYVMPHWPRPWGRAAGAVEQLVIEEEFSAAGIERPDYSITGWVILTLIQHGTPWQIERFVEKALRQQEIWCQLFSEPDAGSDAASVKTRATRVEGGWKINGQKVWTSGAQYCARGLATVRTDPDAPKHAGITTVIIDMLAPGVEVRPLRQITGDSEFNEVFFNDVFVPDEDVVGAPNSGWTVARATLGNERVSIGGSGSYYEAMAAKLVQLVQRRSDAFAGAPIRVGAFLAEDHALRLLNLRRAARSVEGAGPGPEGNITKLKVAEHMIEGAAIAAALWGPEIALLDGPGRVIGRTVMGARGMAIAGGTSEVTRNQIAERILGMPRDPLIS

SEQ ID NO: 31 - DNA, 524 bp, Mycobacterium B3683
atgaccaccggcgacaccgagctgcccgactacaagcgggcccgccgggcccagatcgtcgatgcggcactggatctgctgaagtcacaggactacgagcagatccagatgcgcgatgtcgccgatcacgcccgagtcgcattgggcaccctgtaccgatacttcagctccaaggagcacgtttacgccgcggtcctgatgcagtgggcgcaaccggttttcgccgcggcggaagcggtccgaccggccaccgaacagcaggtccgcgagaagatgcgcggcatcatcaccagcttcgaacgtcggccggcgttcttcaaggtctgcatgctgttgcagaacaccactgacgccaatgcccgcgacctgatggatcgattcgcctccgtcgcccagcgcaccctggccacggacttcgccgccatgggcgaacagggatcggccgacaccgcgatcatggcctggggcatcatctcgaccatgctgtccgcgtccatcctgcgcgacctgccgatggccgacac

SEQ ID NO: 32 - protein, 174 aa, Mycobacterium B3683
MTTGDTELPDYKRARRAQIVDAALDLLKSQDYEQIQMRDVADHARVALGTLYRYFSSKEHVYAAVLMQWAQPVFAAAEAVRPATEQQVREKMRGIITSFERRPAFFKVCMLLQNTTDANARDLMDRFASVAQRTLATDFAAMGEQGSADTAIMAWGIISTMLSASILRDLPMAD

SEQ ID NO: 33 - protein, 211 aa, Nocardia farcinica
MASPSRSQPAAARPATVTTLSEDELSSAAQRERRKRILDATLALASKGGYDAVQMRAVAERADVAVGTLYRYFPSKVHLLVSALAREFEQFESKRKPLAGATPRERMHLLLTQITRMMQRDPLLTEAMTRAFMFADASAAAEVDRVGKVMDRVFARAMNDGEPDERQLAIARVISDVWLSNLVAWLTRRASATDVSDRLELTVDLLLGDKE

SEQ ID NO: 34 - protein, 199 aa, Mycobacterium tuberculosis
MAVLAESELGSEAQRERRKRILDATMAIASKGGYEAVQMRAVADRADVAVGTLYRYFPSKVHLLVSALGREFSRIDAKTDRSAVAGATPFQRLNFMVGKLNRAMQRNPLLTEAMTRAYVFADASAASEVDQVEKLIDSMFARAMANGEPTEDQYHIARVISDVWLSNLLAWLTRRASATDVSKRLDLAVRLLIGDQDSA

SEQ ID NO: 35 - protein, 208 aa, Rhodococcus erythropolis
MMGATLPRIAEVRDAAEPSSDEQRARHVRMLEAAAELGTEKELSRVQMHEVAKRAGVAIGTLYRYFPSKTHLFVAVMVEQIDQIGDSFAKHQVQSANPQDAVYEVLVRATRGLLRRPALSTAMLQSSSTANVATVPDVGKIDRGFRQIILDAAGIENPTEEDNTGLRLLMQLWFGVIQSCLNGRISIPDAEYDIRKGCDLLLVNLSRH

SEQ ID NO: 36 - protein, 218 aa, Streptomyces avermitilis
MPAEAKVEASTGARAARPAVQPASPPLTERQEARRRRILHASAQLASRGGFDAVQMREVAESSQVALGTLYRYFPSKVHLLVATMQAQLEHMHGTLRKKPPAGDTAAERVAETLMRAFRALQREPHLADAMVRALTFADRSVSPEVDQVSRQTTVIILDAMGLDDPTPEQLSAVRVIEHTWHSALITWLSGRASIAQVKIDIETVCRLIDLTEADETP

(19) United States
(12) Patent Application Publication
(10) Pub. No.: US 2011/0191875 A1
(43) Pub. Date: Aug. 4, 2011

(54) COMPOSITIONS AND METHODS FOR MAKING ANDROSTENEDIONES

(75) Inventors: David Nunn, Carlsbad, CA (US); Catherine Pujol, Santee, CA (US); Kelly Chatman, San Diego, CA (US)

(73) Assignee: Verenium Corporation, San Diego, CA (US)

(21) Appl. No.: 12/742,643

(22) PCT Filed: Nov. 13, 2008
(86) PCT No.: PCT/US08/83452

§ 371 Date: Jan. 21, 2011

Related U.S. Application Data
(60) Provisional application No. 60/988,535, filed on Nov. 16, 2007.

(51) Int. Cl. A01K 67/027; A61K 39/40; C12P 33/00; C12P 21/00; C12N 15/00; C12N 11/00; C12N 11/14; C12N 11/02; C12N 11/08; C12N 9/02; C12N 9/10; C12N 5/07; C12N 5/04; C12N 1/21; C12N 1/15; C12N 1/19; C12N 15/63; G01N 33/53; G01N 33/68; C40B 40/06; C40B 40/10; C07K 14/35; C07K 16/12; C07K 16/40; C07K 1/107; C12N 15/52; C12N 15/31; C07H 21/00; A01H 1/00; A01H 5/10; A01H 5/00; C12N 11/16; G06F 19/00

(52) U.S. Cl. 800/15; 424/168.1; 435/52; 435/69.1; 435/440; 435/174; 435/176; 435/177; 435/180; 435/189; 435/193; 435/325; 435/348; 435/375; 435/419; 435/252.3; 435/254.11; 435/254.2; 435/320.1; 436/501; 436/86; 506/16; 506/18; 530/350; 530/387.9; 530/388.26; 530/389.1; 530/402; 536/23.2; 536/23.7; 536/24.32; 536/24.5; 536/25.4; 800/14; 800/16; 800/17; 800/18; 800/278; 800/298; 800/312; 800/317.2; 800/317.3; 800/317.4; 800/320; 800/320.1; 800/320.2; 800/320.3; 800/322; 702/19

(57) ABSTRACT

The invention provides compositions and methods for producing androstenedione (4-androstenedione), of improved purity and for modulating its production, for example by deletion or inactivation of ksdA, cxgA, cxgB, cxgC, or cxgD. The invention also provides methods and compositions, including nucleic acids that encode enzymes, for producing 1,4-androstadiene-3,17-dione (ADD) and related pathway compounds, including 20-(hydroxymethyl)pregna-4-en-3-one and 20-(hydroxymethyl)pregna-1,4-dien-3-one. The compositions of the invention include nucleic acids, probes, vectors, cells, transgenic plants and seeds, transgenic animals, kits and arrays.

### REFERENCE TO SEQUENCE LISTING SUBMITTED VIA EFS-WEB

The entire content of the following electronic submission of the sequence listing via the USPTO EFS-WEB server, as authorized and set forth in MPEP §1730 II.B.2(a)(C), is incorporated herein by reference in its entirety for all purposes. The sequence listing is identified on the electronically filed text file as follows:

| File Name | Date of Creation | Size (bytes) |
|---|---|---|
| 564462016440Seqlist.txt | Nov. 13, 2008 | 120,834 bytes |

### FIELD OF THE INVENTION

This invention generally relates to biology and medicine. The invention provides methods for producing androstenedione (AD, or 4-androstene-3,17-dione), of improved purity and for modulating its production, for example by deletion or inactivation of ksdA, cxgA, cxgB, cxgC, or cxgD genes or gene activity. The invention also provides methods and compositions, including nucleic acids that encode enzymes, for producing 1,4-androstadiene-3,17-dione (ADD) and related pathway compounds, including 20-(hydroxymethyl)pregna-4-en-3-one and 20-(hydroxymethyl)pregna-1,4-dien-3-one.

### BACKGROUND

Androstenedione, also known as 4-androstene-3,17-dione, is a 19-carbon steroid hormone produced in the adrenal glands and the gonads as an intermediate step in the biochemical pathway that produces the androgen testosterone and the estrogens estrone and estradiol.

Androstenedione is the common precursor of male and female sex hormones. Some androstenedione is also secreted into the plasma, and may be converted in peripheral tissues to testosterone and estrogens. Androstenedione originates either from the conversion of dehydroepiandrosterone or from 17-hydroxyprogesterone.

Conversion of dehydroepiandrosterone to androstenedione requires 17, 20 lyase; 17-hydroxyprogesterone requires 17, 20 lyase for its synthesis. Both reactions that produce androstenedione directly or indirectly depend on 17, 20 lyase. Androstenedione is further converted to either testosterone or estrogen. Conversion of androstenedione to testosterone requires the enzyme 17⊖-hydroxysteroid dehydrogenase, while conversion of androstenedione to estrogen (e.g. estrone and estradiol) requires the enzyme aromatase.

Mycobacterium B3683 is a strain of bacteria that can be used to produce androstenedione (AD) from soybean or tall oil phytosterols. In order to produce androstenedione of sufficient purity with this strain, it was previously necessary to use multiple crystallizations to remove contaminating 1,4-androstadiene-3,17-dione (ADD), 20-(hydroxymethyl)pregna-4-en-3-one (referred to here as compound X1) and 20-(hydroxymethyl)pregna-1,4-dien-3-one (referred to here as compound X2). This protocol can be cost-prohibitive.

Known strains used for sterol conversions generated by conventional mutagenesis, e.g., as Marshek (1972) Applied Microbiology 23(1):72-77, do not specifically delete or knock-out genes that produce the contaminating compounds ADD, X1 and X2.

In earlier pilot-scale experiments using Mycobacterium B3683 (Marshek (1972) supra) for the production of AD, the large amounts of ADD and compounds X1 and X2 produced limited the economic utility of this process due to the high cost of removing these contaminating compounds by multiple crystallizations. Therefore, there is a need to economically produce AD with a significant improvement in purity.

### SUMMARY OF THE INVENTION

This invention provides a method, including an in vivo method, for making androstenedione (4-androstene-3,17-dione, or AD) comprising specific inactivation of genes that produce the contaminating compounds 1,4-androstadiene-3,17-dione (ADD), compound 20-(hydroxymethyl)pregna-4-en-3-one (referred to as compound X1) and 20-(hydroxymethyl)pregna-1,4-dien-3-one (referred to as compound X2). In one embodiment, the invention provides a relatively pure solution of androstenedione (AD) substantially without the impurities ADD, X1 and X2.

The invention also provides methods and compositions, including nucleic acids that encode enzymes, for producing 1,4-androstadiene-3,17-dione (ADD) and related pathway compounds, including 20-(hydroxymethyl)pregna-4-en-3-one and 20-(hydroxymethyl)pregna-1,4-dien-3-one.

The invention also provides a prokaryotic system, e.g., a Mycobacterial system, for making AD lacking active genes that produce the contaminating compounds ADD, X1 and X2. In alternative embodiments, in the prokaryotic systems and cells of the invention only these relevant genes are affected, i.e., only the activity of the genes that produce the “contaminating”compounds ADD, X1 and X2 are decreased or eliminated (“contaminating” in the context where the objective is to make more pure, or relatively pure, or substantially pure, AD). In alternative embodiments, the activity of the genes that produce the “contaminating” compounds ADD, X1 and X2 are decreased or eliminated on a protein and/or a nucleic acid, e.g., a gene or transcript (mRNA, message) level. For example, the genes that produce the contaminating compounds ADD, X1 and X2 can be knocked out partially or completely; the transcriptional control sequence (e.g., promoters, enhancers) for the genes that produce the contaminating compounds ADD, X1 and X2 genes can be partially or completely disabled; the trans-acting factors that turn on the transcription of the genes that produce the contaminating compounds ADD, X1 and X2 genes via their transcriptional control sequences (e.g., promoters, enhancers) can be partially or completely disabled; the genes that produce the contaminating compounds ADD, X1 and X2 genes can be mutated, e.g., by base changes, insertional disruptions, deletions and the like; the processing or expression of their transcripts can be partially or completely blocked, and/or the activity of the polypeptide enzymes they express can be partially or completely blocked. In one embodiment, genes that produce the contaminating compounds ADD, X1 and X2 that the invention targets comprise or consist of ksdA, cxgA, cxgB, cxgC and/or cxgD. Thus, in alternative embodiments, the invention provide methods and compositions (e.g., cells, prokaryotic systems) wherein the enzyme coding sequences of ksdA, cxgA, cxgB, cxgC and/or cxgD, are modified (e.g., disabled), their transcriptional control sequences are modified (e.g., inhibited), their trans-acting factors are modified (e.g., disabled), their transcripts (mRNAs) are modified and/or the enzymes they encode are modified.

In alternative embodiments, the invention provides compositions and methods for producing androstenedione (AD) of improved purity (e.g., substantially pure) and for modulating AD production, for example by deletion or inactivation of the genes ksdA, cxgA, cxgB, cxgC, or cxgD; their transcriptional control sequences, trans-acting factors or transcripts and/or the enzymes they encode.

The invention also provides isolated, synthetic or recombinant nucleic acids that encode proteins for producing 1,4-androstadiene-3,17-dione (ADD) and the related pathway compounds X1 and X2, including expression vehicles (e.g., vectors, plasmids) and cells that comprise these nucleic acids.

In alternative embodiments, the methods of the invention are designed to avoid the introduction of random mutations throughout a host organism (for the expression and manufacture of AD), e.g., a prokaryotic host cell, e.g., a Mycobacteria, which may lead to reduced performance or robustness of the host cell.

The invention provides for the first time combinations of nucleic acids, e.g., genes, and combinations of genes in host cells, and the resultant encoded recombinant proteins required for the production of the impurities described above, i.e., the contaminating compounds ADD, X1 and X2.

In alternative embodiments, the nucleic acids, e.g., genes, of the invention also can be used to produce or to increase production of ADD, X1 and X2, which also have commercial value as steroidal intermediates.

The invention provides isolated, synthetic or recombinant nucleic acids comprising:

(a) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:1, and having a KsdA polypeptide or a 3-ketosteroid-Δ1-dehydrogenase activity;

(b) a nucleic acid sequence encoding a polypeptide having an amino acid sequence as set forth in SEQ ID NO:2, and having a KsdA polypeptide or 3-ketosteroid-Δ1-dehydrogenase activity, and enzymatically active fragments thereof;

(c) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:9, and having a CxgA polypeptide or an acetyl CoA-acetyltransferase/thiolase activity;

(d) a nucleic acid sequence encoding a polypeptide having an amino acid sequence as set forth in SEQ ID NO:10 or SEQ ID NO:11, and having a CxgA polypeptide or an acetyl CoA-acetyltransferase/thiolase activity, and enzymatically active fragments thereof;

(e) a nucleic acid sequence encoding a polypeptide having at least about 75%, 76%, 77s %, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:17, and having a CxgB polypeptide or a DNA-binding protein activity;

(f) a nucleic acid sequence encoding a polypeptide having an amino acid sequence as set forth in SEQ ID NO:18, and having a CxgB polypeptide or a DNA-binding protein activity, and DNA-binding active fragments thereof;

(g) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:24, and having a CxgC polypeptide or a DNA-binding protein activity;

(h) a nucleic acid sequence encoding a polypeptide having an amino acid sequence as set forth in SEQ ID NO:25, and having a CxgC polypeptide or an acyl-CoA dehydrogenase/FadE activity, and enzymatically active fragments thereof;

(i) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:31, and having a CxgD polypeptide or a TetR-like regulatory protein/KstR activity;

(j) a nucleic acid sequence encoding a polypeptide having an amino acid sequence as set forth in SEQ ID NO:32, and having a CxgD polypeptide or a TetR-like regulatory protein/KstR activity, and enzymatically active fragments thereof;

(k) the nucleic acid of any of (a) to (j), wherein the sequence identities are determined by analysis with a sequence comparison algorithm or by a visual inspection;

(l) the nucleic acid of (k), wherein the sequence comparison algorithm is a BLAST version 2.2.2 algorithm where a filtering setting is set to blastall-p blastp-d “nr pataa”-F F, and all other options are set to default, or a FASTA version 3.0t78, with the default parameters;

(m) a nucleic acid sequence that hybridizes under stringent conditions to a nucleic acid consisting of SEQ ID NO:1, SEQ ID NO:9, SEQ ID NO:17, SEQ ID NO:24 and/or SEQ ID NO:31, and the nucleic acid encodes a polypeptide having a KsdA polypeptide or 3-ketosteroid-Δ1-dehydrogenase activity, a CxgA polypeptide or an acetyl CoA-acetyltransferase/thiolase activity, a CxgB polypeptide or a DNA-binding protein activity, a CxgC polypeptide or an acyl-CoA dehydrogenase/FadE activity, or a CxgD polypeptide or a TetR-like regulatory protein/KstR activity, respectively,

wherein the stringent conditions include a wash step comprising a wash in 0.2×SSC at a temperature of about 65° C. for about 15 minutes;

(n) the nucleic acid of any of (a) to (m) encoding a polypeptide lacking a signal sequence or proprotein sequence, or lacking a homologous promoter sequence;

(o) the nucleic acid of any of (a) to (n) further comprising a sequence encoding a heterologous amino acid sequence, or the nucleic acid further comprises a heterologous nucleotide sequence;

(p) the nucleic acid of (o) wherein the heterologous amino acid sequence comprises, or consists of a sequence encoding a heterologous (leader) signal sequence, or a tag or an epitope, or the heterologous nucleotide sequence comprises a heterologous promoter sequence;

(q) the nucleic acid of (o) or (p), wherein the heterologous nucleotide sequence encodes a heterologous (leader) signal sequence comprising or consisting of an N-terminal and/or C-terminal extension for targeting to an endoplasmic reticulum (ER) or endomembrane, or to a bacterial endoplasmic reticulum (ER) or endomembrane system, or the heterologous sequence encodes a restriction site;

(r) the nucleic acid of (p), wherein the heterologous promoter sequence comprises or consists of a constitutive or inducible promoter, or a cell type specific promoter, or a plant specific promoter, or a bacteria specific promoter, or a Mycobacterium specific promoter;

(s) the nucleic acid of any of (a) to (r), wherein the enzyme activity is thermotolerant; or

(t) a nucleic acid sequence completely complementary to the nucleotide sequence of any of (a) to (s).

The invention provides probes for isolating or identifying a KsdA, CxgA, CxgB, CxgC or CxgD-encoding nucleic acid comprising a nucleic acid of the invention.

The invention provides vectors, expression cassettes or cloning vehicles: (a) comprising the nucleic acid (polynucleotide) sequence of the invention; or, (b) the vector, expression cassette or cloning vehicle of (a) comprising or contained in a viral vector, a plasmid, a phage, a phagemid, a cosmid, a fosmid, a bacteriophage, an artificial chromosome, an adenovirus vector, a retroviral vector or an adeno-associated viral vector; or, a bacterial artificial chromosome (BAC), a plasmid, a bacteriophage P1-derived vector (PAC), a yeast artificial chromosome (YAC), or a mammalian artificial chromosome (MAC).

The invention provides host cells or a transformed cells: (a) comprising a nucleic acid (polynucleotide) sequence of the invention, or a vector, expression cassette or cloning vehicle of the invention; or, (b) the host cell or a transformed cell of (a), wherein the cell is a bacterial cell, a mammalian cell, a fungal cell, a yeast cell, an insect cell or a plant cell.

The invention provides transgenic non-human animals: (a) comprising a nucleic acid (polynucleotide) sequence of the invention; a vector, expression cassette or cloning vehicle of the invention; or a host cell or a transformed cell of the invention; or (b) the transgenic non-human animal of (a), wherein the animal is a mouse, a rat, a goat, a rabbit, a sheep, a pig or a cow.

The invention provides transgenic plants or seeds: (a) comprising a nucleic acid (polynucleotide) sequence of the invention; a vector, expression cassette or cloning vehicle of the invention; or a host cell or a transformed cell of the invention; (b) the transgenic plant of (a), wherein the plant is a corn plant, a sorghum plant, a potato plant, a tomato plant, a wheat plant, an oilseed plant, a rapeseed plant, a soybean plant, a rice plant, a barley plant, a grass, a cottonseed, a palm, a sesame plant, a peanut plant, a sunflower plant or a tobacco plant; the transgenic seed of (a), wherein the seed is a corn seed, a wheat kernel, an oilseed, a rapeseed, a soybean seed, a palm kernel, a sunflower seed, a sesame seed, a rice, a barley, a peanut, a cottonseed, a palm, a peanut, a sesame seed, a sunflower seed or a tobacco plant seed.

The invention provides antisense oligonucleotides comprising a nucleic acid sequence complementary to or capable of hybridizing under stringent conditions to the nucleic acid (polynucleotide) sequence of the invention.

The invention provides methods of inhibiting the translation of a message (mRNA) in a cell comprising administering to the cell or expressing in the cell an antisense oligonucleotide comprising the nucleic acid (polynucleotide) sequence of the invention.

The invention provides isolated, synthetic or recombinant polypeptides comprising:

(a) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:2, and enzymatically active fragments thereof, and having a ksdA polypeptide or a 3-ketosteroid-Δ1-dehydrogenase activity;

(b) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:10 or SEQ ID NO:11, and enzymatically active fragments thereof, and having a cxgA polypeptide or an acetyl CoA-acetyltransferase/thiolase activity;

(c) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:18, and enzymatically active fragments thereof, and having a cxgB polypeptide or a DNA-binding protein activity;

(d) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:25, and enzymatically active fragments thereof, and having a cxgC polypeptide or a DNA-binding protein activity;

(e) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:32, and enzymatically active fragments thereof, and having a cxgD polypeptide or a TetR-like regulatory protein/KstR activity;

(f) the polypeptide of any of (a) to (e), wherein the sequence identities are determined by analysis with a sequence comparison algorithm or by a visual inspection;

(g) the polypeptide of (f), wherein the sequence comparison algorithm is a BLAST version 2.2.2 algorithm where a filtering setting is set to blastall-p blastp-d “nr pataa”-F F, and all other options are set to default, or a FASTA version 3.0t78, with the default parameters;

(h) a polypeptide encoded by the nucleic acid of any of the invention;

(i) the polypeptide of any of (a) to (h), lacking a signal sequence or proprotein sequence;

(j) the polypeptide of any of (a) to (i) further comprising a heterologous amino acid sequence;

(k) the polypeptide of (j) wherein the heterologous amino acid sequence comprises, or consists of, a heterologous (leader) signal sequence, or a tag or an epitope;

(l) the polypeptide of (j), wherein the heterologous (leader) signal sequence comprises or consists of an N-terminal and/or C-terminal extension for targeting to an endoplasmic reticulum (ER) or endomembrane, or to a bacterial endoplasmic reticulum (ER) or endomembrane system;

(m) the polypeptide of any of (a) to (l), wherein the enzyme activity is thermotolerant; or

(n) the polypeptide of any of (a) to (m), wherein the polypeptide is glycosylated, or the polypeptide comprises at least one glycosylation site, (ii) the polypeptide of (i) wherein the glycosylation is an N-linked glycosylation or an O-linked glycosylation; (iii) the polypeptide of (i) or (ii) wherein the polypeptide is glycosylated after being expressed in a yeast cell.

The invention provides protein preparations comprising the polypeptide of the invention, wherein the protein preparation comprises a liquid, a solid or a gel.

The invention provides heterodimers: (a) comprising a polypeptide of the invention and a second domain; or (b) the heterodimer of (a), wherein the second domain is a polypeptide and the heterodimer is a fusion protein, or the second domain is an epitope or a tag. The invention provides homodimers comprising a polypeptide of the invention.

The invention provides immobilized polypeptides: (a) wherein the polypeptide comprises a polypeptide of the invention; or, (b) the immobilized polypeptide of (a), wherein the polypeptide is immobilized on a cell, a metal, a resin, a polymer, a ceramic, a glass, a microelectrode, a graphitic particle, a bead, a gel, a plate, an array or a capillary tube.

The invention provides isolated, synthetic or recombinant antibodies: (a) that specifically binds to a polypeptide of the invention; or, (b) the isolated, synthetic or recombinant antibody of (a), wherein the antibody is a monoclonal or a polyclonal antibody, or antigen binding fragment thereof. The invention provides hybridomas comprising an antibody of the invention.

The invention provides arrays comprising an immobilized nucleic acid, polypeptide and/or antibody of the invention, or a combination of a nucleic acid, polypeptide (including isolated, synthetic or recombinant forms, and fusion proteins) and/or antibody of the invention.

The invention provides methods of isolating or identifying a polypeptide having a KsdA, CxgA, CxgB, CxgC or CxgD activity, comprising:

(a) providing the antibody of the invention;

(b) providing a sample comprising polypeptides; and

(c) contacting the sample of step (b) with the antibody of step (a) under conditions wherein the antibody can specifically bind to the polypeptide, thereby isolating or identifying a polypeptide having a KsdA, CxgA, CxgB, CxgC or CxgD activity.

The invention provides methods of making an anti-KsdA, CxgA, CxgB, CxgC or CxgD antibody comprising administering to a non-human animal:

(a) the KsdA, CxgA, CxgB, CxgC or CxgD-encoding nucleic acid (polynucleotide) sequence of the invention in an amount sufficient to generate a humoral immune response, thereby making an anti-KsdA, CxgA, CxgB, CxgC or CxgD antibody; or

(b) the polypeptide of the invention in an amount sufficient to generate a humoral immune response, thereby making an anti-KsdA, CxgA, CxgB, CxgC or CxgD antibody.

The invention provides methods of producing a recombinant polypeptide comprising:

(A) (a) providing a nucleic acid operably linked to a promoter, wherein the nucleic acid comprises the nucleic acid (polynucleotide) sequence of the invention; and (b) expressing the nucleic acid of step (a) under conditions that allow expression of the polypeptide, thereby producing a recombinant polypeptide; or

(B) the method of (A), further comprising transforming a host cell with the nucleic acid of step (a) followed by expressing the nucleic acid of step (a), thereby producing a recombinant polypeptide in a transformed cell.

The invention provides methods for identifying a polypeptide having KsdA, CxgA, CxgB, CxgC or CxgD activity comprising:

(a) providing the polypeptide of the invention;

(b) providing a KsdA, CxgA, CxgB, CxgC or CxgD binding protein or substrate; and

(c) contacting the polypeptide with the substrate of step (b) and detecting a decrease in the amount of substrate or an increase in the amount of a reaction product, wherein a decrease in the amount of the substrate or an increase in the amount of the reaction product detects a polypeptide having a KsdA, CxgA, CxgB, CxgC or CxgD activity.

The invention provides methods for identifying a KsdA, CxgA, CxgB, CxgC or CxgD binding protein or substrate comprising:

(a) providing a KsdA, CxgA, CxgB, CxgC or CxgD polypeptide of the invention;

(b) providing a test binding protein or substrate; and

(c) contacting the KsdA, CxgA, CxgB, CxgC or CxgD polypeptide of step (a) with the test binding protein or substrate of step (b) and detecting a decrease in the amount of binding protein or substrate or an increase in the amount of reaction product, wherein a decrease in the amount of the substrate or an increase in the amount of a reaction product identifies the test substrate as a KsdA, CxgA, CxgB, CxgC or CxgD binding protein or substrate.

The invention provides methods of determining whether a test compound specifically binds to a KsdA, CxgA, CxgB, CxgC or CxgD polypeptide comprising:

(a) expressing a nucleic acid or a vector comprising the nucleic acid under conditions permissive for translation of the nucleic acid to a polypeptide, wherein the nucleic acid has the nucleic acid (polynucleotide) sequence of the invention;

(b) providing a test compound;

(c) contacting the KsdA, CxgA, CxgB, CxgC or CxgD polypeptide with the test compound; and

(d) determining whether the test compound of step (b) specifically binds to the KsdA, CxgA, CxgB, CxgC or CxgD polypeptide.

The invention provides methods of determining whether a test compound specifically binds to a KsdA, CxgA, CxgB, CxgC or CxgD polypeptide comprising:

(a) providing the KsdA, CxgA, CxgB, CxgC or CxgD polypeptide of the invention;

(b) providing a test compound;

(c) contacting the polypeptide with the test compound; and

(d) determining whether the test compound of step (b) specifically binds to the ksdA, cxgA, cxgB, cxgC or cxgD polypeptide.

The invention provides methods for identifying a modulator of a KsdA, CxgA, CxgB, CxgC or CxgD polypeptide comprising:

(A) (a) providing the KsdA, CxgA, CxgB, CxgC or CxgD polypeptide of the invention;

(b) providing a test compound;

(c) contacting the polypeptide of step (a) with the test compound of step (b) and measuring an activity of the KsdA, CxgA, CxgB, CxgC or CxgD polypeptide, wherein a change in the KsdA, CxgA, CxgB, CxgC or CxgD activity measured in the presence of the test compound compared to the activity in the absence of the test compound provides a determination that the test compound modulates the KsdA, CxgA, CxgB, CxgC or CxgD activity;

(B) the method of (A), wherein the KsdA, CxgA, CxgB, CxgC or CxgD activity is measured by providing a KsdA, CxgA, CxgB, CxgC or CxgD substrate and detecting a decrease in the amount of the substrate or an increase in the amount of a reaction product, or, an increase in the amount of the substrate or a decrease in the amount of a reaction product;

(c) the method of (B), wherein a decrease in the amount of the substrate or an increase in the amount of the reaction product with the test compound as compared to the amount of substrate or reaction product without the test compound identifies the test compound as an activator of KsdA, CxgA, CxgB, CxgC or CxgD activity; or,

(d) the method of (B), wherein an increase in the amount of the substrate or a decrease in the amount of the reaction product with the test compound as compared to the amount of substrate or reaction product without the test compound identifies the test compound as an inhibitor of KsdA, CxgA, CxgB, CxgC or CxgD activity.

The invention provides computer systems comprising:

(a) a processor and a data storage or a machine readable memory device wherein said data storage device has stored thereon a polypeptide sequence or a nucleic acid sequence, wherein the polypeptide sequence comprises the polypeptide (amino acid) sequence of the invention, a polypeptide encoded by the nucleic acid (polynucleotide) sequence of the invention;

(b) the computer system of (a), further comprising a sequence comparison algorithm and a data storage device or machine readable memory device having at least one reference sequence stored thereon;

(c) the computer system of (b), wherein the sequence comparison algorithm comprises a computer program that indicates polymorphisms; or

(d) the computer system of any of (a) to (c), further comprising an identifier that identifies one or more features in said sequence.

The invention provides computer readable medium (media) or machine readable memory devices having stored thereon a polypeptide sequence or a nucleic acid sequence, wherein the polypeptide sequence comprises a polypeptide (amino acid) sequence of the invention; or, a polypeptide encoded by the nucleic acid (polynucleotide) sequence of the invention.

The invention provides methods for identifying a feature in a sequence comprising: (a) reading the sequence using a computer program functionally saved (embedded in) a computer or a machine readable memory device, wherein the computer program identifies one or more features in a sequence, wherein the sequence comprises a polypeptide sequence or a nucleic acid sequence, wherein the polypeptide sequence comprises the polypeptide (amino acid) sequence of the invention; a polypeptide encoded by the nucleic acid (polynucleotide) sequence of the invention; and, (b) identifying one or more features in the sequence with the computer program.
The invention provides methods for isolating or recovering a nucleic acid encoding a polypeptide with a KsdA, CxgA, CxgB, CxgC or CxgD activity from a sample comprising:

(A) (a) providing a polynucleotide probe comprising the nucleic acid (polynucleotide) sequence of the invention;

(b) isolating a nucleic acid from the sample or treating the sample such that nucleic acid in the sample is accessible for hybridization to a polynucleotide probe of step (a);

(c) combining the isolated nucleic acid or the treated sample of step (b) with the polynucleotide probe of step (a); and

(d) isolating a nucleic acid that specifically hybridizes with the polynucleotide probe of step (a), thereby isolating or recovering a nucleic acid encoding a polypeptide with a KsdA, CxgA, CxgB, CxgC or CxgD activity from a sample;

(B) the method of (A), wherein the sample is or comprises an environmental sample;

(C) the method of (B), wherein the environmental sample is or comprises a water sample, a liquid sample, a soil sample, an air sample or a biological sample; or

(D) the method of (C), wherein the biological sample is derived from a bacterial cell, a protozoan cell, an insect cell, a yeast cell, a plant cell, a fungal cell or a mammalian cell.

The invention provides methods of generating a variant of a nucleic acid encoding a polypeptide with a KsdA, CxgA, CxgB, CxgC or CxgD activity comprising:

(A) (a) providing a template nucleic acid comprising the nucleic acid (polynucleotide) sequence of the invention; and

(b) modifying, deleting or adding one or more nucleotides in the template sequence, or a combination thereof, to generate a variant of the template nucleic acid.

(B) the method of (A), further comprising expressing the variant nucleic acid to generate a variant KsdA, CxgA, CxgB, CxgC or CxgD polypeptide;

(C) the method of (A) or (B), wherein the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) and a combination thereof;

(D) the method of any of (A) to (C), wherein the modifications, additions or deletions are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof;

(E) the method of any of (A) to (D), wherein the method is iteratively repeated until a (variant) KsdA, CxgA, CxgB, CxgC or CxgD polypeptide having an altered or different (variant) activity, or an altered or different (variant) stability from that of a polypeptide encoded by the template nucleic acid is produced, or an altered or different (variant) secondary structure from that of a polypeptide encoded by the template nucleic acid is produced, or an altered or different (variant) post-translational modification from that of a polypeptide encoded by the template nucleic acid is produced;

(F) the method of (E), wherein the variant KsdA, CxgA, CxgB, CxgC or CxgD polypeptide is thermotolerant, and retains some activity after being exposed to an elevated temperature;

(G) the method of (E), wherein the variant KsdA, CxgA, CxgB, CxgC or CxgD polypeptide has increased glycosylation as compared to the KsdA, CxgA, CxgB, CxgC or CxgD activity encoded by a template nucleic acid;

(H) the method of (E), wherein the variant KsdA, CxgA, CxgB, CxgC or CxgD polypeptide has a KsdA, CxgA, CxgB, CxgC or CxgD activity under a high temperature, wherein the KsdA, CxgA, CxgB, CxgC or CxgD polypeptide encoded by the template nucleic acid is not active under the high temperature;

(I) the method of any of (A) to (H), wherein the method is iteratively repeated until a KsdA, CxgA, CxgB, CxgC or CxgD polypeptide coding sequence having an altered codon usage from that of the template nucleic acid is produced; or

(J) the method of any of (A) to (H), wherein the method is iteratively repeated until a ksdA, cxgA, cxgB, cxgC or cxgD gene having higher or lower level of message expression or stability from that of the template nucleic acid is produced.

The invention provides methods for modifying codons in a nucleic acid encoding a polypeptide with a KsdA, CxgA, CxgB, CxgC or CxgD activity to increase its expression in a host cell, the method comprising:

(a) providing a nucleic acid encoding a polypeptide with a KsdA, CxgA, CxgB, CxgC or CxgD activity comprising the nucleic acid (polynucleotide) sequence of the invention; and,

(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying codons in a nucleic acid encoding a KsdA, CxgA, CxgB, CxgC or CxgD polypeptide, the method comprising:

(a) providing a nucleic acid encoding a polypeptide with a KsdA, CxgA, CxgB, CxgC or CxgD activity comprising the nucleic acid (polynucleotide) sequence of the invention; and,

(b) identifying a codon in the nucleic acid of step (a) and replacing it with a different codon encoding the same amino acid as the replaced codon, thereby modifying codons in a nucleic acid encoding a KsdA, CxgA, CxgB, CxgC or CxgD polypeptide.

The invention provides methods for modifying codons in a nucleic acid encoding a KsdA, CxgA, CxgB, CxgC or CxgD polypeptide to increase its expression in a host cell, the method comprising:

(a) providing a nucleic acid encoding a KsdA, CxgA, CxgB, CxgC or CxgD polypeptide comprising the nucleic acid (polynucleotide) sequence of the invention; and,

(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides methods for modifying a codon in a nucleic acid encoding a polypeptide having a KsdA, CxgA, CxgB, CxgC or CxgD activity to decrease its expression in a host cell, the method comprising:

(A) (a) providing a nucleic acid encoding a KsdA, CxgA, CxgB, CxgC or CxgD polypeptide comprising the nucleic acid (polynucleotide) sequence of the invention; and

(b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell; or

(B) the method of (A), wherein the host cell is a bacterial cell, a fungal cell, an insect cell, a yeast cell, a plant cell or a mammalian cell.

The invention provides methods for increasing the thermotolerance or thermostability of a KsdA, CxgA, CxgB, CxgC or CxgD polypeptide, the method comprising glycosylating a KsdA, CxgA, CxgB, CxgC or CxgD polypeptide, wherein the polypeptide comprises at least thirty contiguous amino acids of the polypeptide of the invention, or a polypeptide encoded by the nucleic acid (polynucleotide) sequence of the invention, thereby increasing the thermotolerance or thermostability of the KsdA, CxgA, CxgB, CxgC or CxgD polypeptide.

The invention provides methods for overexpressing a recombinant KsdA, CxgA, CxgB, CxgC or CxgD polypeptide in a cell comprising expressing a vector comprising the nucleic acid (polynucleotide) sequence of the invention, wherein overexpression is effected by use of a high activity promoter, a dicistronic vector or by gene amplification of the vector.

The invention provides methods of making a transgenic plant comprising:

(A) (a) introducing a heterologous nucleic acid sequence into the cell, wherein the heterologous nucleic sequence comprises the nucleic acid (polynucleotide) sequence of the invention, thereby producing a transformed plant cell; and (b) producing a transgenic plant from the transformed cell;

(B) the method of (A), wherein the step (A)(a) further comprises introducing the heterologous nucleic acid sequence by electroporation or microinjection of plant cell protoplasts; or

(C) the method of (C), wherein the step (A)(a) comprises introducing the heterologous nucleic acid sequence directly to plant tissue by DNA particle bombardment or by using an Agrobacterium tumefaciens host.

The invention provides methods of expressing a heterologous nucleic acid sequence in a plant cell comprising the following steps:

(a) transforming the plant cell with a heterologous nucleic acid sequence operably linked to a promoter, wherein the heterologous nucleic sequence comprises the nucleic acid (polynucleotide) sequence of the invention; and

(b) growing the plant under conditions wherein the heterologous nucleic acids sequence is expressed in the plant cell.

The invention provides methods (processes) for modulating the production of androstenedione (AD, or 4-androstenedione), androstadienedione (ADD, or 1,4-androstadiene-3,17-dione), 20-(hydroxymethyl)pregna-4-en-3-one and/or 20-(hydroxymethyl)pregna-1,4-dien-3-one in a cell, comprising:

(a) (i) over- or underexpressing any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell, or (ii) deleting expression of any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell;

(b) the process of (a) wherein the cell is a prokaryotic cell or a eukaryotic cell;

(c) the process of (b) wherein the prokaryotic cell is a bacterial cell, or the eukaryotic cell is a yeast or fungal cell;

(d) the process of (c), wherein the bacterial cell is a member of the genus Actinobacteria, or a member of the family Mycobacteriaceae;

(e) the process of (d), wherein the member of the family Mycobacteriaceae is a Mycobacterium strain designated B3683 and/or B3805, or Mycobacterium ATCC 29472;

(f) the process of any of (a) to (e), wherein the any one, or several of, or all of KsdA-, CxgA-, CxgB-, CxgC- and/or CxgD-encoding nucleic acids are over- or underexpressed by a process comprising deleting, mutating or disrupting a transcriptional control sequence for a ksdA, cxgA, cxgB, cxgC and/or cxgD gene,

wherein the deleting, mutating or disrupting of the transcriptional control sequence results in the overexpression and/or the underexpression of the ksdA, cxgA, cxgB, cxgC and/or cxgD gene, and/or overexpression and/or the underexpression of the KsdA-, CxgA-, CxgB-, CxgC- and/or CxgD polypeptide-encoding message (mRNA);

(g) the process of (f), wherein the transcriptional control sequence is a promoter and/or an enhancer;

(h) the process of any of (a) to (e), wherein the any one, or several of, or all of KsdA-, CxgA-, CxgB-, CxgC- and/or CxgD-encoding nucleic acids are over- or underexpressed by a process comprising deleting, mutating or disrupting a trans-acting factor that regulates transcription of a ksdA, cxgA, cxgB, cxgC and/or cxgD gene,

wherein the deleting, mutating or disrupting of the trans-acting factor results in the overexpression and/or the underexpression of the ksdA, cxgA, cxgB, cxgC and/or cxgD gene;

(i) the process of any of (a) to (e), wherein the any one, or several of, or all of KsdA-, CxgA-, CxgB-, CxgC- and/or CxgD-encoding nucleic acids are over- or underexpressed by a process comprising upregulating, deleting, mutating or disrupting a message (mRNA) of a KsdA-, CxgA-, CxgB-, CxgC- and/or CxgD-encoding nucleic acid,

wherein the upregulating, deleting, mutating or disrupting of the message (mRNA) results in the overexpression and/or the underexpression of the KsdA-, CxgA-, CxgB-, CxgC- and/or CxgD polypeptides;

(j) the process of (i), wherein the expression of a message (mRNA) of a KsdA-, CxgA-, CxgB-, CxgC- and/or CxgD-encoding nucleic acid is deleted or disrupted by an antisense, ribozyme and/or RNAi specific for a message (mRNA) of a KsdA-, CxgA-, CxgB-, CxgC- and/or CxgD-encoding nucleic acid;

(k) the process of any of (a) to (e), wherein the any one, or several of, or all of the KsdA-, CxgA-, CxgB-, CxgC- and/or CxgD polypeptides in the cell are over- or underexpressed by addition of an inhibitor or activator of the activity of the KsdA-, CxgA-, CxgB-, CxgC- and/or CxgD polypeptide;

(l) the process of (k), wherein the inhibitor or activator of the activity of the KsdA-, CxgA-, CxgB-, CxgC- and/or CxgD polypeptide is a small molecule or an antibody inhibitor or activator of the activity of the KsdA-, CxgA-, CxgB-, CxgC- and/or CxgD polypeptide;

(m) the process of any of (a) to (l), wherein the KsdA-, CxgA-, CxgB-, CxgC- and/or CxgD-encoding nucleic acid comprises a nucleic acid of the invention; or

(n) the process of any of (a) to (l), wherein the KsdA-, CxgA-, CxgB-, CxgC- and/or CxgD polypeptide comprises a polypeptide of the invention.

The invention provides cell-based processes (methods) for producing an androstenedione (AD, or 4-androstene-3,17-dione) of relative purity, or substantially free of androstadienedione (ADD, or 1,4-androstadiene-3,17-dione), 20-(hydroxymethyl)pregna-4-en-3-one and/or 20-(hydroxymethyl)pregna-1,4-dien-3-one, comprising

(a) (i) making a cell that underexpresses (as compared to a wild type cell) or does not express any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell; and, (ii) culturing the cell under conditions wherein the androstenedione is produced,

wherein underexpressing the KsdA-, CxgA-, CxgB-, CxgC- and/or CxgD-encoding nucleic acids and/or KsdA-, CxgA-, CxgB-, CxgC- and/or CxgD polypeptides in the cell results production of an androstenedione (AD) of relative purity, or substantially free of androstadienedione (ADD), 20-(hydroxymethyl)pregna-4-en-3-one and/or 20-(hydroxymethyl)pregna-1,4-dien-3-one; or

(b) the process of (a), wherein the underexpression of the KsdA-, CxgA-, CxgB-, CxgC- and/or CxgD-encoding nucleic acids and/or the KsdA-, CxgA-, CxgB-, CxgC- and/or CxgD polypeptides in the cell is made by practicing a method of the invention;

(c) the process of (a) or (b), wherein the cell underexpresses a KsdA-, CxgA-, CxgB-, CxgC- and/or CxgD-encoding nucleic acid (as compared to a wild type or unmanipulated cell) by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more;

(d) the process of (a) or (b), wherein the cell produces (generates) an androstenedione (AD) of relative greater purity, or substantially free of androstadienedione (ADD), 20-(hydroxymethyl)pregna-4-en-3-one and/or 20-(hydroxymethyl)pregna-1,4-dien-3-one by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;

(e) the process of any of (a) to (d), wherein the cell produces at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more % fewer (lesser amounts of) impurities in the AD synthesis process; or

(f) the process of (e), wherein the fewer impurities comprise fewer (lesser amounts of) androstadienedione (ADD), 20-(hydroxymethyl)pregna-4-en-3-one and/or 20-(hydroxymethyl)pregna-1,4-dien-3-one.

The invention provides cell-based processes (methods) for producing an androstenedione (AD, or 4-androstene-3,17-dione) of relative purity, or substantially free of androstadienedione (ADD, or 1,4-androstadiene-3,17-dione), 20-(hydroxymethyl)pregna-4-en-3-one and/or 20-(hydroxymethyl)pregna-1,4-dien-3-one, comprising

(a) (i) making a cell that underexpresses (as compared to a wild type or unmanipulated cell) or does not express any one, or several of, or all KsdA-, CxgA-, CxgB-, CxgC- and/or CxgD polypeptides in the cell; and, (ii) culturing the cell under conditions wherein androstenedione is produced,

wherein underexpressing or inhibiting the activity of the KsdA-, CxgA-, CxgB-, CxgC- and/or CxgD polypeptides in the cell results production of an androstenedione (AD) of relative purity, or substantially free of androstadienedione (ADD), 20-(hydroxymethyl) pregna-4-en-3-one and/or 20-(hydroxymethyl)pregna-1,4-dien-3-one;

(b) the process of (a), wherein the underexpression of or inhibition of activity of the KsdA-, CxgA-, CxgB-, CxgC- and/or CxgD polypeptides in the cell is by practicing the method of the invention;

(c) the process of (a) or (b), wherein the cell underexpresses a KsdA-, CxgA-, CxgB-, CxgC- and/or CxgD polypeptide (as compared to a wild type or unmanipulated cell) by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;

(d) the process of (a) or (b), wherein the cell underproduces an androstenedione (AD) of relative purity, or substantially free of androstadienedione (ADD), 20-(hydroxymethyl) pregna-4-en-3-one and/or 20-(hydroxymethyl)pregna-1,4-dien-3-one by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;

(e) the process of any of (a) to (d), wherein the cell produces at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more % fewer (lesser amounts of) impurities in the AD synthesis process; or

(f) the process of (e), wherein the fewer impurities comprise fewer (lesser amounts of) androstadienedione (ADD), 20-(hydroxymethyl)pregna-4-en-3-one and/or 20-(hydroxymethyl)pregna-1,4-dien-3-one.

The invention provides kits comprising (a) a nucleic acid of the invention; a probe of the invention; a vector, expression cassette or cloning vehicle of the invention; or, a host cell or a transformed cell of the invention; or (b) the kit of (a), further comprising instructions for practicing any one of the methods of the invention.

The invention provides kits comprising (a) a polypeptide of the invention; an antibody or hybridoma of the invention; an array of the invention; a heterodimer of the invention, or (b) the kit of (a), further comprising instructions for practicing any one of the methods of the invention.

The details of one or more aspects of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The following drawings are illustrative of aspects of the invention and are not meant to limit the scope of the invention as encompassed by the claims.

FIG. 1 illustrates data from an exemplary AD to ADD conversion assay: FIG. 1A illustrates data from a random Tn5 mutant; FIG. 1B illustrates data from a ksdA Tn5 mutant, showing the absence of AD to ADD conversion; as discussed in detail in Example 1, below.

FIG. 2 illustrates data from an exemplary cholesterol conversion assay (X2 only):

FIG. 2A uses the random Tn5 mutant, and FIG. 2B uses the cxgB Tn5 mutant 1, showing absence of Compound X2 production; as discussed in detail in Example 1, below.

FIG. 3 illustrates data from an exemplary cholesterol conversion assay (X1 and X2), showing absence of compounds X1 and X2 production: FIG. 3A uses the random Tn5 mutant, FIG. 3B uses the cxgA Tn5 mutant 2, and FIG. 3C uses the cxgA Tn5 mutant 3; as discussed in detail in Example 1, below.

FIG. 4 graphically illustrates data showing a time course for conversion of cholesterol to AD and ADD by wild-type and ΔksdA/ΔcxgB mutant; as discussed in detail in Example 1, below.

FIG. 5 graphically illustrates data showing a time course for conversion of cholesterol to Compound X1 and X2 by wild-type and ΔksdA/ΔcxgB mutant; as discussed in detail in Example 1, below.

FIG. 6 is a schematic illustration of an exemplary chromosomal site of insertion and gene organization around the 3-ketosteroid-Δ1-dehydrogenase mutation abolishing AD to ADD conversion; as discussed in detail in Example 1, below.

FIG. 7 is a schematic illustration of exemplary chromosomal sites of insertions and organization of the “cxg genes”, i.e., the cxgA, cxgB, cxgC, or cxgD genes; as discussed in detail in Example 1, below.

Like reference symbols in the various drawings indicate like elements.

### DETAILED DESCRIPTION OF THE INVENTION

The invention provides methods for producing androstenedione (AD, or 4-androstene-3,17-dione) of “improved” purity (e.g., a more pure, or relatively pure, or substantially pure, AD) and for modulating AD production, for example by deletion or inactivation of a nucleic acid, e.g., a gene, encoding ksdA, cxgA, cxgB, cxgC, or cxgD (SEQ ID NO:1, SEQ ID NO:9, SEQ ID NO:17, SEQ ID NO:24 and SEQ ID NO:31, respectively). The invention also provides nucleic acids that encode proteins for producing 1,4-androstadiene-3,17-dione (ADD) and related pathway compounds, including 20-(hydroxymethyl)pregna-4-en-3-one and 20-(hydroxymethyl)pregna-1,4-dien-3-one. In alternative embodiments, these proteins comprise genuses based the exemplary amino acid sequences SEQ ID NO:2, SEQ ID NO:10 (and SEQ ID NO:11), SEQ ID NO:18, SEQ ID NO:25, SEQ ID NO:32.

The invention provides isolated, recombinant and isolated nucleic acids having a sequence comprising the coding sequence of the polypeptide KsdA, including the gene sequence ksdA (SEQ ID NO:1), and an amino acid sequence encoded by ksdA (SEQ ID NO:2), and enzymatically active fragments thereof, wherein the enzyme activity comprises a 3-ketosteroid-Δ1-dehydrogenase activity. In one embodiment, the invention also provides functionally active ksdA nucleic acid and KsdA polypeptide variants (e.g., as isolated, recombinant and isolated nucleic acids or polypeptides, respectively) comprising a sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:1 or SEQ ID NO:2, respectively, wherein the functional activity, or the enzyme activity (including activity for the enzymatically active fragment), comprises a 3-ketosteroid-Δ1-dehydrogenase activity. In one aspect, the sequence identities are determined by analysis with a sequence comparison algorithm or by a visual inspection.

In one embodiment, the invention provides isolated, recombinant and isolated polypeptides comprising an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to the amino acid sequences SEQ ID NO:2, SEQ ID NO:3, SEQ ID NO:4, SEQ ID NO:5, SEQ ID NO:6, SEQ ID NO:7 and/or SEQ ID NO:8, or the consensus sequence between two or more of the amino acid sequences SEQ ID NO:2, SEQ ID NO:3, SEQ ID NO:4, SEQ ID NO:5, or among all the amino acid sequences SEQ ID NO:2, SEQ ID NO:3, SEQ ID NO:4, SEQ ID NO:5, SEQ ID NO:6, SEQ ID NO:7 and/or SEQ ID NO:8; wherein the enzyme activity of the polypeptide comprises a 3-ketosteroid-Δ1-dehydrogenase activity. In one aspect, the sequence identities are determined by analysis with a sequence comparison algorithm or by a visual inspection. In one aspect, the invention encompasses and provides nucleic acids encoding any polypeptide of the invention, including these consensus sequence polypeptides.

In one embodiment, the invention provides isolated, recombinant and isolated nucleic acids comprising a nucleic acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to the gene sequences of cxgA, cxgB, cxgC, cxgD, as set forth respectively in SEQ ID NO:9, SEQ ID NO:17, SEQ ID NO:24 and SEQ ID NO:31; and CxgA, CxgB, CxgC, CxgD amino acid sequences comprising the sequences as set forth respectively in SEQ ID NO:10 (and SEQ ID NO:11), SEQ ID NO:18, SEQ ID NO:25 and SEQ ID NO:32, as well as their enzymatically active or DNA-binding fragments; wherein the enzyme or protein activity (including an enzymatically active fragment) for CxgA, CxgB, CxgC, CxgD comprises an acetyl CoA-acetyltransferase/thiolase activity (CxgA), a DNA-binding protein activity (CxgB), an acyl-CoA dehydrogenase/FadE protein activity (CxgC), and TetR-like regulatory protein/KstR activity (CxgD), respectively.

In one embodiment, the invention provides isolated, recombinant and isolated polypeptides comprising a polypeptide sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to the amino acid sequence of

(1) the respective consensus sequence between the amino acid sequences SEQ ID NO:10, SEQ ID NO:11 and SEQ ID NO:12, or a consensus sequence among two or more or all of the amino acid sequences SEQ ID NO:10, SEQ ID NO:11, SEQ ID NO:12, SEQ ID NO:13, SEQ ID NO:14, SEQ ID NO:15 and SEQ ID NO:16; wherein polypeptide is CxgA enzyme activity, e.g., an acetyl CoA-acetyltransferase/thiolase activity:

(2) the respective consensus sequence between the amino acid sequences SEQ ID NO:18, SEQ ID NO:19 and SEQ ID NO:20, or a consensus sequence among two or more or all of the amino acid sequences SEQ ID NO:18, SEQ ID NO:19, SEQ ID NO:20, SEQ ID NO:21, SEQ ID NO:22 and SEQ ID NO:23; wherein polypeptide has a CxgB protein activity, e.g., a DNA-binding activity:

(3) the respective consensus sequence between the amino acid sequences SEQ ID NO:25, SEQ ID NO:26 and SEQ ID NO:27, or a consensus sequence among two or more or all of the amino acid sequences SEQ ID NO:25, SEQ ID NO:26, SEQ ID NO:27, SEQ ID NO:28, SEQ ID NO:29 and SEQ ID NO:30; wherein polypeptide has a CxgC enzyme activity, e.g., an acyl-CoA dehydrogenase/FadE enzyme activity; and/or

(4) the respective consensus sequence between the amino acid sequences SEQ ID NO:32, SEQ ID NO:33 and SEQ ID NO:34, or a consensus sequence among two or more or all of the amino acid sequences SEQ ID NO:32, SEQ ID NO:33, SEQ ID NO:34, SEQ ID NO:35, and SEQ ID NO:36; wherein polypeptide has a CxgD enzyme activity, e.g., a TetR-like regulatory protein/KstR activity.

In one aspect, the invention encompasses and provides nucleic acids encoding any polypeptide of the invention, including these consensus sequence polypeptides.

The invention further provides methods for modulating the production of ADD and related pathway compounds, including 20-(hydroxymethyl)pregna-4-en-3-one and 20-(hydroxymethyl)pregna-1,4-dien-3-one, for example by over- or underexpressing any one of, or several of, or all of ksdA, cxgA, cxgB, cxgC and/or cxgD (SEQ ID NO:1, SEQ ID NO:9, SEQ ID NO:17, SEQ ID NO:24 and SEQ ID NO:31, respectively).

The invention provides nucleic acids, e.g., as genes and/or enzyme coding sequences, responsible for the production of androstadienedione and compounds 1,4-androstadiene-3,17-dione (ADD), 20-(hydroxymethyl)pregna-4-en-3-one (referred to here as compound X1) and 20-(hydroxymethyl)pregna-1,4-dien-3-one (referred to here as compound X2). In one embodiment, the invention provides methods for the deletion and/or inactivation (e.g., by base mutation, addition (e.g., insertions), deletion) of one or all of these nucleic acids, e.g., as genes and/or enzyme coding sequences, to generate a novel host for the economical production of androstenedione, X1 and/or X2, and host cells resulting from these methods, e.g., host cells modified such that their genes and/or coding sequences (e.g., messages, mRNA) for androstenedione, X1 and/or X2 are deleted or inactivated (which would include removal, modification or deletion of substantially most active forms). In one aspect, the modified host cell of the invention is a bacterial cell, e.g., a Mycobacterium strain, such as a Mycobacterium strain designated B3683 or B3805.

### Nucleic Acids, Expression Vehicles and Systems and Host Cells

In one aspect, the invention provides isolated, recombinant and synthetic nucleic acids having a sequence identity to an exemplary sequence of the invention, e.g., SEQ ID NO:1, SEQ ID NO:9, SEQ ID NO:17, SEQ ID NO:24 and SEQ ID NO:31, etc.; nucleic acids encoding polypeptides of the invention, e.g., exemplary polypeptides of the invention, e.g., SEQ ID NO:2, SEQ ID NO:10 (and SEQ ID NO:11), SEQ ID NO:18, SEQ ID NO:25, SEQ ID NO:32, etc.) including expression cassettes such as expression vectors, encoding the polypeptides of the invention. In one embodiment, the invention provides methods for making cells that underexpress (as compared to a wild type or unmanipulated cell) or do not express any one, or several of, or all ksdA-, cxgA-, cxgB-, cxgC- and/or cxgD (SEQ ID NO:1, SEQ ID NO:9, SEQ ID NO:17, SEQ ID NO:24 and SEQ ID NO:31, respectively) polypeptide-encoding nucleic acids in a cell.

The nucleic acids of the invention can be made, isolated and/or manipulated by, e.g., cloning and expression of cDNA libraries, amplification of message or genomic DNA by PCR, and the like. For example, exemplary sequences of the invention were initially derived from environmental sources. Regarding the term “derived” for purposes of the specification and claims, in some aspects, a substance is “derived” from an organism or source if any one or more of the following are true: 1) the substance is present in the organism/source; 2) the substance is removed from the native host; or, 3) the substance is removed from the native host and is evolved, for example, by mutagenesis.

The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense (complementary) strand, to peptide nucleic acid (PNA), or to any DNA-like or RNA-like material, natural or synthetic in origin. The phrases “nucleic acid” or “nucleic acid sequence” includes oligonucleotide, nucleotide, polynucleotide, or to a fragment of any of these, to DNA or RNA (e.g., mRNA, rRNA, tRNA, iRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., e.g., double stranded iRNAs, e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. “Oligonucleotide” includes either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated.

A “coding sequence of” or a “nucleotide sequence encoding” a particular polypeptide or protein, is a nucleic acid sequence which is transcribed and translated into a polypeptide or protein when placed under the control of appropriate regulatory sequences. The term “gene” means the segment of DNA involved in producing a polypeptide chain; it includes regions preceding and following the coding region (leader and trailer) as well as, where applicable, intervening sequences (introns) between individual coding segments (exons). “Operably linked” as used herein refers to a functional relationship between two or more nucleic acid (e.g., DNA) segments. Typically, it refers to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance

In practicing the methods of the invention, homologous genes can be modified by manipulating a template nucleic acid, as described herein. The invention can be practiced in conjunction with any method or protocol or device known in the art, which are well described in the scientific and patent literature.

In alternative embodiments, nucleic acids used to practice this invention can comprise DNA, including cDNA, genomic DNA and synthetic DNA. The DNA may be double-stranded or single-stranded and if single stranded may be the coding strand or non-coding (anti-sense) strand. Alternatively, nucleic acids used to practice this invention can comprise RNA, e.g., mRNA, RNAi and the like.

Nucleic acids of this invention can be used to prepare polypeptides of the invention, which include enzymatically active fragments thereof. In alternative embodiments, nucleic acids that encode polypeptides of the invention include: polypeptide coding sequences of a nucleic acid of the invention, and optionally additional coding sequences, such as leader sequences or proprotein sequences and non-coding sequences, such as introns or non-coding sequences 5′ and/or 3′ of the coding sequence. Thus, as used herein, the term “polynucleotide encoding a polypeptide” encompasses both polynucleotides comprising protein coding sequences and polynucleotide sequences comprising additional coding and/or non-coding sequences, e.g., transcriptional or translational regulatory sequences.

In alternative embodiments, nucleic acid sequences of the invention can be mutagenized using conventional techniques, such as site directed mutagenesis, or other techniques familiar to those skilled in the art, to introduce silent changes into the polynucleotides of the invention. As used herein, “silent changes” include, for example, changes which do not alter the amino acid sequence encoded by the polynucleotide. Such changes may be desirable in order to increase the level of the polypeptide produced by host cells containing a vector encoding the polypeptide by introducing codons or codon pairs which occur frequently in the host organism.

The invention also encompasses polynucleotides having nucleotide changes which result in amino acid substitutions, additions, deletions, fusions and truncations in the polypeptides of the invention; and methods for making such changes to ksdA-, cxgA-, cxgB-, cxgC- and/or cxgD-encoding nucleic acids (e.g., genes) (SEQ ID NO:1, SEQ ID NO:9, SEQ ID NO:17, SEQ ID NO:24 and SEQ ID NO:31, respectively) to generate a cell that over- or under-expresses one several or all of these nucleic acids. Such nucleotide changes may be introduced into the nucleic acid, including introducing such changes directly into a cell, using techniques such as site directed mutagenesis, random chemical or radiation mutagenesis, exonuclease III deletion, insertional transposons and other recombinant mutation-inducing techniques. Alternatively, such nucleotide changes may be made using naturally occurring allelic variants.

The term “variant” refers to polynucleotides or polypeptides of the invention modified at one or more base pairs, codons, introns, exons, or amino acid residues (respectively) yet still retain the biological activity. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof.

General Techniques

The nucleic acids used to practice this invention, whether RNA, siRNA, miRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides (e.g., the exemplary KsdA-, CxgA-, CxgB-, CxgC- and/or CxgD enzymes) (SEQ ID NO:2, SEQ ID NO:10 (and SEQ ID NO:11), SEQ ID NO:18, SEQ ID NO:25, SEQ ID NO:32, respectively) generated from these nucleic acids can be individually isolated or cloned and tested for a desired activity.

Any recombinant expression system can be used, including bacterial (e.g., Mycobacterial), mammalian, fungal, yeast, insect or plant cell expression systems. “Recombinant” polypeptides or proteins refer to polypeptides or proteins produced by recombinant DNA techniques; i.e., produced from cells transformed by an exogenous DNA construct encoding the desired polypeptide or protein. “Synthetic” polypeptides or protein are those prepared by chemical synthesis. Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Commercially available laboratory kits can be utilized as described in H. M. Geysen et al, Proc. Natl. Acad. Sci., USA, 81:3998 (1984), e.g., synthesizing peptides upon the tips of a multitude of “rods” or “pins” all of which are connected to a single plate. In one embodiment, the term “recombinant” means that the nucleic acid is adjacent to a “backbone” nucleic acid to which it is not adjacent in its natural environment.

In one embodiment, nucleic acids used to practice this invention are synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

In one embodiment, obtaining and manipulating nucleic acids used to practice the invention include cloning from genomic samples, and, if desired, screen and re-clone inserts isolated or amplified from, e.g., genomic clones or cDNA clones. Sources of nucleic acid used to practice the invention include genomic or cDNA libraries contained in, e.g., mammalian artificial chromosomes (MACS), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one embodiment, the term “isolated” as used herein refers to any substance removed from its native host; the substance need not be purified. For example “isolated nucleic acid” refers to a naturally-occurring nucleic acid that is not immediately contiguous with both of the sequences with which it is immediately contiguous (one on the 5′ end and one on the 3′ end) in the naturally-occurring genome of the organism from which it is derived. In one embodiment, an isolated nucleic acid can be, without limitation, a recombinant DNA molecule of any length, provided one of the nucleic acid sequences normally found immediately flanking that recombinant DNA molecule in a naturally-occurring genome is removed or absent. In one embodiment, an isolated nucleic acid includes a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In one embodiment, an isolated nucleic acid can include a recombinant DNA molecule that is part of a hybrid or fusion nucleic acid sequence.

In one aspect, the term “isolated” means that the material (e.g., a protein or nucleic acid of the invention) is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

In one aspect, the term “isolated” as used with reference to nucleic acids also can include any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

In one embodiment, the terms “purified” or “relative purity” as used herein does not require absolute purity, but rather “purified” and “relative purity” are intended as a relative term. Thus, for example, a purified or relatively purified desired product such as an androstenedione (AD, or a polypeptide or nucleic acid, can be one in which the desired product (e.g., AD), polypeptide or nucleic acid is at a higher concentration than the desired product, polypeptide or nucleic acid would be (or would have been made) in its natural environment within an organism (e.g., in an unmanipulated cell) or at a higher concentration than in the environment from which it was removed or found (generated) in an unmanipulated cell.

In one embodiment, the terms “purified” or “relative purity” encompass the term “enriched”; and in one aspect, to be “enriched” or having “relative greater purity” a nucleic acid, polypeptide or desired product, e.g., androstenedione (AD, or (4-androstene-3,17-dione) has at least about 1.0%, 2.0%, 3.0%, 4.0%, 5.0%, 10.0%, 10.5%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more fewer (lesser) impurities, including for example fewer (lesser) impurities in the AD synthesis process, e.g. where the fewer impurities comprise fewer androstadienedione (ADD), 20-(hydroxymethyl)pregna-4-en-3-one, 20-(hydroxymethyl)pregna-1,4-dien-3-one, and related compounds considered “impurities” or “contaminants” in the cell-based AD synthesis process.

Transcriptional and Translational Control Sequences

The invention provides nucleic acid (e.g., DNA) sequences of the invention, and inhibitory sequences (e.g., to the exemplary ksdA, cxgA, cxgB, cxgC and/or cxgD) (SEQ ID NO:1, SEQ ID NO:9, SEQ ID NO:17, SEQ ID NO:24 and SEQ ID NO:31, respectively), operatively linked to expression (e.g., transcriptional or translational) control sequence(s), e.g., promoters or enhancers, to direct or modulate nucleic acid (e.g., RNA, message) synthesis/expression. The expression control sequence can be in an expression vehicle, e.g., a vector. Exemplary bacterial promoters include lacI, lacZ, T3, T7, gpt, lambda P_{R}, P_{L} and trp. Exemplary eukaryotic promoters include CMV immediate early, HSV thymidine kinase, early and late SV40, LTRs from retrovirus, and mouse metallothionein I.

In alternative embodiments, promoters suitable for use in practicing this invention, e.g., for expressing a polypeptide in cell, e.g., a bacteria, include the E. coli lac or trp promoters, the lacI promoter, the lacZ promoter, the T3 promoter, the T7 promoter, the gpt promoter, the lambda P_{R} promoter, the lambda P_{L} promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. In alternative embodiments, any promoter or enhancer known to control expression of a gene or transcript in a prokaryotic or a eukaryotic cell, or a virus, can be used.

In alternative embodiments, promoters suitable for use in practicing this invention include all sequences capable of driving transcription of a coding sequence in a cell, e.g., a bacterial, yeast, fungal or plant cell and the like. Thus, promoters used in the constructs of the invention can include cis-acting transcriptional control elements and regulatory sequences that are involved in regulating or modulating the timing and/or rate of transcription of a gene. In alternative embodiments, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. In alternative embodiments, cis-acting sequences can interact with proteins or other biomolecules to carry out (turn on/off, regulate, modulate, etc.) transcription. In alternative embodiments, “constitutive” promoters that drive expression continuously under most environmental conditions and states of development or cell differentiation are used. In alternative embodiments, “inducible” or “regulatable” promoters that direct expression of a nucleic acid under the influence of environmental conditions or developmental conditions are used. Examples of environmental conditions that may affect transcription by inducible promoters include anaerobic conditions, elevated temperature, drought, or the presence of light. In alternative embodiments, “tissue-specific” promoters that are only active in particular cells or tissues or organs, e.g., in certain bacteria, tissues or organs, plants or animals, are used. Tissue-specific regulation may be achieved by certain intrinsic factors which ensure that genes encoding proteins specific to a given tissue are expressed.

Expression Cassettes, Vectors and Cloning Vehicles

The invention provides expression cassettes and vectors and cloning vehicles comprising nucleic acids of the invention, e.g., sequences encoding the KsdA, CxgA, CxgB, CxgC and/or CxgD (SEQ ID NO:2, SEQ ID NO:10 (and SEQ ID NO:11), SEQ ID NO:18, SEQ ID NO:25, SEQ ID NO:32, respectively) enzyme genuses of the invention. In alternative embodiments, expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest, such as a member of the family Mycobacteriaceae, Nocardiaceae, Bacillaceae, Trichocomaceae or Saccharomycetaceae. Vectors of the invention can include chromosomal, non-chromosomal and synthetic DNA sequences. In alternative embodiments, any suitable vector known to those of skill in the art or commercially available can be used. Exemplary vectors are include: bacterial: pQE vectors (Qiagen), pBLUESCRIPT™ plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention. “Plasmids” can be commercially available, publicly available on an unrestricted basis, or can be constructed from available plasmids in accord with published procedures. Equivalent plasmids to those described herein are known in the art and will be apparent to the ordinarily skilled artisan.

In alternative embodiments, “expression cassettes” comprising a nucleotide sequence which is capable of affecting expression of a structural gene (i.e., KsdA-, CxgA-, CxgB-, CxgC- and/or CxgD-encoding nucleic acid) in a host compatible with such sequences are used. In alternative embodiments, expression cassettes include at least a promoter operably linked with the polypeptide coding sequence; and, optionally, with other sequences, e.g., transcription termination signals. In alternative embodiments, additional factors necessary or helpful in effecting expression may also be used, e.g., enhancers, alpha-factors. In alternative embodiments, expression cassettes also include plasmids, expression vectors, recombinant viruses, any form of recombinant “naked DNA” vector, and the like.

In alternative embodiments, “vectors” of the invention comprise a nucleic acid which can infect, transfect, transiently or permanently transduce a cell. In alternative embodiments, a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In alternative embodiments, a recombinant microorganism or cell culture, e.g., as described herein as hosting an “expression vector”, can include both extra-chromosomal circular and linear DNA and/or DNA that has been incorporated into a host chromosome(s). In alternative embodiments, where a vector is being maintained by a host cell, the vector may either be stably replicated by the cells during mitosis as an autonomous structure, or is incorporated within the host's genome.

In alternative embodiments, the expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

In alternative embodiments, vectors for expressing a polypeptide or nucleic acid used to practice this invention also can contain enhancers to increase expression levels. Enhancers are cis-acting elements of DNA that can be from about 10 to about 300 bp in length. They can act on a promoter to increase its transcription. Exemplary enhancers include the SV40 enhancer on the late side of the replication origin by 100 to 270, the cytomegalovirus early promoter enhancer, the polyoma enhancer on the late side of the replication origin, and the adenovirus enhancers.

In alternative embodiments, a nucleic acid sequence is inserted into a vector by a variety of procedures; e.g., a sequence can be ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

In alternative embodiments, bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBLUESCRIPT II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

The nucleic acids of the invention can be expressed in expression cassettes, vectors or viruses and transiently or stably expressed in any cell, including bacteria, plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a cell, e.g., a bacterial cell, a plant cell or a seed. For example, the marker may encode biocide resistance, particularly antibiotic resistance, such as resistance to kanamycin, G418, bleomycin, hygromycin, or herbicide resistance, such as resistance to chlorosulfuron or Basta.

In alternative embodiments, expression vectors capable of expressing nucleic acids and proteins in plants that are well known in the art can be used and include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in plant, mammalian or insect cells for expression and in a prokaryotic host, e.g., bacterial cell, for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

The DNA sequence in the expression vector is operatively linked to an appropriate expression control sequence(s) (promoter) to direct RNA synthesis. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using chloramphenicol transferase (CAT) vectors or other vectors with selectable markers. In addition, the expression vectors in one aspect contain one or more selectable marker genes to provide a phenotypic trait for selection of transformed host cells such as dihydrofolate reductase or neomycin resistance for eukaryotic cell culture, or such as tetracycline or ampicillin resistance in E. coli.

In addition, the expression vectors typically contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in Mycobacteriaceae or E. coli and/or a S. cerevisiae TRP1 gene.

Host Cells and Transformed Cells

The invention also provides a transformed cell comprising a nucleic acid sequence of the invention, e.g., KsdA-, CxgA-, CxgB-, CxgC- and/or CxgD-encoding nucleic acids of the invention, or a vector of the invention. The invention also provides cells for producing androstenedione (AD), androstadienedione (ADD), 20-(hydroxymethyl)pregna-4-en-3-one and/or 20-(hydroxymethyl)pregna-1,4-dien-3-one, where in alternative embodiments the cells comprise the over- or underexpressing of any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell, or deletion of the expression of any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell.

In alternative embodiments any host cell can be used, e.g., any of the host cells familiar to those skilled in the art, including prokaryotic cells, eukaryotic cells, such as bacterial cells, fungal cells, yeast cells, mammalian cells, insect cells, or plant cells. Exemplary bacterial cells include any member of the genus Actinobacteria, or any member of the family Mycobacteriaceae, any species of Streptomyces, Staphylococcus, Pseudomonas or Bacillus, including E. coli, Bacillus subtilis, Pseudomonas fluorescens, Bacillus cereus, or Salmonella typhimurium. Exemplary fungal cells include any species of Aspergillus. Exemplary yeast cells include any species of Pichia, Saccharomyces, Schizosaccharomyces, or Schwanniomyces, including Pichia pastoris, Saccharomyces cerevisiae, or Schizosaccharomyces pombe. Exemplary insect cells include any species of Spodoptera or Drosophila, including Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477; U.S. Pat. No. 5,750,870.

In alternative embodiments vectors are introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets can be used.

In alternative embodiments the engineered host cells are cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

In alternative embodiments cells are harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Host cells containing the polynucleotides of interest, e.g., nucleic acids of the invention, can be cultured in conventional nutrient media modified as appropriate for activating promoters, selecting transformants or amplifying genes. The culture conditions, such as temperature, pH and the like, are those previously used with the host cell selected for expression and will be apparent to the ordinarily skilled artisan. The clones which are identified as having the specified enzyme activity may then be sequenced to identify the polynucleotide sequence encoding an enzyme having the enhanced activity.

The nucleic acids of the invention can be expressed, or overexpressed, in any in vitro or in vivo expression system. Any cell culture systems can be employed to express, or over-express, recombinant protein, including bacterial, insect, yeast, fungal or mammalian cultures. Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the polypeptides of the invention.

Amplification of Nucleic Acids

In practicing the invention, nucleic acids of the invention, e.g., the exemplary KsdA, CxgA, CxgB, CxgC and/or CxgD-encoding nucleic acids (including e.g. SEQ ID NO:1, SEQ ID NO:9, SEQ ID NO:17, SEQ ID NO:24 and SEQ ID NO:31, respectively), can be reproduced by amplification. Amplification can also be used to clone or modify the nucleic acids of the invention. Thus, the invention provides amplification primer sequence pairs for amplifying nucleic acids of the invention, including exemplary sequences of the invention. One of skill in the art can design amplification primer sequence pairs for any part of or the full length of these sequences.

In one aspect, the invention provides a nucleic acid amplified by a primer pair of the invention, e.g., a primer pair as set forth by about the first (the 5′) 11, 12, 13, 14, 15, 16, 17, 18, 19, 20, 21, 22, 23, 24, or 25 or more residues of a nucleic acid of the invention, and about the first (the 5′) 11, 12, 13, 14, 15, 16, 17, 18, 19, 20, 21, 22, 23, 24, or 25 or more residues of the complementary strand.

The invention provides an amplification primer sequence pair for amplifying a nucleic acid encoding a polypeptide, e.g., KsdA, CxgA, CxgB, CxgC and/or CxgD, wherein the primer pair is capable of amplifying a nucleic acid comprising a sequence of the invention, or fragments or subsequences thereof. One or each member of the amplification primer sequence pair can comprise an oligonucleotide comprising at least about 10 to 50 or more consecutive bases of the sequence, or about 11, 12, 13, 14, 15, 16, 17, 18, 19, 20, 21, 22, 23, 24, or 25 or more consecutive bases of the sequence. The invention provides amplification primer pairs, wherein the primer pair comprises a first member having a sequence as set forth by about the first (the 5′) 11, 12, 13, 14, 15, 16, 17, 18, 19, 20, 21, 22, 23, 24, or 25 or more residues of a nucleic acid of the invention, and a second member having a sequence as set forth by about the first (the 5′) 12, 13, 14, 15, 16, 17, 18, 19, 20, 21, 22, 23, 24, or 25 or more residues of the complementary strand of the first member.

The invention provides KsdA, CxgA, CxgB, CxgC and/or CxgD (SEQ ID NO:2, SEQ ID NO:10 (and SEQ ID NO:11), SEQ ID NO:18, SEQ ID NO:25, SEQ ID NO:32, respectively) enzymes generated by amplification, e.g., polymerase chain reaction (PCR), using an amplification primer pair of the invention. The invention provides methods of making KsdA, CxgA, CxgB, CxgC and/or CxgD enzymes by amplification, e.g., polymerase chain reaction (PCR), using an amplification primer pair of the invention. In one aspect, the amplification primer pair amplifies a nucleic acid from a library, e.g., a gene library, such as an environmental library.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

### Determining the Degree of Sequence Identity

The invention provides nucleic acids comprising sequences having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary nucleic acid or polypeptide of the invention, including enzymatically active fragments thereof), and nucleic acids encoding them (including both strands, i.e., sense and nonsense, coding or noncoding). The extent of sequence identity (homology) may be determined using any computer program and associated parameters, including those described herein, such as BLAST 2.2.2. or FASTA version 3.0t78, with the default parameters.

Nucleic acid sequences of the invention can comprise at least 10, 15, 20, 25, 30, 35, 40, 50, 75, 100, 150, 200, 300, 400, or 500 or more consecutive nucleotides of an exemplary sequence of the invention and sequences substantially identical thereto.

Sequence identity (homology) may be determined using any of the computer programs and parameters described herein, including FASTA version 3.0t78 with the default parameters. In alternative aspects, homologous sequences also include RNA sequences in which uridines replace the thymines in the nucleic acid sequences of the invention. The homologous sequences may be obtained using any of the procedures described herein or may result from the correction of a sequencing error. It will be appreciated that the nucleic acid sequences of the invention can be represented in the traditional single character format (See the inside back cover of Stryer, Lubert. Biochemistry, 3rd Ed., W. H Freeman & Co., New York.) or in any other format which records the identity of the nucleotides in a sequence.

As used herein, the terms “computer,” “computer program” and “processor” are used in their broadest general contexts and incorporate all such devices, as described in detail, below. A “coding sequence of” or a “sequence encodes” a particular polypeptide or protein, is a nucleic acid sequence which is transcribed and translated into a polypeptide or protein when placed under the control of appropriate regulatory sequences.

In alternative embodiments, any sequence comparison program with any computer can be used. In alternative embodiments, protein and/or nucleic acid sequence identities (homologies) are evaluated using any of the variety of sequence comparison algorithms and programs and computers known in the art; e.g., such algorithms and programs include TBLASTN, BLASTP, FASTA, TFASTA and CLUSTALW (see, e.g., Pearson and Lipman, Proc. Natl. Acad. Sci. USA 85(8):2444-2448, 1988; Altschul et al., J. Mol. Biol. 215(3):403-410, 1990; Thompson Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

In alternative embodiments, homology or identity is measured using sequence analysis software embedded in a computer, e.g., using the Sequence Analysis Software Package of the Genetics Computer Group, University of Wisconsin Biotechnology Center, 1710 University Avenue, Madison, Wis. 53705. In alternative embodiments, software matches similar sequences by assigning degrees of sequence identities (homology) to various deletions, substitutions and other modifications. The terms “homology” and “sequence identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

In alternative embodiments, for sequence comparison, one sequence acts as a reference sequence, to which test sequences are compared. When using a sequence comparison algorithm, test and reference sequences can be entered into a computer, subsequence coordinates are designated, if necessary and sequence algorithm program parameters are designated. Default program parameters can be used, or alternative parameters can be designated. In alternative embodiments, the sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. In alternative embodiments, optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol 48:443, 1970, by the search for similarity method of Lipman (1988) Proc. Nat'l. Acad. Sci. USA 85:2444, by computerized implementations of these algorithms (GAP™, BESTFIT™, FASTA and TFASTA in the Wisconsin Genetics Software Package, Genetics Computer Group, 575 Science Dr., Madison, Wis.), or by manual alignment and visual inspection.

In alternative embodiments, algorithms for determining homology or identity include, for example, in addition to a BLAST program (Basic Local Alignment Search Tool at the National Center for Biological Information), ALIGN™, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN™, Las Vegas algorithm, FNAT (Forced Nucleotide Alignment Tool), FRAMEALIGN™, FRAMESEARCH™, DYNAMIC™, FILTER™, FSAP™ (Fristensky Sequence Analysis Package), GAP (Global Alignment Program), GENAL™, GIBBS™, GENQUEST™, ISSC™ (Sensitive Sequence Comparison), LALIGN™ (Local Sequence Alignment), LCP™ (Local Content Program), MACAW™ (Multiple Alignment Construction & Analysis Workbench), MAP (Multiple Alignment Program), MBLKP™, MBLKN™, PIMA™ (Pattern-Induced Multi-sequence Alignment), SAGA™ (Sequence Alignment by Genetic Algorithm) and WHAT-IF™. Such alignment programs can also be used to screen genome databases to identify polynucleotide sequences having substantially identical sequences.

In alternative embodiments, BLAST and BLAST 2.0 algorithms are used, e.g. described in Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

The BLAST algorithm also performs a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more in one aspect less than about 0.01 and most in one aspect less than about 0.001.

In one aspect, protein and nucleic acid sequence homologies are evaluated using the Basic Local Alignment Search Tool (“BLAST”) In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

In alternative embodiments, BLAST programs are used to identify homologous sequences by identifying similar segments, which are referred to herein as “high-scoring segment pairs,” between a query amino or nucleic acid sequence and a test sequence which is in one aspect obtained from a protein or nucleic acid sequence database. High-scoring segment pairs are in one aspect identified (i.e., aligned) by means of a scoring matrix, many of which are known in the art. In one aspect, the scoring matrix used is the BLOSUM62 matrix (Gonnet (1992) Science 256:1443-1445; Henikoff and Henikoff (1993) Proteins 17:49-61). Less in one aspect, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST programs are accessible through the U.S. National Library of Medicine.

The parameters used with the above algorithms may be adapted depending on the sequence length and degree of homology studied. In some aspects, the parameters may be the default parameters used by the algorithms in the absence of instructions from the user.

### Computer Systems and Computer Program Products

In one embodiment, the invention provides computer systems comprising a processor and a data storage or a machine readable memory device wherein said data storage device has stored thereon a polypeptide sequence or a nucleic acid sequence, wherein the polypeptide sequence comprises the polypeptide (amino acid) sequence of the invention or a polypeptide encoded by the nucleic acid (polynucleotide) sequence of the invention.

To determine and identify sequence identities, structural homologies, motifs and the like in silico, a nucleic acid or polypeptide sequence of the invention can be stored, recorded, and manipulated on any medium which can be read and accessed by a computer. In alternative embodiments the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Homology (sequence identity) may be determined using any of the computer programs and parameters described herein operatively saved on a computer. A nucleic acid or polypeptide sequence of the invention can be stored, recorded and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid sequences of the invention, one or more of the polypeptide sequences of the invention. Another aspect of the invention is a computer readable medium having recorded thereon at least 2, 5, 10, 15, or 20 or more nucleic acid or polypeptide sequences of the invention.

Another aspect of the invention is a computer readable medium having recorded thereon one or more of the nucleic acid sequences of the invention. Another aspect of the invention is a computer readable medium having recorded thereon one or more of the polypeptide sequences of the invention. Another aspect of the invention is a computer readable medium having recorded thereon at least 2, 5, 10, 15, or 20 or more of the nucleic acid or polypeptide sequences as set forth above.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

In alternative embodiments, programs and databases which are operatively saved and used with computers include e.g., MACPATTERN™ (EMBL), DISCOVERYBASE™ (Molecular Applications Group), GENEMINE™ (Molecular Applications Group), LOOK™ (Molecular Applications Group), MACLOOK™ (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB™ (Brutlag et al. Comp. App. Biosci. 6:237-245, 1990), CATALYST™ (Molecular Simulations Inc.), CATALYST™/SHAPE™ (Molecular Simulations Inc.), CERIUS².DBACCESS™ (Molecular Simulations Inc.), HYPOGEN™ (Molecular Simulations Inc.), INSIGHT II™, (Molecular Simulations Inc.), DISCOVER™ (Molecular Simulations Inc.), CHARMm™ (Molecular Simulations Inc.), FELIX™ (Molecular Simulations Inc.), DELPHI™ (Molecular Simulations Inc.), QUANTEMM™, (Molecular Simulations Inc.), HOMOLOGY™ (Molecular Simulations Inc.), MODELER™ (Molecular Simulations Inc.), ISIS™ (Molecular Simulations Inc.), QUANTA™/Protein Design (Molecular Simulations Inc.), WEBLAB™ (Molecular Simulations Inc.), WEBLAB DIVERSITY EXPLORER™ (Molecular Simulations Inc.), GENE EXPLORER™ (Molecular Simulations Inc.), SEQFOLD™ (Molecular Simulations Inc.), the MDL Available Chemicals Directory database, the MDL Drug Data Report data base, the Comprehensive Medicinal Chemistry database, Derwents' World Drug Index database, the BioByteMasterFile database, the Genbank database and the Genseqn database.

Motifs which may be detected using the above programs include sequences encoding leucine zippers, helix-turn-helix motifs, glycosylation sites, ubiquitination sites, alpha helices and beta sheets, signal sequences encoding signal peptides which direct the secretion of the encoded proteins, sequences implicated in transcription regulation such as homeoboxes, acidic stretches, enzymatic active sites, substrate binding sites and enzymatic cleavage sites.

### Hybridization of Nucleic Acids

The invention provides isolated, synthetic or recombinant nucleic acids that hybridize under stringent conditions to a sequence of the invention, including any exemplary sequence of the invention. The stringent conditions can be highly stringent conditions, medium stringent conditions and/or low stringent conditions, including the high and reduced stringency conditions described herein. In one aspect, it is the stringency of the wash conditions that set forth the conditions which determine whether a nucleic acid is within the scope of the invention, as discussed below.

In one embodiment, “hybridization” refers to the process by which a nucleic acid strand joins with a complementary strand through base pairing; hybridization reactions can be sensitive and selective so that a particular sequence of interest can be identified even in samples in which it is present at low concentrations. In alternative embodiments, stringent conditions are defined by the concentrations of salt or formamide in the prehybridization and hybridization solutions, or by the hybridization temperature and are well known in the art. In particular, stringency can be increased by reducing the concentration of salt, increasing the concentration of formamide, or raising the hybridization temperature. In alternative aspects, nucleic acids of the invention are defined by their ability to hybridize under various stringency conditions (e.g., high, medium, and low), as set forth herein.

In alternative embodiments, hybridization under high stringency conditions comprises conditions of about 50% formamide at about 37° C. to 42° C. In alternative embodiments, reduced stringency conditions comprise conditions of about 35% to 25% formamide at about 30° C. to 35° C. In one aspect, hybridization occurs under high stringency conditions, e.g., at 42° C. in 50% formamide, 5×SSPE, 0.3% SDS and 200 μg/ml sheared and denatured salmon sperm DNA. In one aspect, hybridization occurs under these reduced stringency conditions, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

In alternative aspects, nucleic acids of the invention as defined by their ability to hybridize under stringent conditions to an exemplary nucleic acid of the invention (e.g., the exemplary SEQ ID NO:1, SEQ ID NO:9, SEQ ID NO:17, SEQ ID NO:24, SEQ ID NO:31); e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800, 850, 900, 950, 1000, or more, residues in length. Nucleic acids shorter than full length are also included. These nucleic acids can be useful as, e.g., hybridization probes, labeling probes, PCR oligonucleotide probes, iRNA (siRNA or miRNA, single or double stranded), antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C. Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 μg/ml sheared and denatured salmon sperm DNA). In one aspect, nucleic acids of the invention are defined by their ability to hybridize under reduced stringency conditions comprising 35% formamide at a reduced temperature of 35° C.

In alternative embodiments, nucleic acid hybridization reactions comprise conditions used to achieve a particular level of stringency and can vary depending on the nature of the nucleic acids being hybridized. For example, the length, degree of complementarity, nucleotide sequence composition (e.g., GC v. AT content) and nucleic acid type (e.g., RNA v. DNA) of the hybridizing regions of the nucleic acids can be considered in selecting hybridization conditions. An additional consideration is whether one of the nucleic acids is immobilized, for example, on a filter.

In alternative embodiments, nucleic acid hybridization reactions are carried out under conditions of low stringency, moderate stringency or high stringency. Any hybridization reaction of the invention can be defined as comprising a wash, e.g., for 30 minutes at room temperature in a buffer, e.g., a 1×SET (150 mM NaCl, 20 mM Tris hydrochloride, pH 7.8, 1 mM Na₂EDTA) comprising 0.5% SDS, followed by a 30 minute wash in fresh buffer, e.g., in 1×SET. In one aspect, hybridization conditions comprise a wash step comprising a wash for 30 minutes at room temperature in a solution comprising 1×150 mM NaCl, 20 mM Tris hydrochloride, pH 7.8, 1 mM Na₂EDTA, 0.5% SDS, followed by a wash in fresh solution.

In alternative embodiments, nucleic acid hybridization reactions comprise use of a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10×Denhardt's and 0.5 mg/ml polyriboadenylic acid. Approximately 2×10⁷ cpm (specific activity 4-9×10⁸ cpm/ug) of ³²P end-labeled oligonucleotide probe are then added to the solution. After 12-16 hours of incubation, the membrane is washed for 30 minutes at room temperature in 1×SET (150 mM NaCl, 20 mM Tris hydrochloride, pH 7.8, 1 mM Na₂EDTA) containing 0.5% SDS, followed by a 30 minute wash in fresh 1×SET at Tₘ-10° C. for the oligonucleotide probe. The membrane is then exposed to auto-radiographic film for detection of hybridization signals.

Following hybridization, a filter can be washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content) and the nucleic acid type (e.g., RNA versus. DNA). Examples of progressively higher stringency condition washes that can be used are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes. Some other examples are given below.

Nucleic acids which have hybridized to the probe can be identified by autoradiography or other conventional techniques.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

Oligonucleotides Probes and Methods for Using them

The invention also provides nucleic acid probes that can be used, e.g., for identifying nucleic acids encoding a polypeptide with KsdA, CxgA, CxgB, CxgC or CxgD (SEQ ID NO:2, SEQ ID NO:10 (and SEQ ID NO:11), SEQ ID NO:18, SEQ ID NO:25, SEQ ID NO:32, respectively) enzyme activity. In alternative embodiments, a probe of the invention can be at least about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 150 or about 10 to 50, about 20 to 60 about 30 to 70, consecutive bases of the sequence of a nucleic acid of the invention. The probes identify a nucleic acid by binding and/or hybridization. The probes can be used in arrays of the invention, see discussion below, including, e.g., capillary arrays. The probes of the invention can also be used to isolate other nucleic acids or polypeptides.

The isolated nucleic acids of the invention, the sequences complementary thereto, or a fragment comprising at least 10, 15, 20, 25, 30, 35, 40, 50, 75, 100, 150, 200, 300, 400, or 500 consecutive bases of one of the sequences of the invention, or the sequences complementary thereto may also be used as probes to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences from which are present therein.

Where necessary, conditions which permit the probe to specifically hybridize to complementary sequences may be determined by placing the probe in contact with complementary sequences from samples known to contain the complementary sequence as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids.

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product.

Many methods for using the labeled probes to detect the presence of complementary nucleic acids in a sample are familiar to those skilled in the art. These include Southern Blots, Northern Blots, colony hybridization procedures and dot blots. Protocols for each of these procedures are provided in Ausubel et al. Current Protocols in Molecular Biology, John Wiley 503 Sons, Inc. (1997) and Sambrook et al., Molecular Cloning: A Laboratory Manual 2nd Ed., Cold Spring Harbor Laboratory Press (1989.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). Typically, the probes comprise oligonucleotides. In one aspect, the amplification reaction may comprise a PCR reaction. PCR protocols are described in Ausubel and Sambrook, supra. Alternatively, the amplification may comprise a ligase chain reaction, 3SR, or strand displacement reaction. (See Barany, F., “The Ligase Chain Reaction in a PCR World”, PCR Methods and Applications 1:5-16, 1991; E. Fahy et al., “Self-sustained Sequence Replication (3SR): An Isothermal Transcription-based Amplification System Alternative to PCR”, PCR Methods and Applications 1:25-33, 1991; and Walker G. T. et al., “Strand Displacement Amplification—an Isothermal in vitro DNA Amplification Technique”, Nucleic Acid Research 20:1691-1696, 1992). In such procedures, the nucleic acids in the sample are contacted with the probes, the amplification reaction is performed and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tₘ, is the temperature (under defined ionic strength and pH) at which 50% of the target sequence hybridizes to a perfectly complementary probe. Very stringent conditions are selected to be equal to or about 5° C. lower than the Tₘ for a particular probe. The melting temperature of the probe may be calculated using the following formulas:

For probes between 14 and 70 nucleotides in length the melting temperature (Tₘ) is calculated using the formula: Tₘ=81.5+16.6(log [Na+])+0.41(fraction G+C)−(600/N) where N is the length of the probe.

If the hybridization is carried out in a solution containing formamide, the melting temperature may be calculated using the equation: Tₘ=81.5+16.6(log [Na+])+0.41(fraction G+C)−(0.63% formamide)−(600/N) where N is the length of the probe.

Prehybridization may be carried out in 6×SSC, 5×Denhardt's reagent, 0.5% SDS, 100 μg/ml denatured fragmented salmon sperm DNA or 6×SSC, 5×Denhardt's reagent, 0.5% SDS, 100 μg/ml denatured fragmented salmon sperm DNA, 50% formamide. The formulas for SSC and Denhardt's solutions are listed in Sambrook et al., supra.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tₘ. For shorter probes, such as oligonucleotide probes, the hybridization may be conducted at 5-10° C. below the Tₘ. In one aspect, for hybridizations in 6×SSC, the hybridization is conducted at approximately 68° C. Usually, for hybridizations in 50% formamide containing solutions, the hybridization is conducted at approximately 42° C.

Inhibiting Expression of KsdA-, CxgA-, CxgB-, CxgC- and/or CxgD

The invention provides nucleic acids complementary to (e.g., antisense sequences to) the nucleic acids encoding KsdA, CxgA, CxgB, CxgC or CxgD, including nucleic acids comprising antisense, iRNA, ribozymes. Nucleic acids used to practice the invention can comprise antisense sequences capable of inhibiting the transport, splicing or transcription of KsdA, CxgA, CxgB, CxgC or CxgD-encoding genes. In alternative embodiments, the expression of a message (mRNA) of a KsdA, CxgA, CxgB, CxgC and/or CxgD-encoding nucleic acid is deleted or disrupted by an antisense, ribozyme and/or RNAi specific for a message (mRNA) of a KsdA, CxgA, CxgB, CxgC and/or CxgD-encoding nucleic acid.

In alternative embodiments, inhibition can be effected through the targeting of genomic DNA or transcripts (mRNA). The transcription or function of targeted nucleic acid can be inhibited, for example, by hybridization and/or cleavage. In alternative embodiments, oligonucleotides which are able to bind KsdA, CxgA, CxgB, CxgC and/or CxgD-encoding nucleic acid, gene or message to prevent or inhibit the production or function of these polypeptides are used. The association can be through sequence specific hybridization.

In alternative embodiments, inhibitors that can be used include oligonucleotides which cause inactivation or cleavage of ksdA, cxgA, cxgB, cxgC and/or cxgD (SEQ ID NO:1, SEQ ID NO:9, SEQ ID NO:17, SEQ ID NO:24 and SEQ ID NO:31, respectively) message. The oligonucleotide can have enzyme activity which causes such cleavage, such as ribozymes. The oligonucleotide can be chemically modified or conjugated to an enzyme or composition capable of cleaving the complementary nucleic acid. A pool of many different such oligonucleotides can be screened for those with the desired activity. Thus, the invention provides various compositions for the inhibition of KsdA-, CxgA-, CxgB-, CxgC- and/or CxgD expression on a nucleic acid and/or protein level, e.g., antisense, iRNA (e.g., siRNA, miRNA) and ribozymes comprising ksdA, cxgA, cxgB, cxgC and/or cxgD sequences of the invention and antibodies of the invention (including antibodies that inhibit the expression or activity of KsdA, CxgA, CxgB, CxgC and/or CxgD).

Antisense Oligonucleotides

The invention provides antisense oligonucleotides capable of binding ksdA, cxgA, cxgB, cxgC and/or cxgD message which, in one aspect, can inhibit KsdA, CxgA, CxgB, CxgC and/or CxgD activity by targeting mRNA. Strategies for designing antisense oligonucleotides are well described in the scientific and patent literature, and the skilled artisan can design such ksdA, cxgA, cxgB, cxgC and/or cxgD (SEQ ID NO:1, SEQ ID NO:9, SEQ ID NO:17, SEQ ID NO:24 and SEQ ID NO:31, respectively) oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense ammonia lyase, e.g., phenylalanine ammonia lyase, tyrosine ammonia lyase and/or histidine ammonia lyase enzyme sequences of the invention (see, e.g., Gold (1995) J. of Biol. Chem. 270:13581-13584).

Inhibitory Ribozymes

The invention provides ribozymes capable of binding ksdA, cxgA, cxgB, cxgC and/or cxgD (SEQ ID NO:1, SEQ ID NO:9, SEQ ID NO:17, SEQ ID NO:24 and SEQ ID NO:31, respectively) message. These ribozymes can inhibit KsdA, CxgA, CxgB, CxgC and/or CxgD activity by, e.g., targeting mRNA. Strategies for designing ribozymes and selecting the ksdA, cxgA, cxgB, cxgC and/or cxgD-specific antisense sequences for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The ribozyme of the invention, e.g., an enzymatic ribozyme RNA molecule, can be formed in a hammerhead motif, a hairpin motif, as a hepatitis delta virus motif, a group I intron motif and/or an RNaseP-like RNA in association with an RNA guide sequence. Examples of hammerhead motifs are described by, e.g., Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting. Those skilled in the art will recognize that a ribozyme of the invention, e.g., an enzymatic RNA molecule of this invention, can have a specific substrate binding site complementary to one or more of the target gene RNA regions. A ribozyme of the invention can have a nucleotide sequence within or surrounding that substrate binding site which imparts an RNA cleaving activity to the molecule.

RNA Interference (RNAi)

In one aspect, the invention provides an RNA inhibitory molecule, a so-called “RNAi” molecule, comprising a ksdA, cxgA, cxgB, cxgC and/or cxgD (SEQ ID NO:1, SEQ

ID NO:9, SEQ ID NO:17, SEQ ID NO:24 and SEQ ID NO:31, respectively) sequence of the invention. The RNAi molecule comprises a double-stranded RNA (dsRNA) molecule. The RNAi molecule, e.g., siRNA and/or miRNA, can inhibit expression of a ksdA, cxgA, cxgB, cxgC and/or cxgD gene. In one aspect, the RNAi molecule, e.g., siRNA and/or miRNA, is about 15, 16, 17, 18, 19, 20, 21, 22, 23, 24, 25 or more duplex nucleotides in length.

While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's molecules, e.g., siRNA and/or miRNA, of the invention. In one aspect, the micro-inhibitory RNA (miRNA) inhibits translation, and the siRNA inhibits transcription. The process may be practiced in vitro, ex vivo or in vivo. In one aspect, the RNAi molecules of the invention can be used to generate a loss-of-function mutation in a cell, an organ or an animal. Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

### Transgenic Non-Human Animals

The invention provides transgenic non-human animals comprising a nucleic acid, a polypeptide (e.g., a KsdA, CxgA, CxgB, CxgC and/or CxgD), an expression cassette or vector or a transfected or transformed cell of the invention. The invention also provides methods of making and using these transgenic non-human animals.

The transgenic non-human animals can be, e.g., goats, rabbits, sheep, pigs (including all swine, hogs and related animals), cows, rats and mice, comprising the nucleic acids of the invention. These animals can be used, e.g., as in vivo models to study KsdA, CxgA, CxgB, CxgC and/or CxgD activity, or, as models to screen for agents that change KsdA, CxgA, CxgB, CxgC and/or CxgD activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice. U.S. Pat. No. 6,187,992, describes making and using a transgenic mouse.

“Knockout animals” or “Knockout cells” can also be used to practice the methods of the invention. For example, in one aspect, the transgenic or modified animals or cells of the invention comprise a “knockout animal,” or knockout cell, e.g., a knockout mouse or mouse cell, engineered not to express an endogenous ksdA, cxgA, cxgB, cxgC and/or cxgD (SEQ ID NO:1, SEQ ID NO:9, SEQ ID NO:17, SEQ ID NO:24 and SEQ ID NO:31, respectively) gene, and optionally the knocked out gene is replaced with a gene expressing another (e.g., a heterologous) KsdA, CxgA, CxgB, CxgC and/or CxgD, or, a fusion protein comprising a KsdA, CxgA, CxgB, CxgC and/or CxgD, or comparable encoding gene have lower, e.g., very low, levels of expression as compared to wild type.

### Transgenic Plants and Seeds

The invention provides transgenic plants and seeds comprising a nucleic acid, a polypeptide (e.g., KsdA, CxgA, CxgB, CxgC and/or CxgD), an expression cassette or vector or a transfected or transformed cell of the invention. The invention also provides plant products, e.g., oils, seeds, leaves, extracts and the like, comprising a nucleic acid and/or a polypeptide (e.g., k KsdA, CxgA, CxgB, CxgC and/or CxgD) of the invention. The invention also provides plant products, e.g., oils, seeds, leaves, extracts and the like, comprising a nucleic acid and/or a polypeptide (e.g., KsdA, CxgA, CxgB, CxgC and/or CxgD) of the invention.

In alternative embodiments, the invention provides transgenic plants and seeds comprising where nucleic acids encoding KsdA, CxgA, CxgB, CxgC and/or CxgD have been deleted or disabled.

The transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. No. 6,309,872.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's KsdA, CxgA, CxgB, CxgC and/or CxgD production is regulated by endogenous transcriptional or translational control elements.

The invention also provides “knockout plants” where insertion of gene sequence by, e.g., homologous recombination, has disrupted the expression of the endogenous gene, e.g., the host cell's equivalent of ksdA, cxgA, cxgB, cxgC and/or cxgD. Means to generate “knockout” plants are well-known in the art, see, e.g., Strepp (1998) Proc Natl. Acad. Sci. USA 95:4368-4373; Miao (1995) Plant J 7:359-365.

The nucleic acids and polypeptides of the invention are expressed in or inserted in any prokaryotic, eukaryotic or plant cell, plant or seed, including e.g., insertion and/or expression in a ksdA, cxgA, cxgB, cxgC and/or cxgD (SEQ ID NO:1, SEQ ID NO:9, SEQ ID NO:17, SEQ ID NO:24 and SEQ ID NO:31, respectively) “knockout” version. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

The invention also provides for transgenic plants to be used for producing large amounts of the polypeptides (e.g., a polypeptide or antibody) of the invention. For example, see Palmgren (1997) Trends Genet. 13:348; Chong (1997) Transgenic Res. 6:289-296, producing human milk protein beta-casein in transgenic potato plants using an auxin-inducible, bidirectional mannopine synthase (mas1′,2′) promoter with Agrobacterium tumefaciens-mediated leaf disc transformation methods.

Using known procedures, one of skill can screen for plants of the invention by detecting the increase or decrease of transgene mRNA or protein in transgenic plants. Means for detecting and quantitation of mRNAs or proteins are well known in the art.

### Polypeptides and Peptides

In one aspect, the invention provides isolated, synthetic or recombinant polypeptides having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to: SEQ ID NO:2, and enzymatically active fragments thereof, and having a KsdA polypeptide or a 3-ketosteroid-Δ1-dehydrogenase activity; SEQ ID NO:10 (and SEQ ID NO:11), and enzymatically active fragments thereof, and having a CxgA polypeptide or an acetyl CoA-acetyltransferase/thiolase activity; SEQ ID NO:18, and enzymatically active fragments thereof, and having a CxgB polypeptide or a DNA-binding protein activity; SEQ ID NO:25, and enzymatically active fragments thereof, and having a CxgC polypeptide or a DNA-binding protein activity; and, SEQ ID NO:32, and enzymatically active fragments thereof, and having a CxgD polypeptide or a TetR-like regulatory protein/KstR activity (all of these polypeptides are polypeptides of the invention). In one embodiment, the invention also provides polypeptides in the form of antibodies that can bind to these polypeptides of the invention.

In one embodiment, polypeptides of the invention also encompass amino acid sequences comprising a sequence of an exemplary polypeptide of the invention (e.g., SEQ ID NO:2, SEQ ID NO:12, SEQ ID NO:13, SEQ ID NO:14, SEQ ID NO:15) but having at least one conservative substitution of an amino acid residue but still retaining its activity (e.g., a 3-ketosteroid-Δ1-dehydrogenase activity, or KsdA, CxgA, CxgB, CxgC or CxgD activity), wherein optionally conservative substitution comprises replacement of an aliphatic amino acid with another aliphatic amino acid; replacement of a serine with a threonine or vice versa; replacement of an acidic residue with another acidic residue; replacement of a residue bearing an amide group with another residue bearing an amide group; exchange of a basic residue with another basic residue; or, replacement of an aromatic residue with another aromatic residue, or a combination thereof, and optionally the aliphatic residue comprises Alanine, Valine, Leucine, Isoleucine or a synthetic equivalent thereof; the acidic residue comprises Aspartic acid, Glutamic acid or a synthetic equivalent thereof; the residue comprising an amide group comprises Aspartic acid, Glutamic acid or a synthetic equivalent thereof; the basic residue comprises Lysine, Arginine or a synthetic equivalent thereof; or, the aromatic residue comprises Phenylalanine, Tyrosine or a synthetic equivalent thereof.

Polypeptides of the invention can also be shorter than the full length of exemplary polypeptides. In alternative aspects, the invention provides polypeptides (peptides, fragments) ranging in size between about 5 and the full length of a polypeptide of the invention; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, or more residues. Peptides of the invention (e.g., a subsequence of an exemplary polypeptide of the invention) can be useful as, e.g., labeling probes, antigens, toleragens, motifs, ammonia lyase, e.g., phenylalanine ammonia lyase, tyrosine ammonia lyase and/or histidine ammonia lyase enzyme active sites (e.g., “catalytic domains”), signal sequences and/or prepro domains.

In one embodiment, “amino acid” or “amino acid sequence” encompasses an oligopeptide, peptide, polypeptide, or protein sequence, or to a fragment, portion, or subunit of any of these and to naturally occurring or synthetic molecules. In one embodiment, “amino acid” or “amino acid sequence” includes an oligopeptide, peptide, polypeptide, or protein sequence, or to a fragment, portion, or subunit of any of these, and to naturally occurring or synthetic molecules. In one embodiment, “polypeptide” encompasses amino acids joined to each other by peptide bonds or modified peptide bonds, i.e., peptide isosteres and may contain modified amino acids other than the 20 gene-encoded amino acids. The polypeptides may be modified by either natural processes, such as post-translational processing, or by chemical modification techniques which are well known in the art. Modifications can occur anywhere in the polypeptide, including the peptide backbone, the amino acid side-chains and the amino or carboxyl termini. In alternative embodiments, the same type of modification may be present in the same or varying degrees at several sites in a given polypeptide. Also a given polypeptide may have many types of modifications. In alternative embodiments, modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, glucan hydrolase processing, phosphorylation, prenylation, racemization, selenoylation, sulfation and transfer-RNA mediated addition of amino acids to protein such as arginylation. (See Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983)). The peptides and polypeptides of the invention also include all “mimetic” and “peptidomimetic” forms, as described in further detail, below.

In one embodiment, “isolated” means that a material, e.g., a polypeptide of the invention or a product made by a method of the invention, e.g., AD, ADD, X1 or X2, is removed from its original environment, e.g., the natural environment if it is naturally occurring. For example, a naturally-occurring polynucleotide or polypeptide or product of a process that is present in a living animal is not isolated, but the same polynucleotide or polypeptide or product of a process separated from some or all of the coexisting materials in the natural system, is isolated. In one embodiment, polynucleotides are part of a vector and/or such polynucleotides or polypeptides could be part of a composition and still be isolated in that such vector or composition is not part of its natural environment.

In one embodiment, the term “purified”, e.g., referring to a polypeptide of the invention or a product made by a method of the invention, e.g., AD, ADD, X1 or X2, does not require absolute purity; rather, it is intended as a relative definition. For example, in one embodiment, when practicing a method of this invention, a cell (e.g., that underexpresses as compared to a wild type cell or does not express any one, or several of, or all of KsdA, CxgA, CxgB, CxgC or CxgD-encoding nucleic acids and/or KsdA, CxgA, CxgB, CxgC or CxgD polypeptides in the cell) produces (generates) an androstenedione (AD) of relative greater purity, or substantially free of androstadienedione (ADD), 20-(hydroxymethyl) pregna-4-en-3-one and/or 20-(hydroxymethyl)pregna-1,4-dien-3-one by at least about 1.0%, 2.0%, 3.0%, 4.0%, 5.0%, 10.0%, 10.5%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll (1993) DNA Cell. Biol., 12:441-53.

In alternative embodiments, peptides and polypeptides of the invention include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants or members of a genus of polypeptides of the invention routine experimentation will determine whether a mimetic is within the scope of the invention, i.e., that its structure and/or function is not substantially altered. Thus, in one aspect, a mimetic composition is within the scope of the invention if it has a KsdA, CxgA, CxgB, CxgC or CxgD activity.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R— or S— form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

Signal Sequences, Prepro and Catalytic Domains

In alternative embodiments, polypeptides of the invention comprise signal sequences (e.g., signal peptides (SPs)), prepro domains and catalytic domains (CDs). The SPs, prepro domains and/or CDs can be isolated, synthetic or recombinant peptides or can be part of a fusion protein, e.g., as a heterologous domain in a chimeric protein. The invention provides nucleic acids encoding these catalytic domains (CDs), prepro domains and signal sequences (SPs, e.g., a peptide having a sequence comprising/consisting of amino terminal residues of a polypeptide of the invention).

The invention provides isolated, synthetic or recombinant signal sequences (e.g., signal peptides) consisting of or comprising a sequence as set forth in residues 1 to 11, 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 40, 1 to 41, 1 to 42, 1 to 43, 1 to 44, 1 to 45, 1 to 46, 1 to 47, 1 to 48, 1 to 49, 1 to 50, or more, of a polypeptide of the invention. In one aspect, the invention provides signal sequences comprising the first 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more amino terminal residues of a polypeptide of the invention.

Methods for identifying “prepro” domain sequences and signal sequences are well known in the art, see, e.g., Van de Ven (1993) Crit. Rev. Oncog. 4(2):115-136. For example, to identify a prepro sequence, the protein is purified from the extracellular space and the N-terminal protein sequence is determined and compared to the unprocessed form.

The invention includes polypeptides with or without a signal sequence and/or a prepro sequence. The invention includes polypeptides with heterologous signal sequences and/or prepro sequences. The prepro sequence (including a sequence of the invention used as a heterologous prepro domain) can be located on the amino terminal or the carboxy terminal end of the protein. The invention also includes isolated, synthetic or recombinant signal sequences, prepro sequences and catalytic domains (e.g., “active sites”) comprising sequences of the invention. The polypeptide comprising a signal sequence of the invention can be a polypeptide of the invention or another ammonia lyase, e.g., phenylalanine ammonia lyase, tyrosine ammonia lyase and/or histidine ammonia lyase enzyme or another enzyme or other polypeptide.

### Screening Methodologies and “On-Line” Monitoring Devices

In practicing the methods of the invention, a variety of apparatus and methodologies can be used to in conjunction with the polypeptides and nucleic acids of the invention, e.g., to screen polypeptides for KsdA, CxgA, CxgB, CxgC or CxgD activity, to screen compounds as potential modulators, e.g., activators or inhibitors, of KsdA, CxgA, CxgB, CxgC or CxgD, for antibodies that bind to a polypeptide of the invention, for nucleic acids that hybridize to a nucleic acid of the invention, to screen for cells expressing a polypeptide of the invention and the like. In addition to the array formats described in detail below for screening samples, alternative formats can also be used to practice the methods of the invention. Such formats include, for example, mass spectrometers, chromatographs, e.g., high-throughput HPLC and other forms of liquid chromatography, and smaller formats, such as 1536-well plates, 384-well plates and so on. High throughput screening apparatus can be adapted and used to practice the methods of the invention, see, e.g., U.S. Patent Application No. 20020001809.

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

Capillary Arrays

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays, such as the GIGAMATRIX™, Diversa Corporation, San Diego, Calif.; and arrays described in, e.g., U.S. Patent Application No. 20020080350 A1; WO 0231203 A; WO 0244336 A, provide an alternative apparatus for holding and screening samples. In one aspect, the capillary array includes a plurality of capillaries formed into an array of adjacent capillaries, wherein each capillary comprises at least one wall defining a lumen for retaining a sample. The lumen may be cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. Additionally, the capillary array can include interstitial material disposed between adjacent capillaries in the array, thereby forming a solid planar device containing a plurality of through-holes.

A capillary array can be formed of any number of individual capillaries, for example, a range from 100 to 4,000,000 capillaries. Further, a capillary array having about 100,000 or more individual capillaries can be formed into the standard size and shape of a MICROTITER® plate for fitment into standard laboratory equipment. The lumens are filled manually or automatically using either capillary action or microinjection using a thin needle. Samples of interest may subsequently be removed from individual capillaries for further analysis or characterization. For example, a thin, needle-like probe is positioned in fluid communication with a selected capillary to either add or withdraw material from the lumen.

In a single-pot screening assay, the assay components are mixed yielding a solution of interest, prior to insertion into the capillary array. The lumen is filled by capillary action when at least a portion of the array is immersed into a solution of interest. Chemical or biological reactions and/or activity in each capillary are monitored for detectable events. A detectable event is often referred to as a “hit”, which can usually be distinguished from “non-hit” producing capillaries by optical detection. Thus, capillary arrays allow for massively parallel detection of “hits”.

In a multi-pot screening assay, a polypeptide or nucleic acid, e.g., a ligand, can be introduced into a first component, which is introduced into at least a portion of a capillary of a capillary array. An air bubble can then be introduced into the capillary behind the first component. A second component can then be introduced into the capillary, wherein the second component is separated from the first component by the air bubble. The first and second components can then be mixed by applying hydrostatic pressure to both sides of the capillary array to collapse the bubble. The capillary array is then monitored for a detectable event resulting from reaction or non-reaction of the two components.

In a binding screening assay, a sample of interest can be introduced as a first liquid labeled with a detectable particle into a capillary of a capillary array, wherein the lumen of the capillary is coated with a binding material for binding the detectable particle to the lumen. The first liquid may then be removed from the capillary tube, wherein the bound detectable particle is maintained within the capillary, and a second liquid may be introduced into the capillary tube. The capillary is then monitored for a detectable event resulting from reaction or non-reaction of the particle with the second liquid.

Arrays, or “Biochips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a ksdA, cxgA, cxgB, cxgC and/or cxgD gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins. The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Enzyme Activity Screening Protocols

In some embodiments, practicing the methods and compositions of this invention comprises screening polypeptides for KsdA, CxgA, CxgB, CxgC or CxgD activity; screening compounds as potential modulators, e.g., activators or inhibitors, of KsdA, CxgA, CxgB, CxgC or CxgD polypeptides; and/or screening for antibodies that bind to a polypeptide of the invention, and in some embodiments, inhibit the polypeptide's activity. In practicing these embodiments, any method, process or protocol for determining KsdA, CxgA, CxgB, CxgC or CxgD activity can be used.

For example exemplary protocols for determining whether a polypeptide has a KsdA activity are described e.g., by van der Geize, et al. (2000) Applied and Environm. Microbiol. 66(5):2029-2036; van der Geize, et al. (2001) FEMS Microbiol Lett. 205(2):197-202); van der Geize, et al. (2002) Microbiology 148 (Pt 10):3285-3292; Knol, et al. (2008) Biochem J. 410(2):339-346.

Exemplary protocols for determining whether a polypeptide has a CxgA, CxgB, CxgC or CxgD activity include defining the activity of the polypeptide based a cell's phenotype after deletion or disabling of the polypeptide's activity, as described herein. For example, a polypeptide has a KsdA, CxgA, CxgB, CxgC or CxgD activity if it can complement (e.g., replace, restore) a wild type phenotype after “knocking out” the corresponding KsdA, CxgA, CxgB, CxgC or CxgD gene, or otherwise deleting or disabling the corresponding message or polypeptide. If by adding the polypeptide in question back to the “disabled” cell a wild type phenotype is restored, then that polypeptide has the requisite activity, e.g., enzyme or binding activity. For example, if the KsdA gene and/or KsdA polypeptide is deleted or otherwise disabled in a cell, the cell then lacks a 3-ketosteroid-Δ1-dehydrogenase activity; and if adding a polypeptide in question back to that modified cell restores the 3-ketosteroid-Δ1-dehydrogenase activity, then that polypeptide screens positively for 3-ketosteroid-Δ1-dehydrogenase activity and a KsdA activity. Similarly, if the CxgA gene and/or CxgA polypeptide is deleted or otherwise disabled in a cell, the cell then lacks an acetyl CoA-acetyltransferase/thiolase activity; and if adding a polypeptide in question back to that modified cell restores the acetyl CoA-acetyltransferase/thiolase activity, then that polypeptide screens positively for acetyl CoA-acetyltransferase/thiolase activity and a CxgA activity; and so forth.

### Antibodies and Antibody-Based Screening Methods

The invention provides isolated, synthetic or recombinant antibodies that specifically bind to a polypeptide of the invention. These antibodies can be used to isolate, identify or quantify KsdA, CxgA, CxgB, CxgC or CxgD of the invention or related polypeptides. These antibodies can be used to isolate other polypeptides within the scope the invention or other related KsdA, CxgA, CxgB, CxgC or CxgD proteins. The antibodies can be designed to bind to an active site of KsdA, CxgA, CxgB, CxgC or CxgD. Thus, the invention provides methods of inhibiting KsdA, CxgA, CxgB, CxgC or CxgD using the antibodies of the invention.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The invention provides subsequences of polypeptides of the invention, e.g., enzymatically active or immunogenic fragments of the enzymes of the invention, including immunogenic fragments of a polypeptide of the invention. The invention provides compositions comprising a polypeptide or peptide of the invention and adjuvants or carriers and the like.

The antibodies can be used in immunoprecipitation, staining, immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The polypeptides of the invention or fragments comprising at least 5, 10, 15, 20, 25, 30, 35, 40, 50, 75, 100, or 150 consecutive amino acids thereof, may also be used to generate antibodies which bind specifically to the polypeptides or fragments. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention, or fragments comprising at least 5, 10, 15, 20, 25, 30, 35, 40, 50, 75, 100, or 150 consecutive amino acids thereof.

In immunoaffinity procedures, the antibody is attached to a solid support, such as a bead or other column matrix. The protein preparation is placed in contact with the antibody under conditions in which the antibody specifically binds to one of the polypeptides of the invention, or fragment thereof. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays and Western Blots.

Polyclonal antibodies generated against the polypeptides of the invention, or fragments comprising at least 5, 10, 15, 20, 25, 30, 35, 40, 50, 75, 100, or 150 consecutive amino acids thereof can be obtained by direct injection of the polypeptides into an animal or by administering the polypeptides to an animal, for example, a nonhuman. The antibody so obtained can bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique (Kohler and Milstein, Nature, 256:495-497, 1975), the trioma technique, the human B-cell hybridoma technique (Kozbor et al., Immunology Today 4:72, 1983) and the EBV-hybridoma technique (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to the polypeptides of the invention, or fragments comprising at least 5, 10, 15, 20, 25, 30, 35, 40, 50, 75, 100, or 150 consecutive amino acids thereof. Alternatively, transgenic mice may be used to express humanized antibodies to these polypeptides or fragments thereof.

Antibodies generated against the polypeptides of the invention, or fragments comprising at least 5, 10, 15, 20, 25, 30, 35, 40, 50, 75, 100, or 150 consecutive amino acids thereof may be used in screening for similar polypeptides from other organisms and samples. In such techniques, polypeptides from the organism are contacted with the antibody and those polypeptides which specifically bind the antibody are detected. Any of the procedures described above may be used to detect antibody binding. One such screening assay is described in “Methods for Measuring Cellulase Activities”, Methods in Enzymology, Vol 160, pp. 87-116.

### Kits

The invention provides kits comprising the compositions, e.g., KsdA, CxgA, CxgB, CxgC or CxgD of the invention and, e.g., nucleic acids, expression cassettes, vectors, cells, transgenic seeds or plants or plant parts, polypeptides (e.g., KsdA, CxgA, CxgB, CxgC or CxgD) and/or antibodies of the invention. The kits also can contain instructional material teaching the methodologies and industrial uses of the invention, as described herein.

The following examples are intended to illustrate, but not to limit, the invention. While the procedures described in the examples are typical of those that can be used to carry out certain aspects of the invention, other procedures known to those skilled in the art can also be used.

### EXAMPLES

### Example 1

### Making and Using Exemplary Genes and Host Cells of the Invention

This example describes making and using exemplary host cells of the invention to make 1,4-androstadiene-3,17-dione (ADD) and related pathway compounds, including 20-(hydroxymethyl)pregna-4-en-3-one and 20-(hydroxymethyl)pregna-1,4-dien-3-one.

In one aspect, the invention provides modified host cell of the invention is a bacterial cell, e.g., a Mycobacterium strains, such as a Mycobacterium strain designated B3683 (see e.g., Perez et al. (1995) Biotechnology Letters 17(11):1241-1246) and B3805 (see, e.g., Golańska (1998) Acta Microbiol Pol. 47(4):335-343). Mycobacterium B3683 was generated from a soil isolate by mutagenesis to eliminate the complete degradation of phytosterols and to enable the production of ADD and AD. As the B3683 strain produces significantly more ADD than AD, Mycobacterium B3805 was derived from B3683 by mutagenesis to reduce ADD production in favor of AD. Mycobacterium B3805 remains uncharacterized as to its mutations and is reported to still produce small amounts of ADD; see e.g., Goren (1983) J. Steroid Biochem. 19(6):1789-1797.

In the original description of strains B3683 and B3805, see e.g., Marshek (1972) supra, it was also noted that 20-(hydroxymethyl)pregna-1,4-dien-3-one (compound X2) was produced. Compound X2 is thought to be a terminal side product resulting from the incomplete removal of the alkyl side chain of phytosterols. The inventors determined that this strain is capable of producing Compound X1, which is converted to Compound X2 by the same 3-ketosteroid-Δ1-dehydrogenase activity that converts AD to ADD.

Strain Improvement

### 1) Characterization of Organism Used as Basis for Strain Development

Mycobacterium B3683 (ATCC 29472) was obtained from the American Type Culture Collection (Manassas, Va.) and streaked onto MYM agar plates to obtain single colonies. Three different colony morphologies or morphotypes were seen, a phenomenon previously described for many Mycobacterium species. The individual morphotypes were selected and serially passaged to obtain pure cultures of each.

Further characterization of each then demonstrated that one morphotype, variant 2, was most amenable for culturing due to its confluent growth characteristics in liquid medium. In addition, each of the variants was tested for its ability to serve as a genetic recipient of the EZ::TN™<R6Kγori/KAN-2> TRANSPOSOME™ (Epicentre, Madison, Wis.) by preparing electrocompetent cells, electroporating and selecting for kanamycin-resistant clones. Again, morphotype variant 2 was determined to be the most amenable to this genetic manipulation and was selected as background for further generation of mutants and identification of relevant genes.

### 2) Generation of Mycobacterium B3683 Transposon Mutants

Electrocompetent cells of variant 2 were electroporated with the EZ::TN <R6Kori/Kan-2> TRANSPOSOME™ and plated onto L-agar containing 50 μg/ml kanamycin. Approximately 6000 colonies were obtained from multiple electroporations. Each of the colonies were arrayed into individual wells of a 96-well plate containing 200 μl 2×YT per well, sealed with a gas-permeable membrane and grown at 30° C. for 48 hours in a HIGRO™ incubator (Genomic Solutions, Ann Arbor, Mich.) at 400 rpm with intermittent aeration. Cells were prepared for storage by addition and mixing of 20 μl glycerol and freezing at −80° C.

### 3) Identification of Mutants Unable to Convert AD to ADD

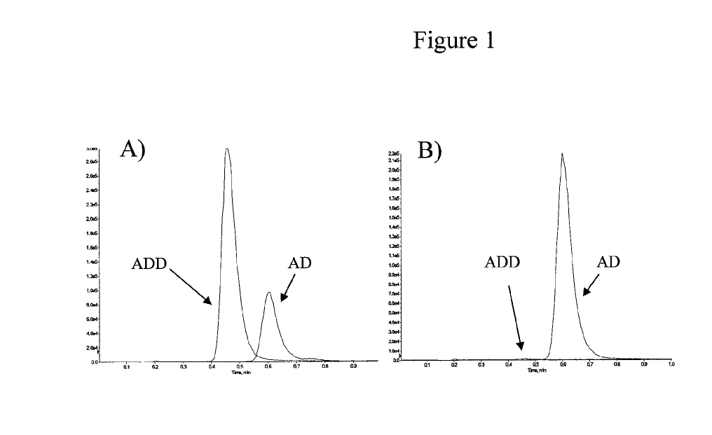

Each of the transposon mutants were assayed for their ability to convert AD to ADD (assayed as described below). From this screen, one mutant was identified as unable to convert AD to ADD, as illustrated in FIG. 1B. This mutant was retested in triplicate and determined to be completely deficient in this conversion.

FIG. 1 illustrates data from an exemplary AD to ADD conversion assay: FIG. 1A illustrates data from a random Tn5 mutant; FIG. 1B illustrates data from a ksdA Tn5 mutant, showing the absence of AD to ADD conversion. Y-axis values represent LC/MS/MS peak area responses and not absolute quantitation of product.

### 4) Identification of Gene Responsible for AD to ADD Conversion

A culture of the mutant was harvested and used to prepare chromosomal DNA by standard laboratory procedures. This DNA was digested with one of two restriction enzymes, BglII or EcoRI, to completion. After inactivation of the restriction enzymes, the digested DNAs were diluted and each incubated with T4 DNA ligase to generate circular intramolecular ligation products. Ligation products were then electroporated into E. coli strain EPI300, carrying a chromosomal copy of the pir gene, enabling the replication as a plasmid of a circular ligation product containing the EZ::Tn <R6Kori/Kan-2>TRANSPOSOME™. Kanamycin-resistant transformants were selected, clonally purified and grown to prepare transposon-containing plasmid DNA.

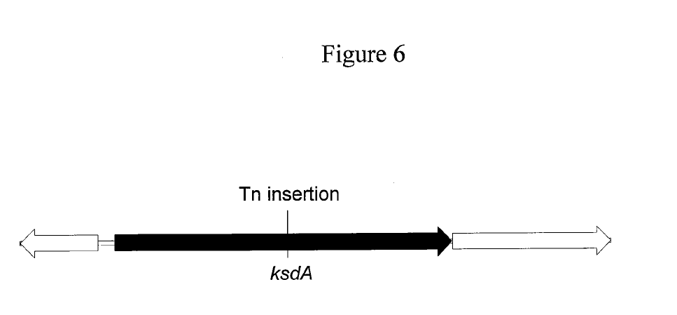

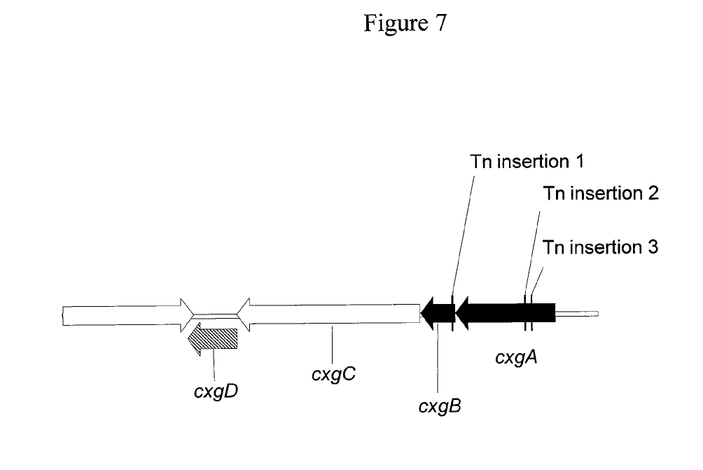

The plasmid DNAs were sequenced using primers extending outward from the ends of the known transposon sequence into uncharacterized flanking sequence. After further extension of the sequencing by primer walking, it was determined that the transposon was inserted into an open reading frame with significant homology to putative 3-ketosteroid-Δ1-dehydrogenases, as would be expected for an enzyme with the ability to convert AD to ADD, as illustrated in FIG. 6 and FIG. 7. FIG. 6 is a schematic illustration of an exemplary chromosomal site of insertion and gene organization around the 3-ketosteroid-Δ1-dehydrogenase mutation abolishing AD to ADD conversion. FIG. 7 is a schematic illustration of exemplary chromosomal sites of insertions and organization of the “cxg genes”, i.e., the cxgA, cxgB, cxgC, or cxgD genes.

For purposes of nomenclature, this gene will be referred to as ksdA (ketosteroid dehydrogenase). Only the Rhodococcus erythropolis and Comamonas testosteroni homologs had been experimentally determined to have the dehydrogenase activity; see e.g., van der Geize (2002) Microbiology 148(10):3285-3292; Horinouchi (2003) App. & Env. Microbiology 69(8):4421-4430.

### 5) Identification of Mutants Unable to Convert Cholesterol to Compound X1/X2

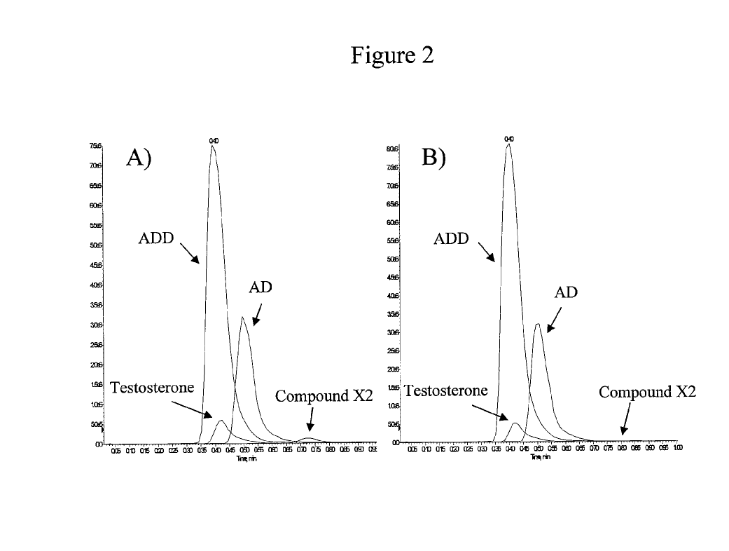

Each of the transposon mutants were assayed for their ability to convert cholesterol to products (assay as described below). Approximately half of the mutants were screened for conversion of cholesterol to AD, ADD, testosterone and compound X2. One mutant was found that produced significantly reduced levels of X2 compared to the wild-type strain, see FIG. 2 using the Tn mutant 1. FIG. 2 illustrates data from an exemplary cholesterol conversion assay (X2 only): FIG. 2A uses the random Tn5 mutant, and FIG. 2B uses the cxgB Tn5 mutant 1, showing absence of Compound X2 production. Y-axis values represent LC/MS/MS peak area responses and not absolute quantitation of product.

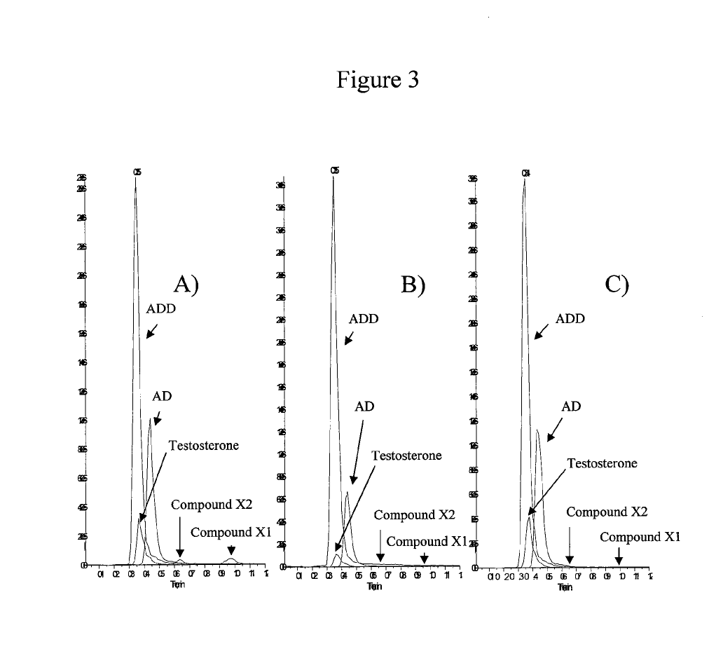

Two additional mutants were identified that produced significantly reduced levels of X1 and X2 as compared to wild-type, see FIG. 3, using Tn mutants 2 and 3. FIG. 3 illustrates data from an exemplary cholesterol conversion assay (X1 and X2), showing absence of compounds X1 and X2 production: FIG. 3A uses the random Tn5 mutant, FIG. 3B uses the cxgA Tn5 mutant 2, and FIG. 3C uses the cxgA Tn5 mutant 3. Y-axis values represent LC/MS/MS peak area responses and not absolute quantitation of product.

All three mutants were then retested in triplicate and determined to be impaired in the ability to produce X1 and X2. The Tn5 mutant in the ksdA gene described above was unable to produce ADD or compound X2 from cholesterol, confirming the defect in 3-ketosteroid-Δ1-dehydrogenase activity responsible for the conversion of X1 to X2.

### 6) Identification of Candidate Genes Responsible for Converting Cholesterol to X1/X2

As described above, plasmid DNA containing the transposon-mutagenized and adjacent chromosomal sequences was isolated from each of the mutants and sequenced. From this initial characterization, additional sequences would be useful to determine the nature of the gene or genes required for this conversion. These were obtained by hybridization of a Mycobacterium B3683 genomic fosmid library with a probe derived from the known sequence and further extension of sequencing from an isolated fosmid.

From this sequencing effort, it was determined that the transposon insertions in the three mutants were located in an operon composed of four open reading frames, see FIG. 7, also discussed above. Two of the insertions were found in the first gene of the operon and one insertion was found in the second gene of the operon. For purposes of nomenclature, the genes in the operon will be referred to as cxgA-D (compound X genes).

A BlastX search of the GenBank database showed that polypeptide CxgA (SEQ ID NO:12) had significant homology to an unidentified Mycobacterium avium paratuberculosis ORF MAP4302C as well as hypothetical acetyl CoA-acetyltransferases/thiolases, which are normally involved in the fatty acid metabolism. The polypeptide CxgB (SEQ ID NO:13) was found to have significant homology to MAP4301c from Mycobacterium avium paratuberculosis and limited homology to a number of putative DNA-binding proteins. The polypeptide CxgC (SEQ ID NO:14) showed significant homology to putative acyl-CoA dehydrogenases/FadE proteins. The polypeptide CxgD (SEQ ID NO:15) was found to have significant homology to a number of putative TetR-like regulatory proteins, including KstR, a negative regulator of steroid metabolism in Rhodococcus erythropolis. The site of insertions are illustrated in FIGS. 6 and 7, and the nucleotide and protein sequences of cxgA, cxgB, cxgC and cxgD are set forth below. The gene sequences of cxgA, cxgB, cxgC and cxgD, are set forth respectively in SEQ ID NO:8, SEQ ID NO:9, SEQ ID NO:10 and SEQ ID NO:11; and the polypeptide cxgA, cxgB, cxgC and cxgD amino acid sequences are set forth respectively in SEQ ID NO:12, SEQ ID NO:13, SEQ ID NO:14 and SEQ ID NO:15.

### 7) Deletion of Gene Responsible for Conversion of AD to ADD

To generate a targeted deletion of the ksdA gene (SEQ ID NO:1), responsible for the conversion of AD to ADD, a markerless gene replacement strategy was used as follows. One-kilobase sequences flanking either side of the ORF were generated by PCR and ligated together through an introduced Type IIS enzyme site to generate a 2 kb fragment. This fragment was then introduced into a cloning vector containing a TopoTA-cloning site and a kanamycin-resistance determinant. Into this construction, an additional fragment was introduced, containing the sacB sucrose synthase gene from B. subtilis. The resultant plasmid was electroporated into electrocompetent Mycobacterium B3683, and kanamycin-resistant transformants were selected on L-agar containing 50 μg/ml kanamycin.

After confirmation of the correct cointegration into the chromosome by Southern hybridization, two independent clones were grown without kanamycin selection and then plated onto L-agar containing 5% sucrose to select sucrose-resistant, kanamycin-sensitive clones. As these arose by recombinational resolution of a gene duplication in the chromosome, they could have resulted from a replacement of the chromosomal ksdA gene (SEQ ID NO:1) with the targeted deletion or reintroduction of the wild-type sequences. Eighty clones were tested for conversion of AD to ADD, and 75% were found to be unable to carry out this conversion. Confirmation of the ksdA (SEQ ID NO:1) deletion was carried out by PCR and Southern hybridization.

### 8) Determination and Deletion of Gene Responsible for Cholesterol to X1/X2 Conversion

Since the transposon insertions that reduced X1/X2 conversion from cholesterol were found within a four gene operon, it was necessary to construct multiple deletions to determine polar effects on downstream expression. As limited flanking sequence was available for constructing a deletion in cxgA (SEQ ID NO:8), we constructed individual deletions in cxgB (SEQ ID NO:9), cxgC (SEQ ID NO:10) and cxgD (SEQ ID NO:11), as well as all three combined. Deletions were carried out using a method similar to that described in the section above. From the analysis of these deletions, it was determined that cxgB (SEQ ID NO:9) was required for the conversion of cholesterol to compounds X1 and X2. In addition, it was determined that cxgD (SEQ ID NO:11) encoded a likely negative regulator of the expression of the operon, as its deletion resulted in a higher rate of X1 and X2 production than the wild-type strain. Deletion of cxgC (SEQ ID NO:10) had no effect on production of X1 or X2. The combined deletion of cxgB (SEQ ID NO:9), cxgC (SEQ ID NO:10) and cxgD (SEQ ID NO:11) resulted in the loss of X1 and X2 production. The first gene in the operon, cxgA (SEQ ID NO:8), may also be required for the conversion of cholesterol to X1 and X2.

Because CxgB (SEQ ID NO:13) and possibly, CxgA (SEQ ID NO:12) are actively involved in the production of compounds X1 and X2, these genes can be overexpressed or modified to improve X1 and X2 production. Additionally, elimination of the cxgD gene (SEQ ID NO:11) would have a similar effect.

### 9) Generation of Combined Deletion Mutant

Because the method used to generate the individual deletions does not result in the introduction of an antibiotic-resistance marker, the combination of both mutations, resulting in loss of ADD and X1/X2 production, was carried out by serial deletion of each; starting with the ksdA deletion (SEQ ID NO:8), followed by deleting cxgB (SEQ ID NO:9). The final strain was confirmed by Southern hybridization and the cholesterol conversion phenotype was determined in a shake-flask assay.

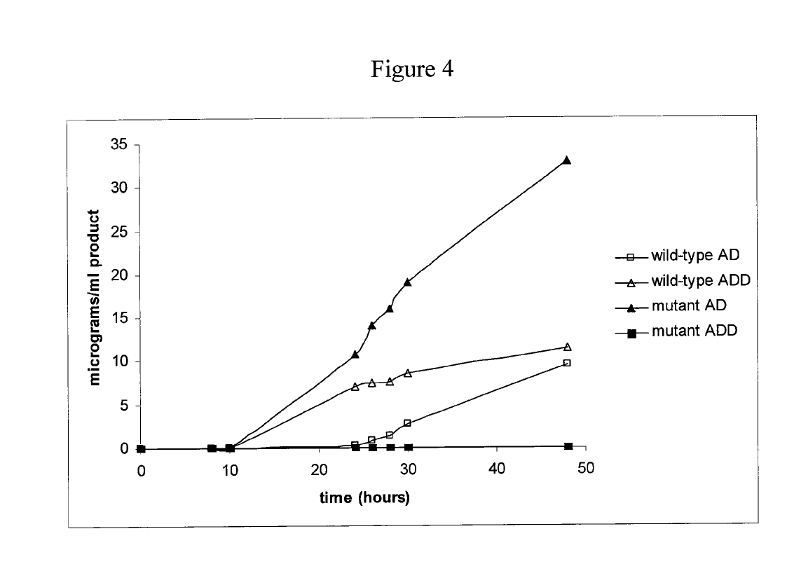

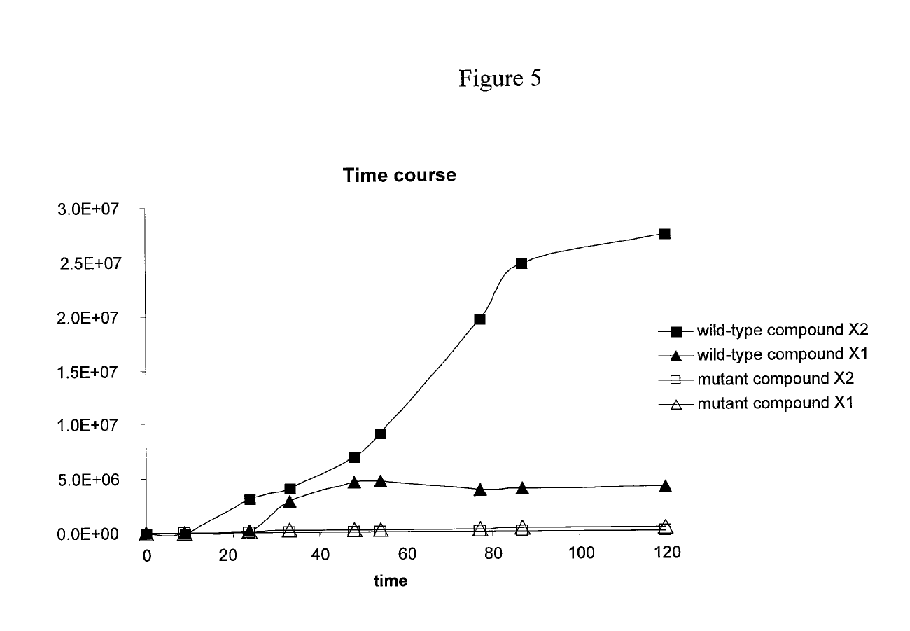

As shown in FIG. 4 and FIG. 5, the final mutant produced no detectable levels of AD and very low levels of X1 and X2. Slightly higher levels of testosterone were produced by this double deletion mutant as compared to the wild-type strain. FIG. 4 graphically illustrates data showing a time course for conversion of cholesterol to AD and ADD by wild-type and ΔksdA/ΔcxgB mutant. FIG. 5 graphically illustrates data showing a time course for conversion of cholesterol to Compound X1 and X2 by wild-type and ΔksdA/ΔcxgB mutant. For FIG. 4 and FIG. 5: Y-axis values represent LC/MS/MS peak area responses and not absolute quantitation of product.

### 10) Analysis of Samples at Pilot Plant Scale

The following Mycobacterium strains of the double deletion mutant were cultured at pilot-plant scale in 500 liter fermentors:
Strain 1: Wild-type Mycobacterium ATCC 29472. As noted previously, the sample obtained from the ATCC was streaked onto MYM agar medium and multiple colony morphologies (“morphotypes”) were seen. After characterizing these morphotypes further, it was determined that a strain with a round, wet, yellow phenotype was most amenable to genetic manipulation.
Strain 2: Mycobacterium ADDX. This strain was derived from the wild-type strain and the genes responsible for the production of ADD and Impurity X were removed. This strain produced no detectable level of ADD and very low levels of Impurity X.
Strain 3: Mycobacterium ADDX::Tn1 dry colony variant #8. This strain was derived from Strain 2 by insertion of a transposon, resulting in a dry, spreading colony morphology. It also produced no ADD and very low levels of Impurity X.
Strain 4: Mycobacterium ADDX::Tn1 dry colony variant #2. Like strain 3, this strain was derived from Strain 2 by insertion of a transposon, resulting in a dry, spreading colony morphology. It had slightly different morphology than strain 3 but also similar produced no ADD and very low levels of Impurity X.
Strain 5: Mycobacterium ADDX::Tn3. This strain was also derived from Strain 2 by insertion of a transposon but had the same round, wet, yellow phenotype of its parent. It appeared to produce significantly more AD than Strain 2.

Three independent methods were used to evaluate the composition of the samples, LC/MS/MS, GC/FID and NMR, as follows:

a) LC/MS/MS

This method was used with available standards AD, ADD, testosterone, and compounds X1 and X2. Phytosterols were not included in the analysis.

The results indicated that no detectable levels of ADD or X2 were present. Although trace amounts of X1 were present in the crude preparations, none could be detected in the crystallized samples. With the exception of one batch, less than 0.5% testosterone was found in the samples.

b) GC/FID

This method was developed to detect as many compounds as possible in the samples, including substrate phytosterols. It was clear that the crude samples contained additional unidentified components. Very little, if any, substrate phytosterols can be seen. Again, no ADD or X2 could be detected and only trace amounts of X1 were present in the crude samples.

With the exception of one batch, all samples contained <0.3% testosterone. Any discrepancy in the testosterone levels of the crystallized samples from the LC/MS/MS data may be accounted for by the fact that all detectable compounds are included in the %-calculation by this method, in contrast to LC/MS/MS. Alternatively, the discrepancy could also result from the limited separation of testosterone from AD in this method and the difficulty in accurately integrating the specific peak area. In regards to “other” compounds, these were not identifiable from the available standards. In the crystallized samples, although the total level of “others” was 1% and 1.2%, the highest level of any single species was 0.3-0.4%.

c) NMR

This method was primarily to confirm the previous methods and was limited to the analysis of AD, ADD and testosterone levels. As in the previous methods, no ADD was detected. Testosterone levels were 0.4-0.5%, depending on where peak integration points were set.

Assays

1) Microtiter Assay for AD to ADD Conversion

Clones to be tested for AD to ADD conversion were inoculated from colonies into 200 μl of 2×YT media in 96-well microtiter plates and incubated for 24 hours at 30° C. in a HIGRO™ incubator (400 rpm) with intermittent aeration. A 20 μl aliquot of AD (100 μM in 2×YT) was added (final concentration of 10 μM AD), and the cultures were incubated for an additional 16 to 18 hours. Conversion reactions were terminated by mixing the entire culture volume of each well with 800 μl acetonitrile in a corresponding well of a polypropylene 96-deep-well microtiter dish. After centrifugation to remove cell debris, a 100 μl aliquot was removed and transferred to another 96-well microtiter dish for LC/MS/MS analysis (see below).

2) Microtiter Assay for Cholesterol Conversion

Clones to be tested for analysis of cholesterol conversion were grown essentially as described above. A 20 μl aliquot of cholesterol-glucose solution (prepared by adding 1/10 volume of 100 mg/ml cholesterol suspension in 5% Tween-20 to 40% glucose) was added to the cells for a final concentration of 1 mg/ml cholesterol, 0.05% Tween-20 and 4% glucose. After an additional incubation of 16 to 18 hours at 30° C., the conversion reactions were stopped by addition of the volume of each well to 800 μl acetonitrile in a 96-deep-well microtiter plate. After centrifugation to remove cell debris, a 100 μl aliquot was transferred for analysis by LC/MS/MS (see below).

3) Shake Flask Assay for Cholesterol Conversion

A single colony of the strain to be tested was grown overnight in 25 ml of 2×YT in a 250 ml flask at 220 rpm and 30° C. After an OD₆₀₀ of 0.2-0.3 was obtained, 5 ml of the culture was transferred to 50 ml of fresh 2×YT medium containing 5 mg/ml cholesterol and 0.25% Tween-20. Then 100 μl of culture were sampled at various time points and added to 900 μl of acetonitrile in a 96-deep-well plate to stop the conversion and to extract the products. After the completion of the experiment, the plate was centrifuged for 5 minutes to remove cell debris and 100 μl of the supernatant was analyzed by LC/MS/MS (see below).

4) LC/MS/MS Analysis for Conversion Products

LC/MS/MS conditions for analysis were as follows: samples were injected from 96-well plates using a CTCPAL™ (CTCPal) auto-sampler (LEAP Technologies, Carrboro, N.C.) into an isocratic mixture of water/acetonitrile (0.1% formic acid) at 45/55. This mixture was provided by LC-10ADVP™ (LC-10ADvp) pumps (Shimadzu, Kyoto, Japan) at 1.0 ml/min through a SYNERGI MAXRP™ (Phenomenex, Torrance Calif.) 50×2 mm column and into the API4000 TURBOION-SPRAY™ triple-quad mass spectrometer (Applied Biosystems, Foster City, Calif.). Ion spray and MRM (multiple reaction monitoring) were performed for the analytes of interest in the positive ion mode, and each analysis lasted 1.2 minutes.

The following parent/fragment ion combinations were used to monitor the compounds of interest: androstenedione, 287.26/97.85; androstadienedione, 285.23/121.65; testosterone, 289.21/97.75; 21-hydroxy-20-methylpregna-1,4-diene-3-one, 329.30/121.42; 21-hydroxy-20-methylpregn-4-en-3-one, 331.30/109.45.

Androstenedione, androstadienedione, testosterone and standards were purchased from Sigma Chemicals (St. Louis, Mo.). 21-hydroxy-20-methylpregna-1,4-diene-3-one (Compound X2) was purchased from Fisher Scientific (Pittsburgh, Pa.). 21-hydroxy-20-methylpregn-4-en-3-one (Compound X1) was prepared by extraction of a large-scale cholesterol conversion using the ksdA Tn5 mutant, which is unable to produce compound X2 due to the defect in the 3-ketosteroid-Δ1-dehydrogenase. Flash chromatography was used to purify compound X1, and its identity was confirmed by NMR.

5) Southern Hybridization for Confirmation of Mutants

Strains to be tested were grown to saturation in 2×YT, and 1 ml of culture was used to prepare chromosomal DNA using the EPICENTRE™ genomic DNA purification kit (Epicentre, Madison, Wis.). DNA was digested with appropriate restriction enzymes, separated by agarose gel electrophoresis, transferred to a nylon filter and hybridized with a ³²P-radiolabeled PCR product from the corresponding region flanking the deletion. Autoradiography was used to determine the size of the hybridizing chromosomal fragment to verify the expected deletions.

| (SEQ ID NO: 1) | |
|---|---|
| Gene sequence of ksdA | |
| (SEQ ID NO: 1) | |
| ATGACTGAACAGGACTACAGTGTCTTTGACGTAGTGGTGGTAGGGAGCGGTGCTGCCGGCA | |
| TGGTCGCCGCCCTCACCGCCGCTCACCAGGGACTCTCGACAGTAGTCGTTGAGAAGGCTCC | |
| GCACTATGGCGGTTCCACGGCGCGATCCGGCGGCGGCGTGTGGATTCCGAACAACGAGGTT | |
| CTGCAGCGTGACGGGGTCAAGGACACCCCCGCCGAGGCACGCAAATACCTGCACGCCATCA | |
| TCGGCGATGTGGTGCCGGCCGAGAAGATCGACACCTACCTGGACCGCAGTCCGGAGATGTT | |
| GTCGTTCGTGCTGAAGAACTCGCCGCTGAAGCTGTGCTGGGTTCCCGGCTACTCCGACTAC | |
| TACCCGGAGACGCCGGGCGGTAAGGCCACCGGCCGCTCGGTCGAGCCCAAGCCGTTCAATG | |
| CCAAGAAGCTCGGTCCCGACGAGAAGGGCCTCGAACCGCCGTACGGCAAGGTGCCGCTGAA | |
| CATGGTGGTGCTGCAACAGGACTATGTCCGGCTCAACCAGCTCAAGCGTCACCCGCGCGGC | |
| GTGCTGCGCAGCATCAAGGTGGGTGTGCGGTCGGTGTGGGCCAACGCCACCGGCAAGAACC | |
| TGGTCGGTATGGGCCGGGCGCTGATCGCGCCGCTGCGCATCGGCCTGCAGAAGGCCGGGGT | |
| GCCGGTGCTGTTGAACACCGCGCTGACCGACCTGTACCTCGAGGACGGGGTGGTGCGCGGA | |
| ATCTACGTTCGCGAGGCCGGCGCCCCCGAGTCTGCCGAGCCGAAGCTGATCCGAGCCCGCA | |
| AGGGCGTGATCCTCGGTTCCGGTGGCTTCGAGCACAACCAGGAGATGCGCACCAAGTATCA | |
| GCGCCAGCCCATCACCACCGAGTGGACCGTCGGCGCAGTGGCCAACACCGGTGACGGCATC | |
| GTGGCGGCCGAAAAGCTCGGTGCGGCATTGGAGCTCATGGAGGACGCGTGGTGGGGACCGA | |
| CCGTCCCGCTGGTGGGCGCCCCGTGGTTCGCCCTCTCCGAGCGGAACTCCCCCGGGTCGAT | |
| CATCGTCAACATGAACGGCAAGCGGTTCATGAACGAATCGATGCCCTATGTGGAGGCCTGC | |
| CACCACATGTACGGCGGTCAGTACGGCCAAGGTGCCGGGCCTGGCGAGAACGTCCCGGCAT | |
| GGATGGTCTTCGACCAGCAGTACCGTGATCGCTATATCTTCGCGGGATTGCAGCCCGGACA | |
| ACGCATCCCGAAGAAATGGATGGAATCGGGCGTCATCGTCAAGGCCGACAGCGTGGCCGAG | |
| CTCGCCGAGAAGACCGGTCTTGCCCCCGACGCGCTGACGGCCACCATCGAACGGTTCAACG | |
| GTTTCGCACGTTCCGGCGTGGACGAGGACTTCCACCGTGGCGAGAGCGCCTACGACCGCTA | |
| CTACGGTGATCCGACCAACAAGCCGAACCCGAACCTCGGCGAGATCAAGAACGGTCCGTTC | |
| TACGCCGCGAAGATGGTACCCGGCGACCTGGGCACCAAGGGTGGCATCCGCACCGACGTGC | |
| ACGGCCGTGCGTTGCGCGACGACAACTCGGTGATCGAAGGCCTCTATGCGGCAGGCAATGT | |
| CAGCTCACCGGTGATGGGGCACACCTATCCCGGCCCGGGTGGCACAATCGGCCCCGCCATG | |
| ACGTTCGGCTACCTCGCCGCGTTGCATCTCGCTGGAAAGGCCTGA | |
| (SEQ ID NO: 2) | |
| protein sequence of KsdA | |
| (SEQ ID NO: 2) | |
| MTEQDYSVFDVVVVGSGAAGMVAALTAAHQGLSTVVVEKAPHYGGSTARSGGGVWIPNNEV | |
| LQRDGVKDTPAEARKYLHAIIGDVVPAEKIDTYLDRSPEMLSFVLKNSPLKLCWVPGYSDY | |
| YPETPGGKATGRSVEPKPFNAKKLGPDEKGLEPPYGKVPLNMVVLQQDYVRLNQLKRHPRG | |
| VLRSIKVGVRSVWANATGKNLVGMGRALIAPLRIGLQKAGVPVLLNTALTDLYLEDGVVRG | |
| IYVREAGAPESAEPKLIRARKGVILGSGGFEHNQEMRTKYQRQPITTEWTVGAVANTGDGI | |
| VAAEKLGAALELMEDAWWGPTVPLVGAPWFALSERNSPGSIIVNMNGKRFMNESMPYVEAC | |
| HHMYGGQYGQGAGPGENVPAWMVFDQQYRDRYIFAGLQPGQRIPKKWMESGVIVKADSVAE | |
| LAEKTGLAPDALTATIERFNGFARSGVDEDFHRGESAYDRYYGDPTNKPNPNLGEIKNGPF | |
| YAAKMVPGDLGTKGGIRTDVHGRALRDDNSVIEGLYAAGNVSSPVMGHTYPGPGGTIGPAM | |
| TFGYLAALHLAGKA | |
| Alignment of Mycobacterium B3683 KsdA and homologs | |
| (SEQ ID NO: 1) | |
| B3683 = Mycobacterium B3683 3-ketosteroid-Δ1- | |
| dehydrogenase | |
| (SEQ ID NO: 3) | |
| MAP = Mycobacterium avium paratuberculosis MAP0530c | |
| (SEQ ID NO: 4) | |
| MT = Mycobacterium tuberculosis putative 3- | |
| ketosteroid-Δ1-dehydrogenase | |
| (SEQ ID NO: 5) | |
| NF = Nocardia farcinica putative 3-ketosteroid-Δ1- | |
| dehydrogenase | |
| (SEQ ID NO: 6) | |
| SA = Streptomyces avermitilis putative 3- | |
| ketosteroid-Δ1-dehydrogenase | |
| (SEQ ID NO: 7) | |
| RE = Rhodococcus erythropolis 3-ketosteroid-Δ1- | |
| dehydrogenase | |
| (SEQ ID NO: 8) | |
| CT = Comomonas testosteroni 3-ketosteroid-Δ1- | |
| dehydrogenase | |
| | 1 50 |
| B3683 | ........MT EQDYSVFDVV VVGSGAAGMV AALTAAHQGL STVVVEKAPH |
| MAP | ........MF YMSAQEYDVV VVGSGGAGMV AALTAAHRGL STIVIEKAPH |
| MT | ........MF YMTVQEFDVV VVGSGAAGMV AALVAAHRGL STVVVEKAPH |
| NF | ......MTDP VLDPHSYDVV VVGSGAAGMT AALTAAHHGL RVVVLEKAAH |
| SA | .......... .......... ........MT AALTAAKQGL SCVVVEKAAT |
| RE | MAKNQAPPAT QAKDIVVDLL VIGSG.TGMA AALTANELGL STLIVEKTQY |
| CT | .......... .MAEQEYDLI VVGSGAGAML GAIRAQEQGL KTLVVEKTEL |
| | 51 100 |
| B3683 | YGGSTARSGG GVWIPNNEVL QRDGVKDTPA EARKYLHAII GDVVPAEKID |
| MAP | FGGSTARSGG GVWIPNNEVL KRDGVKDTPE AARTYLHGII GDVVEPERID |
| MT | YGGSTARSGG GVWIPNNEVL KRRGVRDTPE AARTYLHGIV GEIVEPERID |
| NF | YGGSTARSGG GVWIPGNKAL RASGRPDDRE EARTYLHSII GDVVPKERID |
| SA | FGGSAARSGA GIWIPNNPVI LAAGVPDTPA KAAAYLAAVV GPDVSADRQR |
| RE | VGGSTARSGG AFWMPANPIL AKAGAGDTVE RAKTYVRSVV GDTAPAQRGE |
| CT | FGGTSALSGG GIWIPLNYDQ KTAGIKDDLE TAFGYMKRCV RGMATDDRVL |
| | 101 150 |
| B3683 | TYLDRSPEML SFVLKNSPLK LCWVPGYSDY YPETPGGKAT GRSVEPKPFN |
| MAP | TYLERGPEML SFVLKHTPLK MCWVPRYSDY YPESPGGRAE GRSIEPKPFN |
| MT | AYLDRGPEML SFVLKHTPLK MCWVPGYSDY YPEAPGGRPG GRSIEPKPFN |
| NF | TYIDRGAEAF DFVLDHTPLQ MKWVPGYSDY YPEAPGGRGE GRSCEPKPFD |
| SA | AFLGHGPAMI SFVMANSPLR FRWMEGYSDY YPELSGGLPN GRSIEPDQLD |
| RE | AFVDNGAATV DMLYRTTPMK FFWAKEYSDY HPELPGGSAA GRTCECLPFD |
| CT | AYVETASKMA EYLRQIG.IP YRAMAKYADY YPHIEGSRPG GRTMDPVDFN |
| | 151 200 |
| B3683 | AKKLGPDEKG LE....PPYG KVPLNMVVLQ QDYVRLNQLK RHP.RGVLRS |
| MAP | ARKLGPDEAG LE....PAYG KVPLNVVVMQ QDYVRLNQLK RHP.RGVLRS |
| MT | ARKLGADMAG LE....PAYG KVPLNVVVMQ QDYVRLNQLK RHP.RGVLRS |
| NF | LKVLGPEKDK LE....PAYA KAPLNVVVMQ ADFVRLNLIR RHP.KGMLRA |
| SA | GNILGAELAH LN....PSYM AVPAGMVVFS ADYKWLTLSA VSA.KGLAVA |
| RE | ASVLGAERGR LR....PGLM EAGLPMPVTG ADYKWMNLMV KKPSKAFPRI |
| CT | AARLGLAALE TMRPGPPGNQ LFGRMSISAF EAHSMLSREL KSRFTILGIM |
| | 201 250 |
| B3683 | IKVGVRSVWA NATGK.NLVG MGRALIAPLR IGLQKAGVPV LLNTALTDLY |
| MAP | LKVGARTMWA KATGK.NLVG MGRALIGPLR IGLQRAGVPV VLNTALTDLY |
| MT | MKVGARTMWA KATGK.NLVG MGRALIGPLR IGLQRAGVPV ELNTAFTDLF |
| NF | MRVGARTYWA KFTGK.HIVG MGQAIIAAMR KGLMDANVPL LLNTPMTKLV |
| SA | AECLARGTKA ALLGQ.KPLT MGQSLAAGLR AGLLAAQVPV WLNTPLTDLY |
| RE | IRRLAQGVYG KYVLKREYIA GGQALAAGLF AGVVQAGIPV WTETSLVRLI |
| CT | LKYFLDYPWR NKTRRDRRMT GGQALVAGLL TAANKVGVEM WHNSPLKELV |
| | 251 300 |
| B3683 | LED.GVVRGI YVREAGAPES AEPKLIRARK GVILGSGGFE HNQEMRTKYQ |
| MAP | LED.GVVRGV YVRDSQAAES AEPRLIRARR GVILASGGFE HNEQMRVKYQ |
| MT | VEN.GVVSGV YVRDSHEAES AEPQLIRARR GVILACGGFE HNEQMRIKYQ |
| NF | VED.GRVTGV EALHE..... GEPVVFSARY GVVLGSGGFE HNAEMRAKYQ |
| SA | REN.GTVTGA VVAKG..... GSAGLVRARH GVVVGSGGFE HNAAMRDQYQ |
| RE | TED.GRVTGA VVVQD..... GREVTVTARR GVVLAAGGFD HNMEWRHKYQ |
| CT | QDASGRVTGV IVERN..... GQRQQINARR GVLLGAGGFE RNQEMRDQYL |
| | 301 350 |
| B3683 | RQPITTEWTV G.AVANTGDG IVAAEKLGAA LELMEDAWWG PTVPLV.GAP |
| MAP | RAPITTEWTV G.AKANTGDG ILAAEKLGAA LELMEDAWWG PTVPLV.GAP |
| MT | RAPITTEWTV G.ASANTGDG ILAAEKLGAA LDLMDDAWWG PTVPLV.GKP |
| NF | RQPITTEWTT G.AAANTGDG IRAGMEIGAD VDFMEDAWWG PTIFKG.GRP |
| SA | RQPIGTAWTV G.AKENTGDG IRAGERAGAA LDLMDDAWWG PTIPLP.DQP |
| RE | SESLGEHESL G.AEGNTGEA IEAAQELGAG IGSMDQSWWF PAVASIKGRP |
| CT | NKPSKAEWTA TPVGGNTGDA HRAGQAVGAQ LALMDWSWGV PTMDVPKEPA |
| | 351 400 |
| B3683 | .WFALSERNS PGSIIVNMNG KRFMNESMPY VEACHHMYGG QYGQGAGPGE |
| MAP | .WFALSERNS PGSIIVNMSG KRFMNESMPY VEACHHMYGG EFGQGPGPGE |
| MT | .WFALSERNS PGSIIVNMSG KRFMNESMPY VEACHHMYGG EHGQGPGPGE |
| NF | .WFALAERNL PGCVIVNAQG KRFANESAPY VEAVHAMYGG EYGQGEGPGE |
| SA | .YFCLAERTL PGGLLVNAAG ARFVNEAAPY SDVVHTMYER NP...TAP.. |
| RE | PMVMLAERAL PGSFIVDQTG RRFVNEATDY MSFGQRVLER EK...AGDP. |
| CT | FRGIFVERSL PGCMVVNSRG QRFLNESGPY PEFQQAMLAE HAK...GNG. |
| | 401 450 |
| B3683 | NVPAWMVFDQ QYRDRYIFAG .LQPGQRIPK KWMES....G VIVKADSVAE |
| MAP | NIPAWLVFDQ QYRDRYIFAG .LQPGQRIPR KWLES....G VIIQADTLEE |
| MT | NIPAWLVFDQ RYRDRYIFAG .LQPGQRIPS RWLDS....G VIVQADTLAE |
| NF | NIPAWLVFDQ RYRNRYIFAG .LQPGQRFPS RWMED....Q NIVKADTLAE |
| SA | DIPAWLIVDQ NYRNRYLFKD .VAPTLAFPG SWYDS....G AAHKAWTLDA |
| RE | AESMWFVFDQ EYRNSYVFAG GIFPRQPLPQ AFFES....G IAHQASSPAE |
| CT | GVPAWIVFDA SFRAQNPMGP .LMPGSAVPD SKVRKSWLNN VYWKGETLED |
| | 451 500 |
| B3683 | LAEKTGLAPD ALTATIERFN GFARSGVDED FHRGESAYDR YYGDPTNKPN |
| MAP | LASRAGLPVD EFLATVQRFN GFARTGIDED YHRGESAYDR YYGDPTNKPN |
| MT | LAGKAGLPAD ELTATVQRFN AFARSGVDED YHRGESAYDR YYGDPSNKPN |
| NF | LAELIGVPVG NLTATVERFN KFAETGKDED FGRGDSHYDR YYGDPTVKPN |
| SA | LAGRIGMPAA ALRATVNRFN SLALSGDDTD FQRGDSTYDH YYTDPAIVPN |
| RE | LARKVGLPED AFAESFQKFN EAAAAGSDAE FGRGGSAYDR YYGDPTVSPN |
| CT | LARQIGVDAT GLQDSARRMT EYARAGKDLD FDRGGNVFDR YYGDPRLK.N |
| | 501 550 |
| B3683 | PNLGEIKNGP FYAAKMVPGD LGTKGGIRTD VHGRALRDDN SVIEGLYAAG |
| MAP | PNLGEISHPP YYAAKMVPGD LGTKGGIRTD IHGRALRDDG SIIEGLYAAG |
| MT | PNLGEVGHPP YYGAKMVPGD LGTKGGIRTD VNGRALRDDG SIIDGLYAAG |
| NF | PCLAALVQGP FYAAKIVPGD LGTKGGLVAD ESGRVLREDG SPIPGLYASG |
| SA | SCLAPLWLAP YYAFKIVPGD LGTKGGLRTD ARARVLRADG SVIPGLYAAG |
| RE | PNLRQLDKSA LYAVKMTLSD LGTCGGVQAD ENARVLREDG SVIDGLYAIG |
| CT | PNLGPIEKGP FYAMRLWPGE IGTKGGLLTD REGRVLDTQG RIIEGLYCVG |
| | 551 |
| B3683 | NVSSPVMGHT YPGPGGTIGP AMTFGYLAAL HLAGKA (563) |
| MAP | NVSAPVMGHT YPGPGGTIGP AMTFGYLAAL HIAGEN (563) |
| MT | NVSAPVMGHT YPGPGGTIGP AMTFGYLAAL HIADQAGKR (566) |
| NF | NCSTPVMGHT YAGPGATIGP AITFGYLSVL DILARKNEQS PAASGTA (571) |
| SA | NASAAVMGHS YAGAGSTIGP AMTFGYIAAL DIAAAAGS (535) |
| RE | NTAANAFGHT YPGAGATIGQ GLVYGYIAAH HAAEK (565) |
| CT | NNSASVMGPA YAGAGSTLGP AMTFAFRAVA DMLGKPLPIE NPHLLGKTV (576) |
| | IDENTITY/SIMILARITY TO B3683 |
| MAP | 83/92% |
| MT | 80/90% |
| NF | 65/76% |
| SA | 51/62% |
| RE | 42/59% |
| CT | 38/55% |
| (SEQ ID NO: 9) | |
| Gene sequence cxgA gene | |
| (SEQ ID NO: 9) | |
| TTGGGTTTGCGTGGTGACGCAGCGATCGTCGGGTTTCACGAGCTACCTGCGACGCGGAAGCCGA | |
| CCGGGACCGCGGAGTTCACCATCGAACAGTGGGCGCGGTTGGCGGCCGCGGCGGTGGCCGACGC | |
| GGGGCTGTCGGTCCAGCAGGTCGACGGGCTGGTGACCTGCGGGGTCATGGAGTCCCAGCTGTTC | |
| GTCCCCTCCACAGTCGCCGAGTATCTGGGTCTGGCGGTCAATTTCGCCGAGATCGTCGATCTCG | |
| GCGGCGCCTCGGGCGCGGCCATGGTGTGGCGCGCGGCGGCGGCGATCGAACTGGGGCTCTGCCA | |
| GGCGGTGCTGTGCGCCATCCCAGCCAACTACCTGACCCCGATGTCGGCGGAGCGTCCCTACGAT | |
| CCCGGCGACGCGCTGTACTACGGGGCGTCCAGCTTCCGGTACGGCTCGCCGCAGGCCGAGTTCG | |
| AGATTCCCTACGGCTACCTCGGACAGAACGGTCCGTACGCGCAGGTCGCCCAGATGTACTCGGC | |
| CGCATACGGATACGACGAGACCGCGATGGCCAAGATCGTCGTCGACCAGCGGGTGAACGCCAAC | |
| CACACACCCGGGGCGGTGTTCCGGGACAAACCGGTGACCATCGCCGATGTCCTGGACAGCCCGA | |
| TCATCGCGTCTCCGCTGCACATGCTGGAAATCGTCATGCCGTGCATGGGGGGATCGGCAGTGCT | |
| CGTCACCAATGCCGAACTGGCCCGCGCCGGCCGCCACCGACCGGTCTGGATCAAGGGGTTCGGC | |
| GAACGGGTGCCCTACAAGTCCCCGGTCTATGCCGCCGATCCGCTCCAGACACCGATGGTGAAGG | |
| TCGCCGAATCCGCCTTCGGGATGGCCGGCCTGACCCCGGCCGACATGGACATGGTGTCGATCTA | |
| CGACTGCTACACCATCACCGCCCTGCTGACGTTGGAGGACGCGGGTTTCTGTGCCAAGGGCACG | |
| GGAATGCGGTTCGTCACCGACCACGACCTGACCTTCCGCGGTGACTTCCCGATGAACACCGCAG | |
| GCGGACAGCTCGGCTACGGCCAGCCCGGCAATGCCGGTGGCATGCACCATGTGTGCGATGCCAC | |
| CCGGCAGCTGATGGGACGCGCCGGGGCAACCCAGGTCGCGGACTGTCACCGCGCCTTCGTCTCG | |
| GGCAACGGTGGCGTGCTCAGCGAACAAGAAGCTCTCGTCCTGGAGGGGGAT | |
| (SEQ ID NO: 10) | |
| protein sequence of CxgA | |
| MGLRGDAAIVGFHELPATRKPTGTAEFTIEQWARLAAAAVADAGLSVQQVDGLVTCGVMESQLF | |
| VPSTVAEYLGLAVNFAEIVDLGGASGAAMVWRAAAAIELGLCQAVLCAIPANYLTPMSAERPYD | |
| PGDALYYGASSFRYGSPQAEFEIPYGYLGQNGPYAQVAQMYSAAYGYDETAMAKIVVDQRVNAN | |
| HTPGAVERDKPVTIADVLDSPIIASPLHMLEIVMPCMGGSAVLVTNAELARAGRHRPVWIKGFG | |
| ERVPYKSPVYAADPLQTPMVKVAESAFGMAGLTPADMDMVSIYDCYTITALLTLEDAGFCAKGT | |
| GMRFVTDHDLTFRGDFPMNTAGGQLGYGQPGNAGGMHHVCDATRQLMGRAGATQVADCHRAFVS | |
| GNGGVLSEQEALVLEGD | |
| Alignment of Mycobacterium B3683 CxgA and homologs | |
| (SEQ ID NO: 11) | |
| B3683 = Mycobacterium B3683 CxgA | |
| (SEQ ID NO: 12) | |
| MAP1 = Mycobacterium avium paratuberculosis MAP4302c | |
| (SEQ ID NO: 13) | |
| MAP = Mycobacterium avium paratuberculosis MAP1462 | |
| (SEQ ID NO: 14) | |
| PSP = Polaromonas sp. acetyl CoA acetylatransferase | |
| (SEQ ID NO: 15) | |
| RE = Ralstonia eutropha acetyl CoA acetylatransferase | |
| (SEQ ID NO: 16) | |
| RP = Rhodopseudomonas palustris putative thiolase | |
| | 1 50 |
| B3683 | .......... .......LGL RGDAAIVGFH ELP.ATRKPT GTAEFTIEQW |
| MAP1 | .......... .......MGL RGEAAIVGYV ELPPERLSKA SPAPFVLEQW |
| MAP2 | .......... ......MTGL RGEAAIVGIA ELP.AERRPT GPPRFTLDQY |
| PSP | .......... .......... ....MIVGVA DLPLKDGK.V LRPMSVLEAQ |
| RE | .......... .......MTL NGSAYIVGAY EHPTRK.... ADDLSVARLH |
| RP | MDSGLAPRGA PRNDERDGVC NRQAAIMSYI TGVGLTRFGK IDGSTTLSLM |
| | 51 100 |
| B3683 | ARLAAAAVAD AGLSVQQVDG LVTCG...VM ESQLFVPSTV AEYLGLAVNF |
| MAP1 | AEPGAAALQD AGLPGEVVNG IVASH...LA ESEIFVPSTI AEYLGVGARF |
| MAP2 | ALLAKLVIED AGVDPGRVNG LLTHG...VA ESAMFAPATL CEYLGLACDF |
| PSP | ALVARDALKD AGIPMSEVDG LLTAGLWGVP GPGQLPTVTL SEYLGITPRF |
| RE | ADVARGALAD AGLTAADVDG YFCAG..DAP GLG...TTTI VEYLGLKPRH |
| RP | REAAEAAIAD AGLKRGDIDG LLCGYS..TT MPHIMLATVF AEHFGILPSH |
| | 101 150 |
| B3683 | AEIVDLGGAS GAAMVWRAAA AIELGLCQAV LCAIPANYLT PMSAERPYDP |
| MAP1 | AEHVVLGGAS AAAMVWRAAA AIELGICDAV LCALPARYIT PSSKKKPRPM |
| MAP2 | GERVDLGGAS SAGMVWRAAA AVELGICEAA LAVVPGSASV PHSARRP..P |
| PSP | IDSTNIGGSA FEAHVAHAAM AIEAGRCEVA LITYGSLQ.. .......... |
| RE | VDSTECGGSA PILHVAHAAE AIAAGRCNVA LITLAGRPRA .......... |
| RP | CHAVQVGGAT GMAMAMLAYQ LVESGAAKNI LVVGGENRLT G......... |
| | 151 200 |
| B3683 | GDALYYGASS FRYGSPQAEF EIPYGYLGQN GPYAQVAQMY SAAYGYDETA |
| MAP1 | VDAMFFGSSS NQYGSPQAEF EIPYGNLGQN GPYGQVAQRY AAVYGYDERA |
| MAP2 | PESNWYGASS NNYGSPQAEF EIPYGNVGQN APYAQIAQRY AAEFGYDPAA |
| PSP | .KSEMSRNLA GRPAVLTMQY ETPWGMPTPV GGYAMAAKRH MHEYGTTSEQ |
| RE | .AGAALALRA PDPDAPDVAF ELPFGPATQN .LYGMVAKRH MYEFGTTSEQ |
| RP | ..QSRDASVQ ALAQVGHPIY EVPLGPTIPA .YYGLVASRY MHDHGVTEED |
| | 201 250 |
| B3683 | MAKIVVDQRV NANHTPGAVF RDKPVTIADV LDSPIIASPL HMLEIVMPCM |
| MAP1 | MAKIVVDQRV NANHTDGAIW RDTPLTVEDV LASPVIADPL HMLEIVMPCV |
| MAP2 | LAKIAVDQRT NACAHPGAVF FGTPITAADV LDSPMIADPI HMLETVMRVH |
| PSP | LAEIAVATRQ WAALNPAATM RD.PLSIEDV LKSPMVCDPM HLLDICLVTD |
| RE | LAWIKVAASH HAQHNPHAML RN.VVTVEDV VNSPMVADPL HRLDCCVMSD |
| RP | LAEFAVLMRS HAITHPGAQF HE.PISVAEV MASKPIASPL KLLDCCPVSD |
| | 251 300 |
| B3683 | GGSAVLVTNA ELARAGRHRP VWIKGFGERV PYKSPVYAAD .PLQTPMVKV |
| MAP1 | GGAAVVVANA DLAKRARHRP VWVKGFGEHV PFKTPTYAED .LLRTPIAAA |
| MAP2 | GGAAVLIANA DLARRGRHRP VWIKGFGEHI AFKTPTYAED .LLSTPIARA |
| PSP | GGGAVVMTTA EHARALGRKA VHVRGYGESH THWTIAAMPD LARLTAAEVA |
| RE | GGGALIVARP EIARQLRRPL VKVRGTGEAP KHAMGGNID. .LTWSAAAWS |
| RP | GGAALVIS.. .RE.PTTAHQ IKVRGCGQAH THQHVTAMP. AAGPSGAELS |
| | 301 350 |
| B3683 | AESAFGMAGL TPADMDMVSI YDCYTITALL TLEDAGFCAK GTGMRFVTDH |
| MAP1 | ADTAFAMTGL SRAQMDMVSI YDCYTITVLL SLEDAGFCEK GRGMEFVADH |
| MAP2 | AERAFAMAGL DRPDVDVASI YDCYTITVLM SLEDAGFCAK GQGMQWIGDH |
| PSP | GRDAFAMAGI GHDAIDVVEV YDSFTITVLL TLEALGFCQR GESGAFVSNQ |
| RE | GPAAFAEAGV TPADIKYASL YDSFTITVLM QLEDLGFCKK GEGGKFVADG |
| RP | IARAWATSGV EIADVKYAAV YDSFTITLLM LLEDLGLAAR GEAAARARDG |
| | 351 400 |
| B3683 | .DLTFRGDFP MNTAGGQLGY GQPGNAGGMH HVCDATRQLM GRAGAT.QVA |
| MAP1 | .DLTFRGDFP LNTAGGQLGF GQAGLAGGMH HVCDATRQIM GRAGAA.QVP |
| MAP2 | .DLTHRGDFP LNTAGGQLSF GQAGMAGGMH HVVDGARQIM GRAGDA.QVP |
| PSP | .RTAPGGAFP LNTNGGGLSY AHPGMYG.IF LLIEAVRQLR GECGPR.QIA |
| RE | GLISGVGRLP FNTDGGGLCN NHPANRGGVT KVIEAVRQLR GEAHPAVQVS |
| RP | .YFSRTGAMP LNTHGGLLSY GHCGVGGAMA HLVETHLQMT GRAGDR.QVR |
| | 401 |
| B3683 | DCHRAFVSGN GGVLSEQ... EALVLEGD (401) |
| MAP1 | DCNRAFVSGN GGILSEQ... TTLILEGD (400) |
| MAP2 | GCHTAFVTGN GGIMSEQ... VALLLQGE (402) |
| PSP | NAVTALVHGT GGTLSS...G ATCILSTR (383) |
| RE | NCDLALASGI GGALASRHTA ATLILERE (387) |
| RP | DASLALLHGD GGVLSSH... VSMILERVR (404) |
| | IDENTITY/SIMILARITY TO B3683 |
| MAP1 | 69/81% |
| MAP2 | 63/76% |
| PSP | 34/49% |
| RE | 37/50% |
| RP | 34/46% |
| (SEQ ID NO: 17) | |
| Gene sequence of cxgB | |
| (SEQ ID NO: 17) | |
| ATGACCGAGTCGTCGGCCCGGCCAGTGCCACTGCCCACGCCGACCTCGGCACCGTTCTGGGATG | |
| GCCTGCGCCGGCACGAGGTGTGGGTGCAATTCTCACCGTCATCGGATGCCTACGTGTTCTATCC | |
| GCGCATCCTGGCGCCCGGCACCCTGGCCGATGATCTGTCCTGGCGCCAGATCTCCGGTGATGCC | |
| ACCCTGGTCAGCTTCGCCGTCGCACAGCGACCGGTCGCCCCTCAGTTCGCCGATGCCGTTCCGC | |
| ATCTGCTCGGCGTGGTGCAGTGGACCGAGGGGCCGCGGCTGGCCACCGAGATCGTCGGCGTCGA | |
| TCCGGCTCGACTGCGCATCGGTATGGCCATGACGCCGGTGTTCACCGAACCCGACGGCGCCGAT | |
| ATCACCCTGTTGCACTACACCGCCGCCGAA | |
| (SEQ ID NO: 18) | |
| protein sequence of CxgB | |
| (SEQ ID NO: 18) | |
| MTESSARPVPLPTPTSAPFWDGLRRHEVWVQFSPSSDAYVFYPRILAPGTLADDLSWRQISGDA | |
| TLVSFAVAQRPVAPQFADAVPHLLGVVQWTEGPRLATEIVGVDPARLRIGMAMTPVFTEPDGAD | |
| ITLLHYTAA | |
| Alignment of Mycobacterium B3683 CxgB and homologs | |
| (SEQ ID NO: 18) | |
| B3683 = Mycobacterium B3683 CxgB | |
| (SEQ ID NO: 19) | |
| MAP1 = Mycobacterium avium paratuberculosis MAP4301c | |
| (SEQ ID NO: 20) | |
| RE = Ralstonia eutropha putative nucleic acid binding protein, | |
| Zn finger | |
| (SEQ ID NO: 21) | |
| PSP = Polaromonas sp. putative nucleic acid binding protein, | |
| Zn finger | |
| (SEQ ID NO: 22) | |
| SA = Streptomyces avermitilis hypothetical protein | |
| (SEQ ID NO: 23) | |
| MAP2 = Mycobacterium avium paratuberculosis MAP4296c | |
| | 1 50 |
| B3683 | ....MTESSA RPVPLPTP.T SAPFWDGLRR HEVWVQFSPS SDAYVFYPRI |
| MAP1 | .....MTTFE RPMPVKTP.T TAPFWDALAQ HRIVIQYSPS LQSYVFYPRV |
| RE | .......... ..MAIGHYMD TAAFWAATRE RRLLVQFCTQ TGRWQAYPRP |
| PSP | .......MYD KPLPVIDG.E SRPYWDALKQ HRLTLKRCQD CGKHHFYPRA |
| SA | .....MSGRR FDEPETDA.F TRPYWDAAAE GVLLLRRCAG CGRTHHYPRE |
| MAP2 | MTAEPLRPQT GPVPHASSPL SVPFWEGCRS RQLRYQRCRA CDLANFPPTE |
| | 51 100 |
| B3683 | LAPGTLADDL SWRQISGDAT LVSFAVAQRP VAPQFADAVP HLLGVVQWTE |
| MAP1 | RAPRTLADDL EWREISGMGS LYSYTVAHRP VSPHFADAVP QLLAIVEWDE |
| RE | GSVYTGRRRL AWREVSGDGV LASWTVDR.. MNTPAAADAP RMHAWIDLVE |
| PSP | LCPHCHSDAV EWVDACGTGT IYSYTIARRP AGPAFKADTP YVVAVIDLDE |
| SA | FCPHCWSDDV TWERASGRAT LYTWSVVHRN DLPPFGERTP YVAAVVDLAE |
| MAP2 | HCRQCLSDDI GWQQSGGRGE IYSWTVVHRP VTAEFIP..P NAPAIITLDE |
| | 101 150 |
| B3683 | GPRLATEIVG VDPARLRIGM AMTPVFTEPD GADITLLHYT AAE (138) |
| MAP1 | GPRFSTEMVN VDPAQLRVGM RVQPVFCDYP EHDVTLLRYQ PAD (137) |
| RE | GARILSWLVD CDPARLRVGL AVRVAWISLP DGWQWPAFTI AAHSGGPNGKAP (138) |
| PSP | GARMMTNIVT DDVEAVRIGQ RVT.VQYDDV TEEVTLPKFR LL (133) |
| SA | GPRMMTEVVE CAAAELRVGM ELEAAFRPAG EVTVPVFRPR G (143) |
| MAP2 | GYQMLTNVVG VPPGDLRVGL RVR.VQFHTV AADVTLPYFT DETDGS (135) |
| | IDENTITY/SIMILARITY TO B3683 |
| MAP1 | 59/76% |
| RE | 36/52% |
| PSP | 33/53% |
| SA | 33/50% |
| MAP2 | 32/49% |
| (SEQ ID NO: 24) | |
| Gene sequence of cxgC | |
| (SEQ ID NO: 24) | |
| ATGGCGCTGGCACTCACCGATGAACAGGTACAGCTGACCGAGGCGATGGCGGGTTTCGCCCGCA | |
| GGCACGGCGGACTGGAACTGACCCGGTCGCAGTTCGACGCCCTCGCAGCCGGGGAACGCCCGGC | |
| GTTCTGGGCGGCCTTGGTCGCCAACGGACTGCACGGGGTTCAATTGCCCGAGCAGGGTGGGGGT | |
| TTCGTCGATGCCGCCTGCGTCATCGACGCCGCGGGCTACGGTCTGCTGCCCGGCCCGCTGCTGC | |
| CCACGATGATCGCCGGTGCCGTCATTGCAGACCTGCCGGAACAACCGGCGGTGCGCGCCGCGCG | |
| CGAGGCCCTCGCCGCGGGTGGCCCGATGGCGGTGTTGCTGCCGAGCGATGGCGTGCTGCGGGCC | |
| GAACCCGACGGCGCAGGGTGGCGGCTGACCGGCGCGGCCGGACCGCAGCTCGGCGTGGCCGCCG | |
| CGGAGCATGTGATCGTTGCCGCCGATACCGATGCGGCGCAAAGACTCTGGTTTCTGATCAACGC | |
| TGCCGGGCCGGGGGTGGTGGTGCAGGCGGCCGCCCCGACCGATCTGACCCGGGATGTCGGCACC | |
| CTGTCGTGCGCCGACGCACCCGTCGCGGCCGATGCCGTGCTGGCCGGTGTCGACCCGGTGCGGG | |
| CGCGGTGCCATGCGATCGGCCTGATGGCGGCCGAGGCAGCGGGGATCGCGCGCTGGTGTGTGGA | |
| CAATGTGGTCGCCTATCTGAAGGTGCGCGAACAGTTCGGACGCCGCATCGGGGCGTTCCAGGCC | |
| CTGCAGCACAAGGCGGCCATGCTGTTCATCGACAGTGAACTTGCCGCCGCCGCCGCATGGGATG | |
| CGGTGCGCGGCGCCGAACAACCGATCGAGCAACACGAGATCGCCGCCGCAGGCGCTGCCATCGC | |
| GGCGATCGGCAAGCTGCCGGATCTGGTGGTCGATGCGCTGACGATGTTCGGGGCCATCGGGTAC | |
| ACCTGGGAGCACGACCTGCACCTGTACTGGAAGCGGTCGATCAGCCTGGCCGCCGCCGCGGGCG | |
| GTGTCGCCGAATGGGCCGAGCTGCTCGGGGAACCCGACCGGCAGCCAAGAGATTTCGGCATCGA | |
| GCTGGCCGGTGTGGAAGAGCGGTTCCGGGGGCAGATCGCCGCGCTGATCGACGCCGCGGCGCAG | |
| CTGGACAACGAGGCGCCGGGCCGGCAGAACCCCGAGTACGAGGACTTCTGGACCGGTCCGCGCC | |
| GGACCGCACTGGCCGATGCCGGACTCGTCGCGCCATATCTGCCCGCGCCGTGGGGGCTGGACGC | |
| CACGCCGGCCCAACAGCTCGTCATCGACGAGGAATTCGACCGGCGGCCAACGCTTACCCGGCCA | |
| TCGTTGGGAATCGCACAGTGGATACTGCCGACGGTTATCGCCGAAGGCACCGACGGCCAACGGG | |
| AGCGCTTCGCGGTGCCGACGCTGCGCGGTGAGATCGGGTGGTGTCAGCTGTTCTCCGAACCCGG | |
| CGCCGGATCGGATCTGGCGTCCTTGACGACCAGGGCGACCAAGGTCGAGGGCGGCTGGCGGATC | |
| GACGGGCAGAAGGTGTGGACCTCCTCGGCGCAGCGCGCCGACTGGGGTGCGCTGCTGGCCAGGA | |
| CGGATCCGCAGGCCGCCAAGCACCGGGGCATCGGCTACTTCCTGATCGATATGACGAGCCCGGG | |
| CATCACCATCCGGCCGCTGCGAACCGCCAGCGGTGACGAGCATTTCAACGAGGTGTTCTTCGAC | |
| GATGTCTTCGTGCCCGATGACATGCTGGTCGGTGAGCCGACCGCGGGCTGGTCGCATGCGCTGG | |
| CCACGATGGCCAACGAACGGGTGGCCATCGGTGCCTACGCCAAACTGGACAAGGAACGTGAATT | |
| GCGGGCGCTGGCCCGTCAGGCCGGTCCGGCGGGTGTCATGGTGCGGCACGCGTTGGGCCGGGTA | |
| CGGGCCGCCACCAACGCCATCGGCGCGCTCGCGGTGCGCGACACCCTGCGCCGGCTCGCCGGAC | |
| ACGGGCCCGGCCCGGCGTCCAGCGTCGGCAAGGTCGGCACCGCACTGTTGGTGCGCCGGGTGAC | |
| CGCCGACGCGCTGGCTTTCAGCGGTCGGGCCGCCATGGTGGGTGGCGCCGACCACCCCGCAGTG | |
| GCCGACACGTTGATGATGCCTGCGGAGGTCATCGGCGGTGGCACCGTCGAGATCCAGCTCAATA | |
| TCATCGCCACCATGATCCTCGGACTACCGCGCGCA | |
| (SEQ ID NO: 25) | |
| protein sequence of CxgC | |
| (SEQ ID NO: 25) | |
| MALALTDEQVQLTEAMAGFARRHGGLELTRSQFDALAAGERPAFWAALVANGLHGVQLPEQGGG | |
| FVDAACVIDAAGYGLLPGPLLPTMIAGAVIADLPEQPAVRAAREALAAGGPMAVLLPSDGVLRA | |
| EPDGAGWRLTGAAGPQLGVAAAEHVIVAADTDAAQRLWFLINAAGPGVVVQAAAPTDLTRDVGT | |
| LSCADAPVAADAVLAGVDPVRARCHAIGLMAAEAAGIARWCVDNVVAYLKVREQFGRRIGAFQA | |
| LQHKAAMLFIDSELAAAAAWDAVRGAEQPIEQHEIAAAGAAIAAIGKLPDLVVDALTMFGAIGY | |
| TWEHDLHLYWKRSISLAAAAGGVAEWAELLGEPDRQPRDFGIELAGVEERFRGQIAALIDAAAQ | |
| LDNEAPGRQNPEYEDFWTGPRRTALADAGLVAPYLPAPWGLDATPAQQLVIDEEFDRRPTLTRP | |
| SLGIAQWILPTVIAEGTDGQRERFAVPTLRGEIGWCQLFSEPGAGSDLASLTTRATKVEGGWRI | |
| DGQKVWTSSAQRADWGALLARTDPQAAKHRGIGYFLIDMTSPGITIRPLRTASGDEHFNEVFFD | |
| DVFVPDDMLVGEPTAGWSHALATMANERVAIGAYAKLDKERELRALARQAGPAGVMVRHALGRV | |
| RAATNAIGALAVRDTLRRLAGHGPGPASSVGKVGTALLVRRVTADALAFSGRAAMVGGADHPAV | |
| ADTLMMPAEVIGGGTVEIQLNIIATMILGLPRA | |
| Alignment of Mycobacterium B3683 CxgC and homologs | |
| (SEQ ID NO: 25) | |
| B3683 = MYCOBACTERIUM B3683 CXGC | |
| (SEQ ID NO: 26) | |
| MAP = MYCOBACTERIUM AVIUM PARATUBERCULOSIS MAP4303C | |
| (SEQ ID NO: 27) | |
| NF = NOCARDIA_FARCINICA PUTATIVE ACYL COA DEHYDROGENASE | |
| (SEQ ID NO: 28) | |
| MT1 = MYCOBACTERIUM TUBERCULOSIS PROBABLE ACYL COA DEHYDROGENASE | |
| FADE34 | |
| (SEQ ID NO: 29) | |
| MT2 = MYCOBACTERIUM TUBERCULOSIS PROBABLE ACYL COA DEHYDROGENASE | |
| FADE6 | |
| (SEQ ID NO: 30) | |
| MT3 = MYCOBACTERIUM TUBERCULOSIS PROBABLE ACYL COA DEHYDROGENASE | |
| FADE22 | |
| | 1 50 |
| B3683 | ..MALALTDE QVQLTEAMAG FARRHGGLEL TRSQFDALAA GER....... |
| MAP | ..MTLGLSPE QQELGDAVGQ FAARNAPIAA TRDSFAELAA GRL....... |
| NF | MIVPVALTAD QAALAESVGG FAARHATREY TRRNTEQLKR GER....... |
| MT1 | ..MVATVTDE QSAARELVRG WARTAASGAA ATAAVRDMEY GFEEGNADAW |
| MT2 | ..MSIAITPE HYELADSVRS LVARVAPSEV LHAALESPVE NP........ |
| MT3 | ..MGIALTDD HRELSGVARA FLTSQKVRWA ARASLDAAG. DAR....... |
| | 51 100 |
| B3683 | PAFWAALVAN GLHGVQLPEQ GGG....FVD AACVIDAAGY GLLPGPLLPT |
| MAP | PRWWDGLVAN GFHAVHLPEE LGGQGGRLMD AACVLESAGK SLLPGPLLPT |
| NF | PAFWPELVAT GLTGVHLPDE VGGQGGAVAD IAVVVAEAGR ALLPGPLLPS |
| MT1 | RPVFAGLAGL GLFGVAVPED CGGAGGSIED LCAMVDEAAR ALVPGPVATT |
| MT2 | PPYWQAAAEQ GLQGVHLAES VGGQGFGILE LAVVLAEFGY GAVPGPFVPS |
| MT3 | PPFWQNLAEL GWLGLHIDER HGGSGYGLSE LVVVIEELGR AVAPGLFVPT |
| | 101 150 |
| B3683 | MIAGAVIADL PEQPAVRAAR EALAAGGPMA VLLPSDGVLR AEPDGAGWRL |
| MAP | VAAGAVALLA DPAPAARSVL RDLAAGIPAA VVLPGDGDLH AGAGDGHWLL |
| NF | VVASAIVATA ATGAGTEKAL RHFAEGGTGA VLLPEHGVAV SG...GEARL |
| MT1 | AVATLVVSDP KLR....... SALASGERFA GVAIDGGVQV DP...KTSTA |
| MT2 | AIASALIAAH DP...QAKVL AELATGAAIA AYALDSGLTA TRHG.DVLVI |
| MT3 | VIASAVVAKE GTDDQRARLL PALIDGTLTA GVGLDSQVQV TDG....VAD |
| | 151 200 |
| B3683 | TGAAGPQLGV AAAEHVIVAA DTDAAQRLWF LINAAGPGVV VQAAAPTDLT |
| MAP | SGASEVTAGV CAARIVLVGA RTRDGELVWA AVDTEKPTAT VEPISGTDLV |
| NF | SGRSGLVLGA PGAELFVVAA GSR.....WF LVERSAPGVG VEIEDGADLG |
| MT1 | SGTVGRVLGG APGGVVLLPA DGN.....WL LVDTACDEVV VEPLRATDFS |
| MT2 | RGEVRAVPAA AQASVLVLPV AIESR...DE WVVLRNDQLE IEAVKSLDPL |
| MT3 | .GEAGIVLGA GLAELLLVAA GDD.....VL VLERGRKGVS VDVPENFDPT |
| | 201 250 |
| B3683 | RDVGTLSCAD APVAADAVLA GVDPVRARCH AIGLMAAEAA GIARWCVDNV |
| MAP | ADAGVLRLDN HRVLDSEVLT GIDPERARCV VLGLVAATTA GVIQWCVQAV |
| NF | RDLG..RVAF QDVTPAAELD GIDGDRAADI AVAFLAVEAA GVIRWCSDTA |
| MT1 | LPLAR....M VLTSAPVTVL EVSGERVEDL AATVLAAEAA GVARWTLDTA |
| MT2 | RPIAHVRANA VDVSDDALLS NLTMTTAHAL MSTLLSAEAV GVARWATDTA |
| MT3 | RRSGRVRLDN VRVTTDDILL GAYES.ALAR ARTLLAAEAV GGAADCVDSA |
| | 251 300 |
| B3683 | VAYLKVREQF GRRIGAFQAL QHKAAMLFID SELAAAAAWD AVRGAEQPIE |
| MAP | TAHLRIREQF GKVIGTFQAL QHSAAMLLVS SELATAAAWD AVRAGDESLE |
| NF | TEYVQARKQF GRPIGAFQAV QHRTAQLLIT SELATAAAWD AVRGLDDEPD |
| MT1 | VAYAKVREQF GKPIGSFQAV KHLCAQMLCR AEQADVAAAD AARAAADSDG |
| MT2 | SAYAKIREQF GRPIGQFQAI KHKCAEMIAD TERATAAVWD AARALDDAGE |
| MT3 | VAYAKVRQQF GRTIATFQAV KHHCANMLVA AESAIAAVWD AARAAAEDEE |
| | 301 350 |
| B3683 | QH...EIAAA GAAIAAIGKL PDLVVDALTM FGAIGYTWEH DLHLYWKRSI |
| MAP | QH...RMAAA GAAVMAISPA PDLVLDALTM FGAIGFTWEH DLHLYWRRAI |
| NF | QR...AHAVA GAALITLGNA VHAAVECLAL HGAIGFTWEH DLHLYWRRAI |
| MT1 | TQLS..IAAA VAASIGIDAA KANAKDCIQV LGGIGCTWEH DAHLYLRRAH |
| MT2 | SSSDVEFAAA VAATLAPATA QRCTQDCIQV HGGIGFTWEH DTNVYYRRAL |
| MT3 | QF...RLAAA VAAALAFPAY ARNAELNIQV HGGIGFTWEH DAHLHLRRAL |
| | 351 400 |
| B3683 | SLAAAAGGVA EWAELLGEPD RQ..PRDFGI ELAGVEERFR GQIAALIDAA |
| MAP | SLAASIGPAN RWARRLGELT CTR.QRDMAV NLGDAESELR AKVAETLDAA |
| NF | TLAGLAGPGE RWERRLGEVA LRG.PRTFTV PLPETDTTFR QWVSGILDTA |
| MT1 | GIGGFLGGSG RWLRRVTALT QAGVRRRLGV DLAEVAG.LR PEIAAAVAEV |
| MT2 | MLAACFGRGS EYPQRVVDTA TTAGMRPVDI DLDPSTEKLR AQIRAEVAAL |
| MT3 | VTVGLFGGDA PVRDVFERTA AGV.TRAISL DLPAQAEELR ARIRSDAAEI |
| | 401 450 |
| B3683 | AQLDNEAPGR QNPEYEDFWT GPRRTALADA GLVAPYLPAP WGLDATPAQQ |
| MAP | LELRNDQPGR QG.DYSEFET GPQRTLISDA GLIAPHWPKP WGLDAGPLRQ |
| NF | AELTNPHPST IG.DHDSVNT GPRRTLLADH GLVSPPMPRP YGIEAGPLEQ |
| MT1 | AALPEE.... .......... .KRQVALADT GLLAPHWPAP YGRGASPAEQ |
| MT2 | KAMPRE.... .......... .PRTVAIAEG GWVLPYLPKP WGRAASPVEQ |
| MT3 | AALEKD.... .......... AQR.DKLIET GYVMPHWPRP WGRAAGAVEQ |
| | 451 500 |
| B3683 | LVIDEEFDRR PTLTRPSLGI AQWILPTVIA EGTDGQRERF AVPTLRGEIG |
| MAP | LIIDDEFAKR PALVRPSLGI AEWILPSVIR AAPKDLQEKL IPPTLRGEIA |
| NF | LILQDEYDR. HGIAQPSMGI GQWVVPIVLQ RGTPAQLERL AGPALRGEEI |
| MT1 | LLIDQELAA. AKVERPDLVI GWWAAPTILE HGTPEQIERF VPATMRGEFL |
| MT2 | IIIAQEFTA. GRVKRPQIAI ATWIVPSIVA FGTDNQKQRL LPPTFRGDIF |
| MT3 | LVIEEEFSA. AGIERPDYSI TGWVILTLIQ HGTPWQIERF VEKALRQQEI |
| | 501 550 |
| B3683 | WCQLFSEPGA GSDLASLTTR ATKVEGGWRI DGQKVWTSSA QRADWGALLA |
| MAP | WCQLFSEPGA GSDLAALSTR ATKVDGGWTI NGHKIWTSAA HRADYGALLA |
| NF | WCQLFSEPEA GSDVASLSLR ATKVDGGWQL NGQKIWTTLA HRSDWGLLLA |
| MT1 | WCQLFSEPGA GSDLASLRTK AVRADGGWLL TGQKVWTSAA HKARWGVCLA |
| MT2 | WCQLFSEPGA GSDLASLATK ATRVDGGWRI TGQKIWTTGA QYSQWGALLA |
| MT3 | WCQLFSEPDA GSDAASVKTR ATRVEGGWKI NGQKVWTSGA QYCARGLATV |
| | 551 600 |
| B3683 | RTDPQAAKHR GIGYFLIDMT SPGITIRPLR TASGDEHFNE VFFDDVFVPD |
| MAP | RTDPQAGKHR GIGYFVVDMR SAGIEVQPIK TATGDAHFNE VFLTDVFVPD |
| NF | RTDPEAERHR GLTMFLVDMH APGVDVRPIT QSSGDAEFNE VFFDDAFVPD |
| MT1 | RTDPDAPKHK GITYFLVDMT TPGIEIRPLR EITGDSLFNE VFLDNVFVPD |
| MT2 | RTDPSAPKHN GITYFLLDMK SEGVQVKPLR ELTGKEFFNT VYLDDVFVPD |
| MT3 | RTDPDAPKHA GITTVIIDML APGVEVRPLR QITGDSEFNE VFFNDVFVPD |
| | 601 650 |
| B3683 | DMLVGEPTAG WSHALATMAN ERVAIGAYAK LDKERELRAL ARQA.....G |
| MAP | DMLLGEPTGG WNLAIATMAE ERSAISGYVK FDRAAALRRL AAQP.....G |
| NF | DMVLGEPGQG WALTLETLAQ ERLFIGGVRD PGHNQRIREI IEREEY...A |
| MT1 | EMVVGAVNDG WRLARTTLAN ERVAMATGTA LGNPMEELLK VLGD.....M |
| MT2 | ELVLGEVNRG WEVSRNTLTA ERVSIGGSDS TFLPTLGEFV DFVRDYRFEG |
| MT3 | EDVVGAPNSG WTVARATLGN ERVSIGGSGS YYEAMAAKLV QLVQRR...S |
| | 651 700 |
| B3683 | PAGVMVRHAL GRVRAATNAI GALAVRDTLR RLAGHGPGPA SSVGKVGTAL |
| MAP | PDRDDALREL GRLDAYTTRS .........R RWECARPSGC STARRPGRRP |
| NF | GSRDEALRTL GRISARGAAI SAMNLRETIR RLDGQGVGPG TSIAKAAAAM |
| MT1 | ELDVAQQDRL GRLILLAQAG ALLDRRIAEL AVGGQDPGAQ SSVRKLIGVR |
| MT2 | QFDQVARHRA GQLIAEGHAT KLLNLRSTLL TLAGGDPMAP AAISKLLSMR |
| MT3 | DAFAGAPIRV GAFLAEDHAL RLLNLRRAAR SVEGAGPGPE GNITKLKVAE |
| | 701 750 |
| B3683 | LVRRVTADAL AFSGRAAMVG GAD..HPAVA DTLMMP.AEV IGGGTVEIQL |
| MAP | ASPRWR (678) |
| NF | LHTDAAAAAL ELIGPAAALS EAR..SEVVH HELDIP.TWV IGGGTLEIQL |
| MT1 | YRQALAEYLM EVSDGGGLVE NRA......V YDFLNTRCLT IAGGTEQILL |
| MT2 | TGQGYAEFAV SSFGTDAVIG DTERLPGKWG EYLLASRATT IYGGTSEVQL |
| MT3 | HMIEGAAIAA ALWGPEIALL DGP..GRVIG RTVMGARGMA IAGGTSEVTR |
| | 751 |
| B3683 | NIIATMILGL PRA (737) |
| MAP | |
| NF | NTIATLVMGL PRK (734) |
| MT1 | TVAAERLLGL PR (731) |
| MT2 | NIIAERLLGL PRDP (711) |
| MT3 | NQIAERILGM PRDPLIS (721) |
| | IDENTITY/SIMILARITY TO B3683 |
| MAP | 55/68% |
| NF | 47/61% |
| MT1 | 37/53% |
| MT2 | 39/51% |
| MT3 | 36/49% |
| (SEQ ID NO: 31) | |
| Gene sequence of cxgD | |
| (SEQ ID NO: 31) | |
| ATGACCACCGGCGACACCGAGCTGCCCGACTACAAGCGGGCCCGCCGGGCCCAGATCGTCGATG | |
| CGGCACTGGATCTGCTGAAGTCACAGGACTACGAGCAGATCCAGATGCGCGATGTCGCCGATCA | |
| CGCCCGAGTCGCATTGGGCACCCTGTACCGATACTTCAGCTCCAAGGAGCACGTTTACGCCGCG | |
| GTCCTGATGCAGTGGGCGCAACCGGTTTTCGCCGCGGCGGAAGCGGTCCGACCGGCCACCGAAC | |
| AGCAGGTCCGCGAGAAGATGCGCGGCATCATCACCAGCTTCGAACGTCGGCCGGCGTTCTTCAA | |
| GGTCTGCATGCTGTTGCAGAACACCACTGACGCCAATGCCCGCGACCTGATGGATCGATTCGCC | |
| TCCGTCGCCCAGCGCACCCTGGCCACGGACTTCGCCGCCATGGGCGAACAGGGATCGGCCGACA | |
| CCGCGATCATGGCCTGGGGCATCATCTCGACCATGCTGTCCGCGTCCATCCTGCGCGACCTGCC | |
| GATGGCCGACAC | |
| (SEQ ID NO: 32) | |
| protein sequence of CxgD | |
| (SEQ ID NO: 32) | |
| MTTGDTELPDYKRARRAQIVDAALDLLKSQDYEQIQMRDVADHARVALGTLYRYFSSKEHVYAA | |
| VLMQWAQPVFAAAEAVRPATEQQVREKMRGIITSFERRPAFFKVCMLLQNTTDANARDLMDRFA | |
| SVAQRTLATDFAAMGEQGSADTAIMAWGIISTMLSASILRDLPMAD | |
| Alignment of Mycobacterium B3683 CxgD and homologs | |
| (SEQ ID NO: 32) | |
| B3683 = Mycobacterium B3683 CxgD | |
| (SEQ ID NO: 33) | |
| NF = Nocardia farcinica putative transcriptional regulator | |
| (SEQ ID NO: 34) | |
| MT = Mycobacterium tuberculosis putative regulatory protein | |
| (SEQ ID NO: 35) | |
| RE = Rhodococcus erythropolis KstR | |
| (SEQ ID NO: 36) | |
| SA = Streptomyces avermitilis putative transcriptional regulator | |
| | 1 50 |
| B3683 | .......... .........M TTGDTELPDY KRARRAQIVD AALDLLKSQD |
| NF | .MASPSRSQP AAARPATVTT LSEDELSSAA QRERRKRILD ATLALASKGG |
| MT | .......... .......MAV LAESELGSEA QRERRKRILD ATMAIASKGG |
| RE | ........MM GATLPRIAEV RDAAEPSSDE QRARHVRMLE AAAELGTEKE |
| SA | MPAEAKVEAS TGARAARPAV QPASPPLTER QEARRRRILH ASAQLASRGG |
| | 51 100 |
| B3683 | YEQIQMRDVA DHARVALGTL YRYFSSKEHV YAAVLMQWAQ PVFAA...AE |
| NF | YDAVQMRAVA ERADVAVGTL YRYFPSKVHL LVSALAREFE QFESK..RKP |
| MT | YEAVQMRAVA DRADVAVGTL YRYFPSKVHL LVSALGREFS RIDAKTDRSA |
| RE | LSRVQMHEVA KRAGVAIGTL YRYFPSKTHL FVAVMVEQID QIGDSFAKHQ |
| SA | FDAVQMREVA ESSQVALGTL YRYFPSKVHL LVATMQAQLE HMHGTLRKKP |
| | 101 150 |
| B3683 | AVRPATEQQV REKMRGIITS FERRPAFFKV CMLLQNTTDA NARDLMDRFA |
| NF | LAGATPRERM HLLLTQITRM MQRDPLLTEA MTRAFMFADA SAAAEVDRVG |
| MT | VAGATPFQRL NFMVGKLNRA MQRNPLLTEA MTRAYVFADA SAASEVDQVE |
| RE | VQSANPQDAV YEVLVRATRG LLRRPALSTA MLQSSSTANV ATVPDVGKID |
| SA | PAGDTAAERV AETLMRAFRA LQREPHLADA MVRALTFADR SVSPEVDQVS |
| | 151 200 |
| B3683 | SVAQRTLATD FAAMG.EQGS ADTAIMAWGI ISTMLSASIL RDLPMAD (174) |
| NF | KVMDRVFARA MNDGEPDERQ LAIARVISDV WLSNLVAWLT RRASATDVSD |
| MT | KLIDSMFARA MANGEPTEDQ YHIARVISDV WLSNLLAWLT RRASATDVSK |
| RE | RGFRQIILDA AGIENPTEED NTGLRLLMQL WFGVIQSCLN GRISIPDAEY |
| SA | RQTTVIILDA MGLDDPTPEQ LSAVRVIEHT WHSALITWLS GRASIAQVKI |
| | 201 |
| B3683 | |
| NF | RLELTVDLLL GDKE (208) |
| MT | RLDLAVRLLI GDQDSA (211) |
| RE | DIRKGCDLLL VNLSRH (199) |
| SA | DIETVCRLID LTEADETP (218) |
| | IDENTITY/SIMILARITY TO B3683 |
| NF | 34/50% |
| MT | 33/48% |
| RE | 32/53% |
| SA | 28/48% |

A number of embodiments of the invention have been described. Nevertheless, it will be understood that various modifications may be made without departing from the spirit and scope of the invention. Accordingly, other embodiments are within the scope of the following claims.

What is claimed is:

1. An isolated, synthetic or recombinant nucleic acid comprising:
(a) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:1, and having a KsdA polypeptide or a 3-ketosteroid-Δ1-dehydrogenase activity;
(b) a nucleic acid sequence encoding a polypeptide having an amino acid sequence as set forth in SEQ ID NO:2, and having a KsdA polypeptide or 3-ketosteroid-Δ1-dehydrogenase activity, and enzymatically active fragments thereof;
(c) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:9, and having a CxgA polypeptide or an acetyl CoA-acetyltransferase/thiolase activity;
(d) a nucleic acid sequence encoding a polypeptide having an amino acid sequence as set forth in SEQ ID NO:10 or SEQ ID NO:11, and having a CxgA polypeptide or an acetyl CoA-acetyltransferase/thiolase activity, and enzymatically active fragments thereof;
(e) a nucleic acid sequence encoding a polypeptide having at least about 75%, 76%, 77s %, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:17, and having a CxgB polypeptide or a DNA-binding protein activity;
(f) a nucleic acid sequence encoding a polypeptide having an amino acid sequence as set forth in SEQ ID NO:18, and having a CxgB polypeptide or a DNA-binding protein activity, and DNA-binding active fragments thereof;
(g) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:24, and having a CxgC polypeptide or a DNA-binding protein activity;
(h) a nucleic acid sequence encoding a polypeptide having an amino acid sequence as set forth in SEQ ID NO:25, and having a CxgC polypeptide or an acyl-CoA dehydrogenase/FadE activity, and enzymatically active fragments thereof;
(i) a nucleic acid sequence encoding a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:31, and having a CxgD polypeptide or a TetR-like regulatory protein/KstR activity;
(j) a nucleic acid sequence encoding a polypeptide having an amino acid sequence as set forth in SEQ ID NO:32, and having a CxgD polypeptide or a TetR-like regulatory protein/KstR activity, and enzymatically active fragments thereof;
(k) the nucleic acid of any of (a) to (j), wherein the sequence identities are determined by analysis with a sequence comparison algorithm or by a visual inspection;
(l) the nucleic acid of (k), wherein the sequence comparison algorithm is a BLAST version 2.2.2 algorithm where a filtering setting is set to blastall-p blastp-d “nr pataa”-F F, and all other options are set to default, or a FASTA version 3.0t78, with the default parameters;
(m) a nucleic acid sequence that hybridizes under stringent conditions to a nucleic acid consisting of SEQ ID NO:1, SEQ ID NO:9, SEQ ID NO:17, SEQ ID NO:24 and/or SEQ ID NO:31, and the nucleic acid encodes a polypeptide having a KsdA polypeptide or 3-ketosteroid-Δ1-dehydrogenase activity, a CxgA polypeptide or an acetyl CoA-acetyltransferase/thiolase activity, a CxgB polypeptide or a DNA-binding protein activity, a CxgC polypeptide or an acyl-CoA dehydrogenase/FadE activity, or a CxgD polypeptide or a TetR-like regulatory protein/KstR activity, respectively,
wherein the stringent conditions include a wash step comprising a wash in 0.2×SSC at a temperature of about 65° C. for about 15 minutes;
(n) the nucleic acid of any of (a) to (m) encoding a polypeptide lacking a signal sequence or proprotein sequence, or lacking a homologous promoter sequence;
(o) the nucleic acid of any of (a) to (n) further comprising a sequence encoding a heterologous amino acid sequence, or the nucleic acid further comprises a heterologous nucleotide sequence;
(p) the nucleic acid of (o) wherein the heterologous amino acid sequence comprises, or consists of a sequence encoding a heterologous (leader) signal sequence, or a tag or an epitope, or the heterologous nucleotide sequence comprises a heterologous promoter sequence;
(q) the nucleic acid of (p) or (p), wherein the heterologous nucleotide sequence encodes a heterologous (leader) signal sequence comprising or consisting of an N-terminal and/or C-terminal extension for targeting to an endoplasmic reticulum (ER) or endomembrane, or to a bacterial endoplasmic reticulum (ER) or endomembrane system, or the heterologous sequence encodes a restriction site;
(r) the nucleic acid of (p), wherein the heterologous promoter sequence comprises or consists of a constitutive or inducible promoter, or a cell type specific promoter, or a plant specific promoter, or a bacteria specific promoter, or a Mycobacterium specific promoter;
(s) the nucleic acid of any of (a) to (r), wherein the enzyme activity is thermotolerant; or
(t) a nucleic acid sequence completely complementary to the nucleotide sequence of any of (a) to (s).

2. A probe for isolating or identifying a KsdA, CxgA, CxgB, CxgC or CxgD-encoding nucleic acid comprising a nucleic acid of claim 1.

3. A vector, expression cassette or cloning vehicle: (a) comprising the nucleic acid (polynucleotide) sequence of claim 1; or, (b) the vector, expression cassette or cloning vehicle of (a) comprising or contained in a viral vector, a plasmid, a phage, a phagemid, a cosmid, a fosmid, a bacteriophage, an artificial chromosome, an adenovirus vector, a retroviral vector or an adeno-associated viral vector; or, a bacterial artificial chromosome (BAC), a plasmid, a bacteriophage P1-derived vector (PAC), a yeast artificial chromosome (YAC), or a mammalian artificial chromosome (MAC).

4. A host cell or a transformed cell: (a) comprising the nucleic acid (polynucleotide) sequence of claim 1, or the vector, expression cassette or cloning vehicle of claim 3; or, (b) the host cell or a transformed cell of (a), wherein the cell is a bacterial cell, a mammalian cell, a fungal cell, a yeast cell, an insect cell or a plant cell.

5. A transgenic non-human animal: (a) comprising the nucleic acid (polynucleotide) sequence of claim 1; the vector, expression cassette or cloning vehicle of claim 3; or the host cell or a transformed cell of claim 4; or (b) the transgenic non-human animal of (a), wherein the animal is a mouse, a rat, a goat, a rabbit, a sheep, a pig or a cow.

6. A transgenic plant or seed: (a) comprising the nucleic acid (polynucleotide) sequence of claim 1; the vector, expression cassette or cloning vehicle of claim 3; or the host cell or a transformed cell of claim 4; (b) the transgenic plant of (a), wherein the plant is a corn plant, a sorghum plant, a potato plant, a tomato plant, a wheat plant, an oilseed plant, a rapeseed plant, a soybean plant, a rice plant, a barley plant, a grass, a cottonseed, a palm, a sesame plant, a peanut plant, a sunflower plant or a tobacco plant; the transgenic seed of (a), wherein the seed is a corn seed, a wheat kernel, an oilseed, a rapeseed, a soybean seed, a palm kernel, a sunflower seed, a sesame seed, a rice, a barley, a peanut, a cottonseed, a palm, a peanut, a sesame seed, a sunflower seed or a tobacco plant seed.

7. An antisense oligonucleotide comprising a nucleic acid sequence complementary to or capable of hybridizing under stringent conditions to the nucleic acid (polynucleotide) sequence of claim 1.

8. A method of inhibiting the translation of a message (mRNA) in a cell comprising administering to the cell or expressing in the cell an antisense oligonucleotide comprising the nucleic acid (polynucleotide) sequence of claim 1.

9. An isolated, synthetic or recombinant polypeptide comprising:
(a) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:2, and enzymatically active fragments thereof, and having a ksdA polypeptide or a 3-ketosteroid-Δ1-dehydrogenase activity;
(b) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:10 or SEQ ID NO:11, and enzymatically active fragments thereof, and having a cxgA polypeptide or an acetyl CoA-acetyltransferase/thiolase activity;
(c) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:18, and enzymatically active fragments thereof, and having a cxgB polypeptide or a DNA-binding protein activity;
(d) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:25, and enzymatically active fragments thereof, and having a cxgC polypeptide or a DNA-binding protein activity;
(e) a polypeptide having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:32, and enzymatically active fragments thereof, and having a cxgD polypeptide or a TetR-like regulatory protein/KstR activity;
(f) the polypeptide of any of (a) to (e), wherein the sequence identities are determined by analysis with a sequence comparison algorithm or by a visual inspection;
(g) the polypeptide of (f), wherein the sequence comparison algorithm is a BLAST version 2.2.2 algorithm where a filtering setting is set to blastall-p blastp-d “nr pataa”-F F, and all other options are set to default, or a FASTA version 3.0t78, with the default parameters;
(h) a polypeptide encoded by the nucleic acid of any of claim 1(a) to claim 1(s);
(i) the polypeptide of any of (a) to (h), lacking a signal sequence or proprotein sequence;
(j) the polypeptide of any of (a) to (i) further comprising a heterologous amino acid sequence;
(k) the polypeptide of (j) wherein the heterologous amino acid sequence comprises, or consists of, a heterologous (leader) signal sequence, or a tag or an epitope;
(l) the polypeptide of (j), wherein the heterologous (leader) signal sequence comprises or consists of an N-terminal and/or C-terminal extension for targeting to an endoplasmic reticulum (ER) or endomembrane, or to a bacterial endoplasmic reticulum (ER) or endomembrane system;
(m) the polypeptide of any of (a) to (l), wherein the enzyme activity is thermotolerant; or
(n) the polypeptide of any of (a) to (m), wherein the polypeptide is glycosylated, or the polypeptide comprises at least one glycosylation site, (ii) the polypeptide of (i) wherein the glycosylation is an N-linked glycosylation or an O-linked glycosylation; (iii) the polypeptide of (i) or (ii) wherein the polypeptide is glycosylated after being expressed in a yeast cell.

10. A protein preparation comprising the polypeptide of claim 9, wherein the protein preparation comprises a liquid, a solid or a gel.

11. A heterodimer: (a) comprising the polypeptide of claim 9 and a second domain; or (b) the heterodimer of (a), wherein the second domain is a polypeptide and the heterodimer is a fusion protein, or the second domain is an epitope or a tag.

12. A homodimer comprising the polypeptide of claim 9.

13. An immobilized polypeptide: (a) wherein the polypeptide comprises the polypeptide of claim 9; or, (b) the immobilized polypeptide of (a), wherein the polypeptide is immobilized on a cell, a metal, a resin, a polymer, a ceramic, a glass, a microelectrode, a graphitic particle, a bead, a gel, a plate, an array or a capillary tube.

14. An isolated, synthetic or recombinant antibody: (a) that specifically binds to the polypeptide of claim 9; or, (b) the isolated, synthetic or recombinant antibody of (a), wherein the antibody is a monoclonal or a polyclonal antibody, or antigen binding fragment thereof.

15. A hybridoma comprising the antibody of claim 14.

16. An array comprising an immobilized nucleic acid, polypeptide and/or antibody, wherein the nucleic acid comprises the nucleic acid of claim 1, or the polypeptide comprises the polypeptides as set forth in 1; and/or the antibody comprises the antibody of claim 14, or a combination thereof.

17. A method of isolating or identifying a polypeptide having a KsdA, CxgA, CxgB, CxgC or CxgD activity, comprising:
(a) providing the antibody of claim 14;
(b) providing a sample comprising polypeptides; and
(c) contacting the sample of step (b) with the antibody of step (a) under conditions wherein the antibody can specifically bind to the polypeptide, thereby isolating or identifying a polypeptide having a KsdA, CxgA, CxgB, CxgC or CxgD activity.

18. A method of making an anti-KsdA, CxgA, CxgB, CxgC or CxgD antibody comprising administering to a non-human animal:
(a) the KsdA, CxgA, CxgB, CxgC or CxgD-encoding nucleic acid (polynucleotide) sequence of claim 1 in an amount sufficient to generate a humoral immune response, thereby making an anti-KsdA, CxgA, CxgB, CxgC or CxgD antibody; or
(b) the polypeptide of claim 9 in an amount sufficient to generate a humoral immune response, thereby making an anti-KsdA, CxgA, CxgB, CxgC or CxgD antibody.

19. A method of producing a recombinant polypeptide comprising:
(A) (a) providing a nucleic acid operably linked to a promoter, wherein the nucleic acid comprises the nucleic acid (polynucleotide) sequence of claim 1; and (b) expressing the nucleic acid of step (a) under conditions that allow expression of the polypeptide, thereby producing a recombinant polypeptide; or
(B) the method of (A), further comprising transforming a host cell with the nucleic acid of step (a) followed by expressing the nucleic acid of step (a), thereby producing a recombinant polypeptide in a transformed cell.

20. A method for identifying a polypeptide having KsdA, CxgA, CxgB, CxgC or CxgD activity comprising:
(a) providing the polypeptide of claim 9;
(b) providing a KsdA, CxgA, CxgB, CxgC or CxgD binding protein or substrate; and
(c) contacting the polypeptide with the substrate of step (b) and detecting a decrease in the amount of substrate or an increase in the amount of a reaction product, wherein a decrease in the amount of the substrate or an increase in the amount of the reaction product detects a polypeptide having a KsdA, CxgA, CxgB, CxgC or CxgD activity.

21. A method for identifying a KsdA, CxgA, CxgB, CxgC or CxgD binding protein or substrate comprising:
(a) providing a KsdA, CxgA, CxgB, CxgC or CxgD polypeptide of claim 9;
(b) providing a test binding protein or substrate; and
(c) contacting the KsdA, CxgA, CxgB, CxgC or CxgD polypeptide of step (a) with the test binding protein or substrate of step (b) and detecting a decrease in the amount of binding protein or substrate or an increase in the amount of reaction product, wherein a decrease in the amount of the substrate or an increase in the amount of a reaction product identifies the test substrate as a KsdA, CxgA, CxgB, CxgC or CxgD binding protein or substrate.

22. A method of determining whether a test compound specifically binds to a KsdA, CxgA, CxgB, CxgC or CxgD polypeptide comprising:
(a) expressing a nucleic acid or a vector comprising the nucleic acid under conditions permissive for translation of the nucleic acid to a polypeptide, wherein the nucleic acid has the nucleic acid (polynucleotide) sequence of claim 1;
(b) providing a test compound;
(c) contacting the KsdA, CxgA, CxgB, CxgC or CxgD polypeptide with the test compound; and
(d) determining whether the test compound of step (b) specifically binds to the KsdA, CxgA, CxgB, CxgC or CxgD polypeptide.

23. A method of determining whether a test compound specifically binds to a KsdA, CxgA, CxgB, CxgC or CxgD polypeptide comprising:
(a) providing the KsdA, CxgA, CxgB, CxgC or CxgD polypeptide of claim 9;
(b) providing a test compound;
(c) contacting the polypeptide with the test compound; and
(d) determining whether the test compound of step (b) specifically binds to the ksdA, cxgA, cxgB, cxgC or cxgD polypeptide.

24. A method for identifying a modulator of a KsdA, CxgA, CxgB, CxgC or CxgD polypeptide comprising:
(A) (a) providing the KsdA, CxgA, CxgB, CxgC or CxgD polypeptide of claim 9;
(b) providing a test compound;
(c) contacting the polypeptide of step (a) with the test compound of step (b) and measuring an activity of the KsdA, CxgA, CxgB, CxgC or CxgD polypeptide, wherein a change in the KsdA, CxgA, CxgB, CxgC or CxgD activity measured in the presence of the test compound compared to the activity in the absence of the test compound provides a determination that the test compound modulates the KsdA, CxgA, CxgB, CxgC or CxgD activity;
(B) the method of (A), wherein the KsdA, CxgA, CxgB, CxgC or CxgD activity is measured by providing a KsdA, CxgA, CxgB, CxgC or CxgD substrate and detecting a decrease in the amount of the substrate or an increase in the amount of a reaction product, or, an increase in the amount of the substrate or a decrease in the amount of a reaction product;
(c) the method of (B), wherein a decrease in the amount of the substrate or an increase in the amount of the reaction product with the test compound as compared to the amount of substrate or reaction product without the test compound identifies the test compound as an activator of KsdA, CxgA, CxgB, CxgC or CxgD activity; or,
(d) the method of (B), wherein an increase in the amount of the substrate or a decrease in the amount of the reaction product with the test compound as compared to the amount of substrate or reaction product without the test compound identifies the test compound as an inhibitor of KsdA, CxgA, CxgB, CxgC or CxgD activity.

25. A computer system comprising:
(a) a processor and a data storage or a machine readable memory device wherein said data storage device has stored thereon a polypeptide sequence or a nucleic acid sequence, wherein the polypeptide sequence comprises the polypeptide (amino acid) sequence of claim 9, a polypeptide encoded by the nucleic acid (polynucleotide) sequence of claim 1;
(b) the computer system of (a), further comprising a sequence comparison algorithm and a data storage device or machine readable memory device having at least one reference sequence stored thereon;
(c) the computer system of (b), wherein the sequence comparison algorithm comprises a computer program that indicates polymorphisms; or
(d) the computer system of any of (a) to (c), further comprising an identifier that identifies one or more features in said sequence.

26. A computer readable medium or a machine readable memory device having stored thereon a polypeptide sequence or a nucleic acid sequence, wherein the polypeptide sequence comprises the polypeptide (amino acid) sequence of claim 9; a polypeptide encoded by the nucleic acid (polynucleotide) sequence of claim 1.

27. A method for identifying a feature in a sequence comprising: (a) reading the sequence using a computer program functionally saved (embedded in) a computer or a machine readable memory device, wherein the computer program identifies one or more features in a sequence, wherein the sequence comprises a polypeptide sequence or a nucleic acid sequence, wherein the polypeptide sequence comprises the polypeptide (amino acid) sequence of claim 9; a polypeptide encoded by the nucleic acid (polynucleotide) sequence of claim 1; and, (b) identifying one or more features in the sequence with the computer program.

28. A method for isolating or recovering a nucleic acid encoding a polypeptide with a KsdA, CxgA, CxgB, CxgC or CxgD activity from a sample comprising:
(A) (a) providing a polynucleotide probe comprising the nucleic acid (polynucleotide) sequence of claim 1;
(b) isolating a nucleic acid from the sample or treating the sample such that nucleic acid in the sample is accessible for hybridization to a polynucleotide probe of step (a);
(c) combining the isolated nucleic acid or the treated sample of step (b) with the polynucleotide probe of step (a); and
(d) isolating a nucleic acid that specifically hybridizes with the polynucleotide probe of step (a), thereby isolating or recovering a nucleic acid encoding a polypeptide with a KsdA, CxgA, CxgB, CxgC or CxgD activity from a sample;
(B) the method of (A), wherein the sample is or comprises an environmental sample;
(C) the method of (B), wherein the environmental sample is or comprises a water sample, a liquid sample, a soil sample, an air sample or a biological sample; or
(D) the method of (C), wherein the biological sample is derived from a bacterial cell, a protozoan cell, an insect cell, a yeast cell, a plant cell, a fungal cell or a mammalian cell.

29. A method of generating a variant of a nucleic acid encoding a polypeptide with a KsdA, CxgA, CxgB, CxgC or CxgD activity comprising:
(A) (a) providing a template nucleic acid comprising the nucleic acid (polynucleotide) sequence of claim 1; and
(b) modifying, deleting or adding one or more nucleotides in the template sequence, or a combination thereof, to generate a variant of the template nucleic acid.
(B) the method of (A), further comprising expressing the variant nucleic acid to generate a variant KsdA, CxgA, CxgB, CxgC or CxgD polypeptide;
(C) the method of (A) or (B), wherein the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR) and a combination thereof;
(D) the method of any of (A) to (C), wherein the modifications, additions or deletions are introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof;
(E) the method of any of (A) to (D), wherein the method is iteratively repeated until a (variant) KsdA, CxgA, CxgB, CxgC or CxgD polypeptide having an altered or different (variant) activity, or an altered or different (variant) stability from that of a polypeptide encoded by the template nucleic acid is produced, or an altered or different (variant) secondary structure from that of a polypeptide encoded by the template nucleic acid is produced, or an altered or different (variant) post-translational modification from that of a polypeptide encoded by the template nucleic acid is produced;
(F) the method of (E), wherein the variant KsdA, CxgA, CxgB, CxgC or CxgD polypeptide is thermotolerant, and retains some activity after being exposed to an elevated temperature;
(G) the method of (E), wherein the variant KsdA, CxgA, CxgB, CxgC or CxgD polypeptide has increased glycosylation as compared to the KsdA, CxgA, CxgB, CxgC or CxgD activity encoded by a template nucleic acid;
(H) the method of (E), wherein the variant KsdA, CxgA, CxgB, CxgC or CxgD polypeptide has a KsdA, CxgA, CxgB, CxgC or CxgD activity under a high temperature, wherein the KsdA, CxgA, CxgB, CxgC or CxgD polypeptide encoded by the template nucleic acid is not active under the high temperature;
(I) the method of any of (A) to (H), wherein the method is iteratively repeated until a KsdA, CxgA, CxgB, CxgC or CxgD polypeptide coding sequence having an altered codon usage from that of the template nucleic acid is produced; or
(J) the method of any of (A) to (H), wherein the method is iteratively repeated until a ksdA, cxgA, cxgB, cxgC or cxgD gene having higher or lower level of message expression or stability from that of the template nucleic acid is produced.

30. A method for modifying codons in a nucleic acid encoding a polypeptide with a KsdA, CxgA, CxgB, CxgC or CxgD activity to increase its expression in a host cell, the method comprising:
(a) providing a nucleic acid encoding a polypeptide with a KsdA, CxgA, CxgB, CxgC or CxgD activity comprising the nucleic acid (polynucleotide) sequence of claim 1; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

31. A method for modifying codons in a nucleic acid encoding a KsdA, CxgA, CxgB, CxgC or CxgD polypeptide, the method comprising:
(a) providing a nucleic acid encoding a polypeptide with a KsdA, CxgA, CxgB, CxgC or CxgD activity comprising the nucleic acid (polynucleotide) sequence of claim 1; and,
(b) identifying a codon in the nucleic acid of step (a) and replacing it with a different codon encoding the same amino acid as the replaced codon, thereby modifying codons in a nucleic acid encoding a KsdA, CxgA, CxgB, CxgC or CxgD polypeptide.

32. A method for modifying codons in a nucleic acid encoding a KsdA, CxgA, CxgB, CxgC or CxgD polypeptide to increase its expression in a host cell, the method comprising:
(a) providing a nucleic acid encoding a KsdA, CxgA, CxgB, CxgC or CxgD polypeptide comprising the nucleic acid (polynucleotide) sequence of claim 1; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

33. A method for modifying a codon in a nucleic acid encoding a polypeptide having a KsdA, CxgA, CxgB, CxgC or CxgD activity to decrease its expression in a host cell, the method comprising:
(A) (a) providing a nucleic acid encoding a KsdA, CxgA, CxgB, CxgC or CxgD polypeptide comprising the nucleic acid (polynucleotide) sequence of claim 1; and
(b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell; or
(B) the method of (A), wherein the host cell is a bacterial cell, a fungal cell, an insect cell, a yeast cell, a plant cell or a mammalian cell.

34. A method of increasing thermotolerance or thermostability of a KsdA, CxgA, CxgB, CxgC or CxgD polypeptide, the method comprising glycosylating a KsdA, CxgA, CxgB, CxgC or CxgD polypeptide, wherein the polypeptide comprises at least thirty contiguous amino acids of the polypeptide of claim 9, or a polypeptide encoded by the nucleic acid (polynucleotide) sequence of claim 1, thereby increasing the thermotolerance or thermostability of the KsdA, CxgA, CxgB, CxgC or CxgD polypeptide.

35. A method for overexpressing a recombinant KsdA, CxgA, CxgB, CxgC or CxgD polypeptide in a cell comprising expressing a vector comprising the nucleic acid (polynucleotide) sequence of claim 1, wherein overexpression is effected by use of a high activity promoter, a dicistronic vector or by gene amplification of the vector.

36. A method of making a transgenic plant comprising:
(A) (a) introducing a heterologous nucleic acid sequence into the cell, wherein the heterologous nucleic sequence comprises the nucleic acid (polynucleotide) sequence of claim 1, thereby producing a transformed plant cell; and (b) producing a transgenic plant from the transformed cell;
(B) the method of (A), wherein the step (A)(a) further comprises introducing the heterologous nucleic acid sequence by electroporation or microinjection of plant cell protoplasts; or
(C) the method of (C), wherein the step (A)(a) comprises introducing the heterologous nucleic acid sequence directly to plant tissue by DNA particle bombardment or by using an Agrobacterium tumefaciens host.

37. A method of expressing a heterologous nucleic acid sequence in a plant cell comprising the following steps:
(a) transforming the plant cell with a heterologous nucleic acid sequence operably linked to a promoter, wherein the heterologous nucleic sequence comprises the nucleic acid (polynucleotide) sequence of claim 1; and
(b) growing the plant under conditions wherein the heterologous nucleic acids sequence is expressed in the plant cell.

38. A process for modulating the production of androstenedione (AD, or 4-androstenedione), androstadienedione (ADD, or 1,4-androstadiene-3,17-dione), 20-(hydroxymethyl)pregna-4-en-3-one and/or 20-(hydroxymethyl)pregna-1,4-dien-3-one in a cell, comprising:
(a) (i) over- or underexpressing any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell, or (ii) deleting expression of any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell;
(b) the process of (a) wherein the cell is a prokaryotic cell or a eukaryotic cell;
(c) the process of (b) wherein the prokaryotic cell is a bacterial cell, or the eukaryotic cell is a yeast or fungal cell;
(d) the process of (c), wherein the bacterial cell is a member of the genus Actinobacteria, or a member of the family Mycobacteriaceae;
(e) the process of (d), wherein the member of the family Mycobacteriaceae is a Mycobacterium strain designated B3683 and/or B3805, or Mycobacterium ATCC 29472;
(f) the process of any of (a) to (e), wherein the any one, or several of, or all of KsdA-, CxgA-, CxgB-, CxgC- and/or CxgD-encoding nucleic acids are over- or underexpressed by a process comprising deleting, mutating or disrupting a transcriptional control sequence for a ksdA, cxgA, cxgB, cxgC and/or cxgD gene,
wherein the deleting, mutating or disrupting of the transcriptional control sequence results in the overexpression and/or the underexpression of the ksdA, cxgA, cxgB, cxgC and/or cxgD gene, and/or overexpression and/or the underexpression of the KsdA-, CxgA-, CxgB-, CxgC- and/or CxgD polypeptide-encoding message (mRNA);
(g) the process of (f), wherein the transcriptional control sequence is a promoter and/or an enhancer;
(h) the process of any of (a) to (e), wherein the any one, or several of, or all of KsdA-, CxgA-, CxgB-, CxgC- and/or CxgD-encoding nucleic acids are over- or underexpressed by a process comprising deleting, mutating or disrupting a trans-acting factor that regulates transcription of a ksdA, cxgA, cxgB, cxgC and/or cxgD gene,
wherein the deleting, mutating or disrupting of the trans-acting factor results in the overexpression and/or the underexpression of the ksdA, cxgA, cxgB, cxgC and/or cxgD gene;
(i) the process of any of (a) to (e), wherein the any one, or several of, or all of KsdA-, CxgA-, CxgB-, CxgC- and/or CxgD-encoding nucleic acids are over- or underexpressed by a process comprising upregulating, deleting, mutating or disrupting a message (mRNA) of a KsdA-, CxgA-, CxgB-, CxgC- and/or CxgD-encoding nucleic acid,
wherein the upregulating, deleting, mutating or disrupting of the message (mRNA) results in the overexpression and/or the underexpression of the KsdA-, CxgA-, CxgB-, CxgC- and/or CxgD polypeptides;
(j) the process of (i), wherein the expression of a message (mRNA) of a KsdA-, CxgA-, CxgB-, CxgC- and/or CxgD-encoding nucleic acid is deleted or disrupted by an antisense, ribozyme and/or RNAi specific for a message (mRNA) of a KsdA-, CxgA-, CxgB-, CxgC- and/or CxgD-encoding nucleic acid;
(k) the process of any of (a) to (e), wherein the any one, or several of, or all of the KsdA-, CxgA-, CxgB-, CxgC- and/or CxgD polypeptides in the cell are over- or underexpressed by addition of an inhibitor or activator of the activity of the KsdA-, CxgA-, CxgB-, CxgC- and/or CxgD polypeptide;
(l) the process of (k), wherein the inhibitor or activator of the activity of the KsdA-, CxgA-, CxgB-, CxgC- and/or CxgD polypeptide is a small molecule or an antibody inhibitor or activator of the activity of the KsdA-, CxgA-, CxgB-, CxgC- and/or CxgD polypeptide;
(m) the process of any of (a) to (l), wherein the KsdA-, CxgA-, CxgB-, CxgC- and/or CxgD-encoding nucleic acid comprises a nucleic acid as set forth in claim 1; or
(n) the process of any of (a) to (l), wherein the KsdA-, CxgA-, CxgB-, CxgC- and/or CxgD polypeptide comprises a polypeptide as set forth in claim 9.

39. A cell-based process for producing an androstenedione (AD, or 4-androstene-3,17-dione) of relative purity, or substantially free of androstadienedione (ADD, or 1,4-androstadiene-3,17-dione), 20-(hydroxymethyl)pregna-4-en-3-one and/or 20-(hydroxymethyl)pregna-1,4-dien-3-one, comprising
(a) (i) making a cell that underexpresses (as compared to a wild type cell) or does not express any one, or several of, or all of KsdA-, CxgA-, CxgB-, CxgC- and/or CxgD-encoding nucleic acids and/or KsdA-, CxgA-, CxgB-, CxgC- and/or CxgD polypeptides in the cell; and, (ii) culturing the cell under conditions wherein the androstenedione is produced,
wherein underexpressing the KsdA-, CxgA-, CxgB-, CxgC- and/or CxgD-encoding nucleic acids and/or KsdA-, CxgA-, CxgB-, CxgC- and/or CxgD polypeptides in the cell results production of an androstenedione (AD) of relative purity, or substantially free of androstadienedione (ADD), 20-(hydroxymethyl)pregna-4-en-3-one and/or 20-(hydroxymethyl)pregna-1,4-dien-3-one; or
(b) the process of (a), wherein the underexpression of the KsdA-, CxgA-, CxgB-, CxgC- and/or CxgD-encoding nucleic acids and/or the KsdA-, CxgA-, CxgB-, CxgC- and/or CxgD polypeptides in the cell is made by practicing the method of claim 38;
(c) the process of (a) or (b), wherein the cell underexpresses a KsdA-, CxgA-, CxgB-, CxgC- and/or CxgD-encoding nucleic acid (as compared to a wild type or unmanipulated cell) by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more;
(d) the process of (a) or (b), wherein the cell produces (generates) an androstenedione (AD) of relative greater purity, or substantially free of androstadienedione (ADD), 20-(hydroxymethyl)pregna-4-en-3-one and/or 20-(hydroxymethyl)pregna-1,4-dien-3-one by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;
(e) the process of any of (a) to (d), wherein the cell produces at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more % fewer (lesser amounts of) impurities in the AD synthesis process; or
(f) the process of (e), wherein the fewer impurities comprise fewer (lesser amounts of) androstadienedione (ADD), 20-(hydroxymethyl)pregna-4-en-3-one and/or 20-(hydroxymethyl)pregna-1,4-dien-3-one.

40. A cell-based process for producing an androstenedione (AD, or 4-androstene-3,17-dione) of relative purity, or substantially free of androstadienedione (ADD, or 1,4-androstadiene-3,17-dione), 20-(hydroxymethyl)pregna-4-en-3-one and/or 20-(hydroxymethyl)pregna-1,4-dien-3-one, comprising
(a) (i) making a cell that underexpresses (as compared to a wild type or unmanipulated cell) or does not express any one, or several of, or all KsdA-, CxgA-, CxgB-, CxgC- and/or CxgD polypeptides in the cell; and, (ii) culturing the cell under conditions wherein androstenedione is produced,
wherein underexpressing or inhibiting the activity of the KsdA-, CxgA-, CxgB-, CxgC- and/or CxgD polypeptides in the cell results production of an androstenedione (AD) of relative purity, or substantially free of androstadienedione (ADD), 20-(hydroxymethyl) pregna-4-en-3-one and/or 20-(hydroxymethyl)pregna-1,4-dien-3-one;
(b) the process of (a), wherein the underexpression of or inhibition of activity of the KsdA-, CxgA-, CxgB-, CxgC- and/or CxgD polypeptides in the cell is by practicing the method of claim 38;
(c) the process of (a) or (b), wherein the cell underexpresses a KsdA-, CxgA-, CxgB-, CxgC- and/or CxgD polypeptide (as compared to a wild type or unmanipulated cell) by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;
(d) the process of (a) or (b), wherein the cell underproduces an androstenedione (AD) of relative purity, or substantially free of androstadienedione (ADD), 20-(hydroxymethyl) pregna-4-en-3-one and/or 20-(hydroxymethyl)pregna-1,4-dien-3-one by at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0% or 90.0% or more;
(e) the process of any of (a) to (d), wherein the cell produces at least about 1.0%, 2.0%, 3.0%, 4.0%, 5.0%, 10.0%, 15%, 20.0%, 25.0%, 30.0%, 35.0%, 40.0%, 45.0%, 50.0%, 55.0%, 60.0%, 65.0%, 70.0%, 75.0%, 80.0%, 85.0%, 90.0% or 95.0% or more % fewer (lesser amounts of) impurities in the AD synthesis process; or
(f) the process of (e), wherein the fewer impurities comprise fewer (lesser amounts of) androstadienedione (ADD), 20-(hydroxymethyl)pregna-4-en-3-one and/or 20-(hydroxymethyl)pregna-1,4-dien-3-one.

41. A kit comprising (a) the nucleic acid of claim 1; the probe of claim 2; the vector, expression cassette or cloning vehicle of claim 3; or, the host cell or a transformed cell of claim 4; or (b) the kit of (a), further comprising instructions for practicing any one of the methods of claim 17 to claim 24, or claim 27 to claim 40.

42. A kit comprising (a) a polypeptide of claim 9; an antibody of claim 14; a hybridoma of claim 15, an array of claim 16, a heterodimer of claim 11; or (b) the kit of (a), further comprising instructions for practicing any one of the methods of claim 17 to claim 24, or claim 27 to claim 40.
